(19) United States
(12) Patent Application Publication
(10) Pub. No.: US 2007/0249599 A1
(43) Pub. Date: Oct. 25, 2007

(54) Novel Chemical Compounds

(76) Inventors: Kevin J. Duffy, Collegeville, PA (US); Duke M. Fitch, Collegeville, PA (US); Steven Neal Goodman, King of Prussia, PA (US); Masaichi Hasegawa, Tsukuba-shi (JP); Neil W. Johnson, Collegeville, PA (US); Jiri Kasparec, Collegeville, PA (US); Antony N. Shaw, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/590,623

(22) PCT Filed: Feb. 24, 2005
(86) PCT No.: PCT/US05/06022

§ 371 Date: Aug. 24, 2006

Related U.S. Application Data
(60) Provisional application No. 60/547,543, filed on Feb. 25, 2004.

(51) Int. Cl. A61K 31/427; A61K 31/4439; A61K 31/454; A61K 31/496; A61K 31/5355; C07D 277/04; C07D 417/10

(52) U.S. Cl. 514/234.5; 514/254.02; 514/326; 514/342; 514/369; 544/133; 544/369; 546/209; 546/270.7; 548/184

(57) ABSTRACT

This invention relates to the newly identified compounds for inhibiting hYAK3 proteins and methods for treating diseases associated with the imbalance or inappropriate activity of hYAK3 proteins.

### FIELD OF THE INVENTION

This invention relates to newly identified compounds for inhibiting hYAK3 proteins and methods for treating diseases associated with hYAK3 activity.

### BACKGROUND OF THE INVENTION

A number of polypeptide growth factors and hormones mediate their cellular effects through a signal transduction pathway. Transduction of signals from the cell surface receptors for these ligands to intracellular effectors frequently involves phosphorylation or dephosphorylation of specific protein substrates by regulatory protein serine/threonine kinases (PSTK) and phosphatases. Serine/threonine phosphorylation is a major mediator of signal transduction in multicellular organisms. Receptor-bound, membrane-bound and intracellular PSTKs regulate cell proliferation, cell differentiation and signalling processes in many cell types.

Aberrant protein serine/threonine kinase activity has been implicated or is suspected in a number of pathologies such as rheumatoid arthritis, psoriasis, septic shock, bone loss, many cancers and other proliferative diseases. Accordingly, serine/threonine kinases and the signal transduction pathways which they are part of are potential targets for drug design.

A subset of PSTKs are involved in regulation of cell cycling. These are the cyclin-dependent kinases or CDKs (Peter and Herskowitz, Cell 1994: 79, 181-184). CDKs are activated by binding to regulatory proteins called cyclins and control passage of the cell through specific cell cycle checkpoints. For example, CDK2 complexed with cyclin E allows cells to progress through the G1 to S phase transition. The complexes of CDKs and cyclins are subject to inhibition by low molecular weight proteins such as p16 (Serrano et al, Nature 1993: 366, 704), which binds to and inhibits CDK4. Deletions or mutations in p16 have been implicated in a variety of tumors (Kamb et al, Science 1994: 264, 436-440). Therefore, the proliferative state of cells and diseases associated with this state are dependent on the activity of CDKs and their associated regulatory molecules. In diseases such as cancer where inhibition of proliferation is desired, compounds that inhibit CDKs may be useful therapeutic agents. Conversely, activators of CDKs may be useful where enhancement of proliferation is needed, such as in the treatment of immunodeficiency.

YAK1, a PSTK with sequence homology to CDKs, was originally identified in yeast as a mediator of cell cycle arrest caused by inactivation of the cAMP-dependent protein kinase PKA (Garrett et al, Mol Cell Biol. 1991: 11-6045-4052). YAK1 kinase activity is low in cycling yeast but increases dramatically when the cells are arrested prior to the S-G2 transition. Increased expression of YAK1 causes growth arrest in yeast cells deficient in PKA. Therefore, YAK1 can act as a cell cycle suppressor in yeast.

Our U.S. Pat. No. 6,323,318 describes two novel human homologs of yeast YAK1 termed hYAK3-2, one protein longer than the other by 20 amino acids. hYAK3-2 proteins (otherwise reported as REDK-L and REDK-S in Blood, 1 May 2000, Vol 95, No. 9, pp 2838) are primarily localized in the nucleus. hYAK-2 proteins (hereinafter simply referred as hYAK3 or hYAK3 proteins) are present in hematopoietic tissues, such as bone marrow and fetal liver, but the RNA is expressed at significant levels only in erythroid or erthropoietin (EPO)-responsive cells. Two forms of REDK cDNAs appear to be alternative splice products. Antisense REDK oligonucleotides promote erythroid colony formation by human bone marrow cells, without affecting colony-forming unit (CFU)-GM, CFU-C, or CFU-GEMM numbers. Maximal numbers of CFU-E and burst-forming unit-erythroid were increased, and CFU-E displayed increased sensitivity to suboptimal EPO concentrations. The data indicate that REDK acts as a brake to retard erythropoiesis. Thus inhibitors of hYAK3 proteins are expected to stimulate proliferation of cells in which it is expressed. More particularly, inhibitors of hYAK3 proteins are useful to treat or prevent diseases of the erythroid and hematopoietic systems mediated the imbalance or inappropriate activity of hYAK3 proteins, including but not limited to, anemia, anemias due to renal insufficiency or to chronic disease, such as autoimmunity, HIV, or cancer, and drug-induced anemias, myelodysplastic syndrome, aplastic anemia and myelosuppression, and cytopenia.

### SUMMARY OF THE INVENTION

In the first aspect, the present invention relates to a compound of the formula I, and/or a pharmaceutically acceptable salt, hydrate, solvate, or pro-drug thereof, 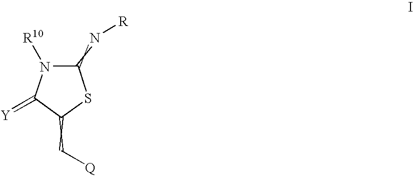
wherein:
R is selected from: hydrogen, aryl, substituted aryl, cycloalkyl, substituted cycloalkyl, C₁₋₆alkyl and substituted C₁₋₆alkyl;
R¹⁰ is selected from: hydrogen, C₁₋₆alkyl, —(CH₂)ₘOH and —(CH₂)ₘCOOH,
   where m is 0 to 6;
Y is selected from: ═O, ═S and ═NR¹¹,
   where R¹¹ is selected from: hydrogen, C₁₋₆alkyl, —(CH₂)ₚOH and —(CH₂)ₚCOOH,
   where p is 0 to 6; and
Q is a radical or substituted radical of the formula, 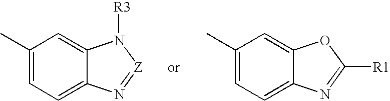
   in which Z is N or C—R²;
   wherein R² is hydrogen, —NH₂, —C₁₋₆alkyl, substituted —C₁₋₆alkyl, —CF₃, aryl or a radical or substituted radical of the formula 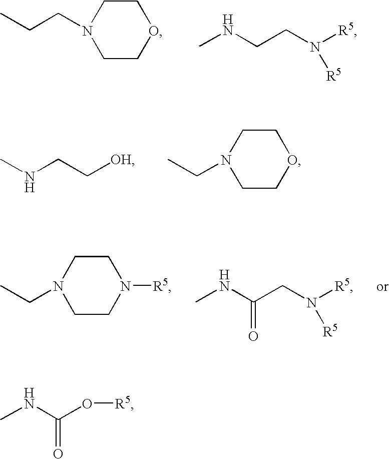
wherein R⁵ is selected from: hydrogen, -C₁₋₆alkyl and substituted -C_{1 6}alkyl; and
R³ is hydrogen, -C₁₋₆alkyl, substituted -C₁₋₆alkyl or C₃₋₁₂cycloalkyl; and
R¹ is hydrogen, -C₁₋₆alkyl, substituted -C₁₋₆alkyl, amino, mono substituted amino, disubstituted amino and trifluoromethyl.

In a second aspect, the instant invention relates a method of inhibiting hYAK3 in a mammal; comprising, administering to the mammal a therapeutically effective amount of a compound of the Formula I, or a pharmaceutically acceptable salt, hydrate, solvate or pro-drug thereof.

In a third aspect of the present invention, there is provided a pharmaceutical composition including a therapeutically effective amount of a compound of Formula I, or a pharmaceutically acceptable salt, hydrate, solvate or pro-drug thereof and one or more of pharmaceutically acceptable carriers, diluents and excipients.

In a fourth aspect of the present invention, there is provided the use of a compound of Formula I, or a pharmaceutically acceptable salt, hydrate, solvate or pro-drug thereof in the preparation of a medicament for use in the treatment or prevention of a disorder of the erythroid and hematopoietic systems mediated by the imbalance or inappropriate activity of hYAK3 proteins, including but not limited to, anemia, anemias due to renal insufficiency or to chronic disease, such as autoimmunity, HIV, or cancer, and drug-induced anemias, myelodysplastic syndrome, aplastic anemia and myelosuppression, and cytopenia.

In a fifth aspect, the present invention relates to a method of treating or preventing diseases of the erythroid and hematopoietic systems, caused by the hYAK3 imbalance or inappropriate activity including, but not limited to, anemia, anemias due to renal insufficiency or to chronic disease, such as autoimmunity, HIV, or cancer, and drug-induced anemias, myelodysplastic syndrome, aplastic anemia and myelosuppression, and cytopenia; comprising, administering to a mammal a therapeutically effective amount of a compound of Formula I, or a pharmaceutically acceptable salt, hydrate, solvate or pro-drug thereof and one or more of pharmaceutically acceptable carriers, diluents and excipients.

In a six aspect, the present invention relates to a method of treating or preventing anemia, anemias due to renal insufficiency or to chronic disease, such as autoimmunity, HIV, or cancer, and drug-induced anemias, myelodysplastic syndrome, aplastic anemia and myelosuppression, and cytopenia; comprising, administering to a mammal a therapeutically effective amount of a compound of Formula I, or a pharmaceutically acceptable salt, hydrate, solvate or pro-drug thereof and one or more of pharmaceutically acceptable carriers, diluents and excipients.

Also included in the present invention are methods of co-administering the presently invented hYAK3 inhibiting compounds with further active ingredients.

### DETAILED DESCRIPTION

This invention relates to compounds of Formula I and/or pharmaceutically acceptable salts, hydrates, solvates, and pro-drugs thereof.

Included in the presently invented compounds of Formula I are those having Formula II, and/or pharmaceutically acceptable salts, hydrates, solvates, or pro-drugs thereof, 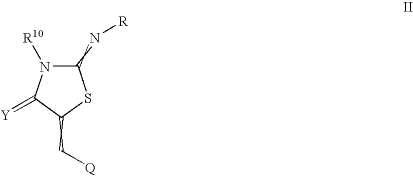
wherein:
R is selected from: hydrogen, C₁-C₁₂aryl, substituted C₁-C₁₂aryl, cycloalkyl, substituted cycloalkyl, C₁₋₆alkyl and substituted C₁₋₆alkyl;
R¹⁰ is selected from: hydrogen, C₁₋₆alkyl, —(CH₂)ₘOH and —(CH₂)ₘCOOH,
   where m is 0 to 6;
Y is selected from: ═O, ═S and ═NR¹¹,
   where R¹¹ is selected from: hydrogen, C₁₋₆alkyl, —(CH₂)ₚOH and —(CH₂)ₚCOOH,
   where p is 0 to 6; and
Q is a radical of the formula, 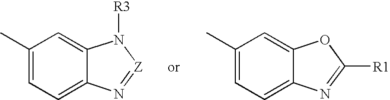
   in which Z is N or C—R²;
   wherein R² is hydrogen, —NH₂, -C₁₋₆alkyl, substituted -C₁₋₆alkyl, —CF₃, aryl or a radical of the formula 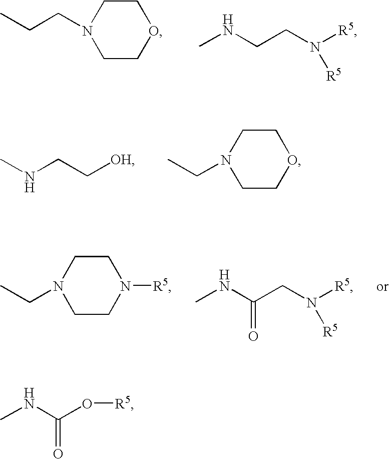
wherein R⁵ is selected from: hydrogen, -C₁₋₆alkyl and substituted -C₁₋₆alkyl; and
R³ is hydrogen, -C₁₋₆alkyl, substituted -C₁₋₆alkyl or C₃₋₁₂cycloalkyl; and
R¹ is hydrogen, -C₁₋₆alkyl, substituted -C₁₋₆alkyl, amino, mono substituted amino, disubstituted amino and trifluoromethyl.

Included in the presently invented compounds of Formula I are those having Formula III, and/or pharmaceutically acceptable salts, hydrates, solvates, or pro-drugs thereof, 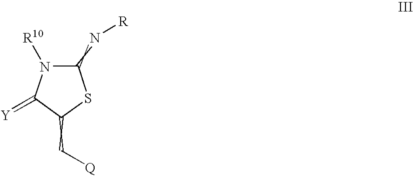
wherein:
R is selected from: hydrogen, C₁-C₁₂aryl, substituted C₁-C₁₂aryl, cycloalkyl, substituted cycloalkyl, C₁₋₆alkyl, —(CH₂)ₙ—NR^{k}R^{h}, —C(═NH)NH₂, —(CH₂)₂N(CH₃)₂, —C(═O)CH₃, —(CH₂)₂OCH₃, —(CH₂)₂OH, —(CH₂)₂C(CH₃)₃ and —(CH₂)CH(CH₃)₂, —C(═O)Ph, —C(═O)CH₂NHBOC, —(CH₂)₂CH(CH₃)₂,
   where n is 0 to 6, and
   R^{k} and R^{h} are independently selected form hydrogen, C₁₋₆alkyl and substituted C₁₋₆alkyl;
R¹⁰ is selected from: hydrogen, C₁₋₆alkyl, —(CH₂)ₘOH and —(CH₂)ₘCOOH,
   where m is 0 to 6;
Y is selected from: ═O, ═S and ═NR¹¹,
   where R¹¹ is selected from: hydrogen, C₁₋₆alkyl, —(CH₂)ₚOH and —(CH₂)ₚCOOH,
   where p is 0 to 6; and
Q is a radical of the formula, 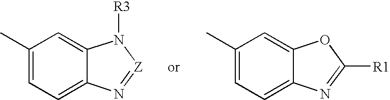
in which Z is N or C—R²;
wherein R² is hydrogen, —NH₂, -C₁₋₆alkyl, substituted -C₁₋₆alkyl, —CF₃, aryl or a radical of the formula 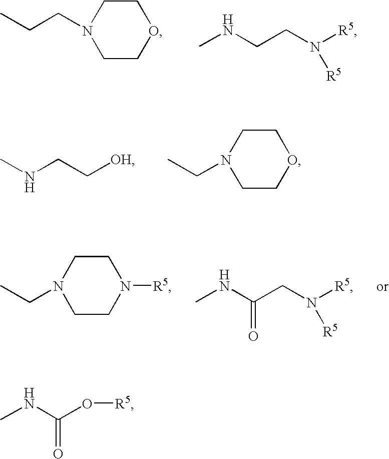
wherein R⁵ is selected from: hydrogen, -C₁₋₆alkyl and substituted -C₁₋₆alkyl; and
R³ is hydrogen, -C₁₋₆alkyl, substituted -C₁₋₆alkyl or C₃₋₁₂cycloalkyl; and
R¹ is hydrogen, -C₁₋₆alkyl, substituted -C₁₋₆alkyl, amino, mono substituted amino, disubstituted amino and trifluoromethyl.

Included in the presently invented compounds of Formula I are those having Formula IV, and/or pharmaceutically acceptable salts, hydrates, solvates, or pro-drugs thereof, 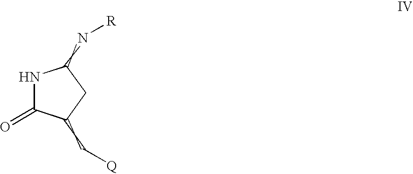
in which
R is 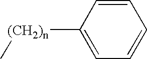
in which the phenyl radical is optionally and independently substituted with up to three substituents selected form: halogen, -C₁₋₆alkyl, —OC₁₋₆alkyl, —CF₃, —CN, —CO₂H, —SO₂NH₂, —CONH₂; or
   R is a radical of the formula 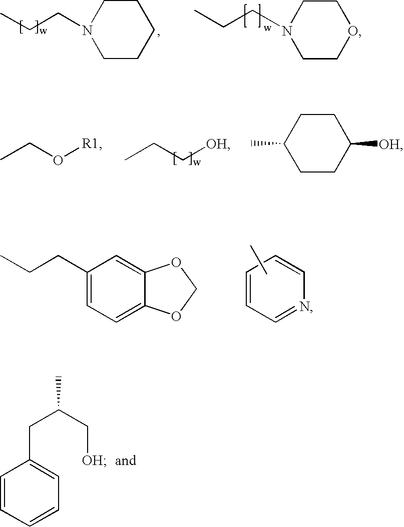
   Q is a radical of the formula 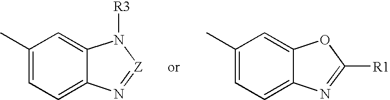
   in which Z is N or C—R2;

wherein R2 is hydrogen, —NH₂, -C₁₋₆alkyl, —CF₃, or a radical of the formula 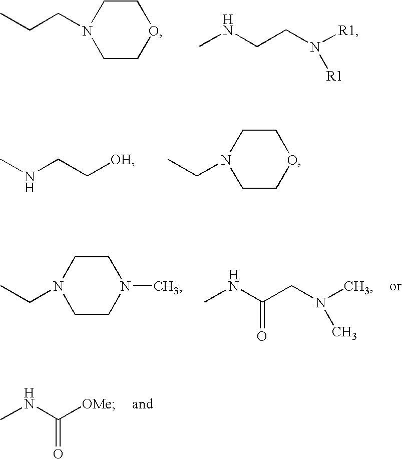
R3 is -C₁₋₆ alkyl, or a radical of the formula 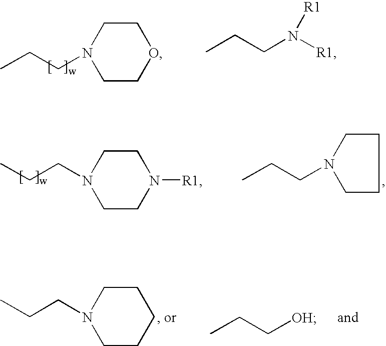
n equals zero to two;
w equals one to two; and
R1 is -C₁₋₆alkyl.

Included in the presently invented compounds of Formula IV are those in which R is phenyl optionally and independently substituted with up to three substituents selected form: halogen, -C₁₋₆alkyl, —OC₁₋₆alkyl, —CF₃, —CN, —CO₂H, —SO₂NH₂, —CONH₂.

Included in the presently invented compounds of Formula IV are those in which R is defined as a radical of the formula 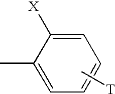
in which X is halogen or CF3; and T is selected from: hydrogen, halogen, -C₁₋₆alkyl, —OC₁₋₆alkyl, —CF₃, —CN, —CO₂H, —SO₂NH₂, —CONH₂.

Included in the presently invented compounds of Formula IV are those in which R is defined as a radical of the formula 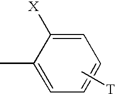
in which X is halogen or —CF3; and T is selected from: hydrogen, halogen, -C₁₋₆alkyl, —OC₁₋₆alkyl, —CF₃, —CN, —CO₂H, —SO₂NH₂, —CONH₂; and Q is 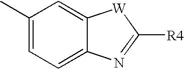
in which R4 is methyl or hydrogen, and W is O or N—R1, in which R1 is -C₁₋₆alkyl.

Included among the presently invented compounds are:

(5Z)-2-[(2-Chlorophenyl)amino]-5-[(1-methyl-1H-benzimidazol-6-yl)methylidene]-1,3-thiazol-4(5H)-one;

(5Z)-2-[(2,6-dichlorophenyl)amino]-5-[(1-methyl-1H-benzimidazol-6-yl)methylidene]-1,3-thiazol-4(5H)-one;

(5Z)-2-[(2,6-difluorophenyl)amino]-5-[(1-methyl-1H-benzimidazol-6-yl)methylidene]-1,3-thiazol-4(5H)-one;

(5Z)-2-[(2,4-dimethylphenyl)amino]-5-[(1-methyl-1H-benzimidazol-6-yl)methylidene]-1,3-thiazol-4(5H)-one;

(5Z)-5-[(1-methyl-1H-benzimidazol-6-yl)methylidene]-2-{[2-(methyloxy)phenyl]amino}-1,3-thiazol-4(5H)-one;

(5Z)-5-[(1-methyl-1H-benzimidazol-6-yl)methylidene]-2-{[2-(trifluoromethyl)phenyl]amino}-1,3-thiazol-4(5H)-one;

(5Z)-2-[(2,4-difluorophenyl)amino]-5-[(1-methyl-1H-benzimidazol-6-yl)methylidene]-1,3-thiazol-4(5H)-one;

(5Z)-2-[(2-chloro-4-fluorophenyl)amino]-5-[(1-methyl-1H-benzimidazol-6-yl)methylidene]-1,3-thiazol-4(5H)-one;

(5Z)-2-[(2-Chlorophenyl)-amino]-5-[(1,2-dimethyl-1H-benzimidazol-6-yl)-methylidene]-1,3-thiazol-4(5H)-one;

(5Z)-2-[(2,6-dichlorophenyl)amino]-5-[(1,2-dimethyl-1H-benzimidazol-6-yl)methylidene]-1,3-thiazol-4(5H)-one;

(5Z)-2-[(2,6-difluorophenyl)amino]-5-[(1,2-dimethyl-1H-benzimidazol-6-yl)methylidene]-1,3-thiazol-4(5H)-one;

(5Z)-5-[(1,2-dimethyl-1H-benzimidazol-6-yl)methylidene]-2-[(2,4-dimethylphenyl)amino]-1,3-thiazol-4(5H)-one;

(5Z)-2-[(2,4-difluorophenyl)amino]-5-[(1,2-dimethyl-1H-benzimidazol-6-yl)methylidene]-1,3-thiazol-4(5H)-one;

(5Z)-2-[(2-chloro-4-fluorophenyl)amino]-5-[(1,2-dimethyl-1H-benzimidazol-6-yl)methylidene]-1,3-thiazol-4(5H) -one;

(5Z)-2-[(2-Chlorophenyl)-amino]-5-({1-[2-(4-morpholinyl)ethyl]-1H-benzimidazol-6-yl}-methylidene)-1,3-thiazol-4(5H)-one;

(5Z)-2-[(2,6-dichlorophenyl)amino]-5-({1-[2-(4-morpholinyl)ethyl]-1H-benzimidazol-6-yl}methylidene)-1,3-thiazol-4(5H)-one;

(5Z)-2-[(2-chloro-4-fluorophenyl)amino]-5-({1-[2-(4-morpholinyl)ethyl]-1H-benzimidazol-6-yl}methylidene)-1,3-thiazol-4(5H)-one;

(5Z)-2-[(2-chlorophenyl)amino]-5-({1-[2-(dimethylamino)ethyl]-1H-benzimidazol-6-yl}methylidene)-1,3-thiazol-4(5H)-one;

(5Z)-2-[(2,6-dichlorophenyl)amino]-5-({1-[2-(dimethylamino)ethyl]-1H-benzimidazol-6-yl}methylidene)-1,3-thiazol-4(5H)-one;

(5Z)-2-[(2,4-difluorophenyl)amino]-5-({1-[2-(dimethylamino)ethyl]-1H-benzimidazol-6-yl}methylidene)-1,3-thiazol-4(5H)-one;

(5Z)-5-({1-[2-(dimethylamino)ethyl]-1H-benzimidazol-6-yl}methylidene)-2-(phenylamino)-1,3-thiazol-4(5H)-one;

(5Z)-2-[(2-chlorophenyl)amino]-5-({1-[2-(diethylamino)ethyl]-1H-benzimidazol-6-yl}methylidene)-1,3-thiazol-4(5H)-one;

(5Z)-2-[(2,6-dichlorophenyl)amino]-5-({1-[2-(diethylamino)ethyl]-1H-benzimidazol-6-yl}methylidene)-1,3-thiazol-4(5H)-one;

(5Z)-2-[(2-chlorophenyl)amino]-5-({1-[3-(4-morpholinyl)propyl]-1H-benzimidazol-6-}methylidene)-1,3-thiazol-4(5H)-one;

(5Z)-2-[(2,6-dichlorophenyl)amino]-5-({1-[3-(4-morpholinyl)propyl]-1H-benzimidazol-6-yl}methylidene)-1,3-thiazol-4(5H)-one;

(5Z)-2-[(2-chlorophenyl)amino]-5-({1-[3-(4-methyl-1-piperazinyl)propyl]-1H-benzimidazol-6-yl}methylidene)-1,3-thiazol-4(5H)-one;

(5Z)-2-[(2,6-dichlorophenyl)amino]-5-({1-[3-(4-methyl-1-piperazinyl)propyl]-1H-benzimidazol-6-yl}methylidene)-1,3-thiazol-4(5H)-one;

(5Z)-2-[(2-chlorophenyl)amino]-5-({1-[2-(1-pyrrolidinyl)ethyl]-1H-benzimidazol-6-yl}methylidene)-1,3-thiazol-4(5H)-one;

(5Z)-2-[(2,6-dichlorophenyl)amino]-5-({1-[2-(1-pyrrolidinyl)ethyl]-1H-benzimidazol-6-yl}methylidene)-1,3-thiazol-4(5H)-one;

(5Z)-2-[(2-chlorophenyl)amino]-5-({1-[2-(1-piperidinyl)ethyl]-1H-benzimidazol-6-yl}methylidene)-1,3-thiazol-4(5H) -one;

(5Z)-2-[(2,6-dichlorophenyl)amino]-5-({1-[2-(1-piperidinyl)ethyl]-1H-benzimidazol-6-yl}methylidene)-1,3-thiazol-4(5H) -one;

(5Z)-2-[(2,6-difluorophenyl)amino]-5-({1-[2-(1-piperidinyl)ethyl]-1H-benzimidazol-6-yl}methylidene)-1,3-thiazol-4(5H)-one;

(5Z)-2-[(2-Chlorophenyl)amino]-5-({1-[2-(dimethylamino)ethyl]-2-methyl-1H-benzimidazol-6-yl}methylidene)-1,3-thiazol-4(5H)-one;

(5Z)-2-[(2,6-dichlorophenyl)amino]-5-({1-[2-(dimethylamino)ethyl]-2-methyl-1H-benzimidazol-6-yl}methylidene)-1,3-thiazol-4(5H)-one;

(5Z)-2-[(2,4-difluorophenyl)amino]-5-({1-[2-(dimethylamino)ethyl]-2-methyl-1H-benzimidazol-6-yl}methylidene)-1,3-thiazol-4(5H)-one;

(5Z)-5-({1-[2-(dimethylamino)ethyl]-2-methyl-1H-benzimidazol-6-yl}methylidene)-2-(phenylamino)-1,3-thiazol-4(5H)-one;

(5Z)-2-[(2,6-dichlorophenyl)amino]-5-{[1-(2-hydroxyethyl)-2-methyl-1H-benzimidazol-6-yl]methylidene}-1,3-thiazol-4(5H)-one;

(5Z)-2-[(2-chlorophenyl)amino]-5-({2-methyl-1-[2-(1-piperidinyl)ethyl]-1H-benzimidazol-6-yl}methylidene)-1,3-thiazol-4(5H)-one;

(5Z)-2-[(2,6-dichlorophenyl)amino]-5-({2-methyl-1-[2-(1-piperidinyl)ethyl]-1H-benzimidazol-6-yl}methylidene)-1,3-thiazol-4(5H)-one;

(5Z)-2-[(2,6-difluorophenyl)amino]-5-({2-methyl-1-[2-(1-piperidinyl)ethyl]-1H-benzimidazol-6-yl}methylidene)-1,3-thiazol-4(5H)-one;

(5Z)-2-[(2-chlorophenyl)amino]-5-[(1-methyl-2-{[2-(4-morpholinyl)ethyl]amino}-1H-benzimidazol-6-yl)methylidene]-1,3-thiazol-4(5H)-one;

(5Z)-2-[(2,6-dichlorophenyl)amino]-5-[(1-methyl-2-{[2-(4-morpholinyl)ethyl]amino}-1H-benzimidazol-6-yl)methylidene]-1,3-thiazol-4(5H)-one;

(5Z)-2-[(2,4-difluorophenyl)amino]-5-[(1-methyl-2-{[2-(4-morpholinyl)ethyl]amino}-1H-benzimidazol-6-yl)methylidene]-1,3-thiazol-4(5H)-one;

(5Z)-2-[(2-chlorophenyl)amino]-5-[(2-{[2-(dimethylamino)ethyl]amino}-1-methyl-1H-benzimidazol-6-yl)methylidene]-1,3-thiazol-4(5H)-one;

(5Z)-2-[(2-chlorophenyl)amino]-5-({2-[(2-hydroxyethyl)amino]-1-methyl-1H-benzimidazol-6-yl}methylidene)-1,3-thiazol-4(5H)-one;

(5Z)-2-[(2-chlorophenyl)amino]-5-{[1-methyl-2-(4-morpholinylmethyl)-1H-benzimidazol-6-yl]methylidene}-1,3-thiazol-4(5H)-one;

(5Z)-2-[(2,6-dichlorophenyl)amino]-5-{[1-methyl-2-(4-morpholinylmethyl)-1H-benzimidazol-6-yl}methylidene}-1,3-thiazol-4(5H)-one;

(5Z)-2-[(2-chlorophenyl)amino]-5-({1-methyl-2-[(4-methyl-1-piperazinyl)methyl]-1H-benzimidazol-6-yl}methylidene)-1,3-thiazol-4(5H)-one;

(5Z)-2-[(2,6-dichlorophenyl)amino]-5-({1-methyl-2-[(4-methyl-1-piperazinyl)methyl]-1H-benzimidazol-6-yl}methylidene)-1,3-thiazol-4(5H)-one;

(5Z)-2-[(2-Chlorophenyl)-amino]-5-{[1-methyl-2-(trifluoromethyl)-1H-benzimidazol-6-yl]-methylidene}-1,3-thiazol-4(51H)-one;

(5Z)-2-[(2,6-dichlorophenyl)amino]-5-{[1-methyl-2-(trifluoromethyl)-1H-benzimidazol-6-yl]methylidene}-1,3-thiazol-4(5H)-one;

(5Z)-2-[(2,6-dichlorophenyl)amino]-5-{[1-[2-(dimethylamino)ethyl]-2-(trifluoromethyl)-1H-benzimidazol-6-yl]methylidene}-1,3-thiazol-4(5H)-one;

(5Z)-2-[(2-chlorophenyl)amino]-5-{[2-(1,1-dimethylethyl)-1-methyl-1H-benzimidazol-6-yl]methylidene}-1,3-thiazol-4(5H)-one;

(5Z)-2-[(2,6-dichlorophenyl)amino]-5-{[2-(1,1-dimethylethyl)-1-methyl-1H-benzimidazol-6-yl]methylidene}-1,3-thiazol-4(5H)-one;

(5Z)-2-[(2-Chlorophenyl)amino]-5-[(1-methyl-1H-1,2,3-benzotriazol-6-yl)methylidene]-1,3-thiazol-4(5H)-one;

(5Z)-2-[(2,6-dichlorophenyl)amino]-5-[(1-methyl-1H-1,2,3-benzotriazol-6-yl)methylidene]-1,3-thiazol-4(5H) -one;

(5Z)-2-[(2-chlorophenyl)amino]-5-({1-[2-(dimethylamino)ethyl]-1H-1,2,3-benzotriazol-6-yl}methylidene)-1,3-thiazol-4(5H)-one;

(5Z)-2-[(2,6-dichlorophenyl)amino]-5-({1-[2-(dimethylamino)ethyl]-1H-1,2,3-benzotriazol-6-yl}methylidene)-1,3-thiazol-4(5H)-one;

2-(2,6-Dichloro-phenylimino)-5-(2-methyl-benzooxazol-6-yl-methylene)-thiazolidin-4-one;

2-(2,6-Difluoro-phenylimino)-5-(2-methyl-benzooxazol-6-yl-methylene)-thiazolidin-4-one;

2-(2-Fluoro-phenylimino)-5-(2-methyl-benzooxazol-6-yl-methylene)-thiazolidin-4-one;

2-(2-Chloro-phenylimino)-5-(2-methyl-benzooxazol-6-ylmethylene)-thiazolidin-4-one;

2-(2-Trifluromethyl-phenylimino)-5-(2-methyl-benzooxazol-6-ylmethylene)-thiazolidin-4-one;

2-(2,4-Difluoro-phenylimino)-5-(2-methyl-benzooxazol-6-ylmethylene)-thiazolidin-4-one;

2-(2,5-Dichloro-phenylimino)-5-(2-methyl-benzooxazol-6-ylmethylene)-thiazolidin-4-one;

2-(2,4-Dimethyl-phenylimino)-5-(2-methyl-benzooxazol-6-ylmethylene)-thiazolidin-4-one;

2-(4-Cyano-phenylimino)-5-(2-methyl-benzooxazol-6-ylmethylene)-thiazolidin-4-one;

4-[5-(2-Methyl-benzooxazol-6-ylmethylene)-4-oxo-thiazolidin-2-ylideneamino]-benzoic acid;

2-(2,4-Dichloro-phenylimino)-5-(2-methyl-benzooxazol-6-ylmethylene)-thiazolidin-4-one;

2-(2,5-Difluoro-phenylimino)-5-(2-methyl-benzooxazol-6-ylmethylene)-thiazolidin-4-one;

5-(2-Methyl-benzooxazol-6-ylmethylene)-2-phenylimino-thiazolidin-4-one;

5-(2-Methyl-benzooxazol-6-ylmethylene)-2-(2-piperidin-1-yl-ethylimino)-thiazolidin-4-one;

2-(2-Methoxy-ethylimino)-5-(2-methyl-benzooxazol-6-ylmethylene)-thiazolidin-4-one;

5-(2-Methyl-benzooxazol-6-ylmethylene)-2-(3-morpholin-4-yl-propylimino)-thiazolidin-4-one;

3-[5-(2-Methyl-benzooxazol-6-ylmethylene)-4-oxo-thiazolidin-2-ylideneamino]-benzenesulfonamide;

2-(4-Hydroxy-butylimino)-5-(2-methyl-benzooxazol-6-ylmethylene)-thiazolidin-4-one;

2-(trans-4-Hydroxy-cyclohexylimino)-5-(2-methyl-benzooxazol-6-ylmethylene)-thiazolidin-4-one;

5-(2-Methyl-benzooxazol-6-ylmethylene)-2-phenethylimino-thiazolidin-4-one;

4-{2-[5-(2-Methyl-benzooxazol-6-ylmethylene)-4-oxo-thiazolidin-2-ylideneamino]-ethyl}-benzenesulfonamide;

2-(2-Benzo[1,3]dioxol-5-yl-ethylimino)-5-(2-methyl-benzooxazol-6-ylmethylene)-thiazolidin-4-one;

2-(4-Chloro-phenylimino)-5-(2-methyl-benzooxazol-6-ylmethylene)-thiazolidin-4-one;

5-(2-Methyl-benzooxazol-6-ylmethylene)-2-(pyridin-3-ylimino)-thiazolidin-4-one;

3-[5-(2-Methyl-benzooxazol-6-ylmethylene)-4-oxo-thiazolidin-2-ylideneamino]-benzamide;

2-(2-Hydroxy-ethylimino)-5-(2-methyl-benzooxazol-6-ylmethylene)-thiazolidin-4-one;

2-(1-Hydroxymethyl-2-phenyl-ethylimino)-5-(2-methyl-benzooxazol-6-ylmethylene)-thiazolidin-4-one;

N-{6-[2-(2-Bromo-phenylimino)-4-oxo-thiazolidin-5-ylidenemethyl]-1H-benzoimidazol-2-yl}-2-dimethylamino-acetamide;

Methyl (5-{(Z)-[2-[(2-bromophenyl)amino]-4-oxo-1,3-thiazol-5(4H)-ylidene]methyl}-1H-benzimidazol-2-yl)carbamate;

(5Z)-2-[(2-chlorophenyl)amino]-5-{[1-(3,3-dimethylbutyl)-1H-benzimidazol-6-yl]methylidene}-1,3-thiazol-4(5H)-one;

(5Z)-2-[(2,6-dichlorophenyl)amino]-5-{[1-(3,3-dimethylbutyl)-1H-benzimidazol-6-yl]methylidene}-1,3-thiazol-4(5H)-one, trifluoroacetate salt;

(5Z)-5-{[1-(2-cyclopropylethyl)-1H-benzimidazol-6-yl]methylidene}-2-[(2,6-difluorophenyl)amino]-1,3-thiazol-4(5H)-one;

(5Z)-5-{[1-(2-cyclohexylethyl)-1H-benzimidazol-6-yl]methylidene}-2-[(2,6-dichlorophenyl)amino]-1,3-thiazol-4(5H)-one, trifluoroacetate salt;

(2Z,5Z)-2-[(2,6-dichlorophenyl)imino]-5-[(2-phenyl-1H-benzimidazol-6-yl)methylidene]-1,3-thiazolidin-4-one, piperidine salt;

(5Z)-2-[(2-chlorophenyl)amino]-5-{[1-(2-cyclopropylethyl)-1H-benzimidazol-6-yl]methylidene}-1,3-thiazol-4(5H)-one;

(5Z)-5-{[1-(2-cyclopropylethyl)-1H-benzimidazol-6-yl]methylidene}-2-[(2,6-dichlorophenyl)amino]-1,3-thiazol-4(5H)-one;

(2Z,5Z)-2-[(2,6-dichlorophenyl)imino]-5-{[2-(1-methylethyl)-1H-benzimidazol-6-yl]methylidene}-1,3-thiazolidin-4-one;

(2Z,5Z)-2-[(2,6-dichlorophenyl)imino]-5-{[2-(2-methylpropyl)-1H-benzimidazol-6-yl]methylidene}-1,3-thiazolidin-4-one, piperidine salt;

(2Z, 5Z)-2-[(2,6-dichlorophenyl)imino]-5-{[2-(3-pyridinyl)-1H-benzimidazol-6-yl]methylidene}-1,3-thiazolidin-4 one;

(2Z,5Z)-2-[(2,6-dichlorophenyl)imino]-5-{[2-(hydroxynmethyl)-1H-benzimidazol-5-yl]methylidene}-1,3-thiazolidin-4-one;

(2Z,5Z)-2-[(2,6-dichlorophenyl)imino]-5-{[2-(2-hydroxyethyl)-1H-benzimidazol-5-yl]methylidene}-1,3-thiazolidin-4-one;

(2Z,5Z)-2-[(2,6-dichlorophenyl)imino]-5-{[2-(2-pyridinyl)-1H-benzimidazol-6-yl]methylidene}-1,3-thiazolidin-4-one;

(5Z)-5-{[1-(2-cyclopentylethyl)-1H-benzimidazol-6-yl]methylidene}-2-[(2,6-dichlorophenyl)amino]-1,3-thiazol-4(5H)-one;

(5Z)-2-[(2-chlorophenyl)amino]-5-{[1-(2-cyclopentylethyl)-1H-benzimidazol-6-yl]methylidene}-1,3-thiazol-4(5H)-one;

(2Z,5Z)-2-[(2,6-dichlorophenyl)imino]-5-[(2-methyl-1H-benzimidazol-6-yl)methylidene]-1,3-thiazolidin-4-one;

(2Z,5Z)-2-[(2,6-dichlorophenyl)imino]-5-{[2-(4-pyridinyl)-1H-benzimidazol-5-yl]methylidene}-1,3-thiazolidin-4-one;

(2Z,5Z)-5-{[1-(2-cyclopropylethyl)-2-(3-pyridinyl)-1H-benzimidazol-6-yl]methylidene}-2-[(2,6-dichlorophenyl)imino]-1,3-thiazolidin-4-one;

(2Z,5Z)-2-[(2,6-dichlorophenyl)imino]-5-{[1-methyl-2-(3-pyridinyl)-1H-benzimidazol-5-yl]methylidene}-1,3-thiazolidin-4-one;

(2Z,5Z)-5-{[2-(aminomethyl)-1H-benzimidazol-5-yl]methylidene}-2-[(2,6-dichlorophenyl)imino]-1,3-thiazolidin-4-one;

(2Z,5Z)-5-(1H-benzimidazol-5-ylmethylidene)-2-[(2,6-dichlorophenyl)imino]-1,3-thiazolidin-4-one;

(2Z,5Z)-2-[(2,6-dichlorophenyl)imino]-5-{[2-(hydroxymethyl)-1-methyl-1H-benzimidazol-6-yl]methylidene}-1,3-thiazolidin-4-one;

(5Z)-2-[(2,6-dichlorophenyl)amino]-5-({1-[2-(3-pyridinyl)ethyl]-1H-benzimidazol-6-yl}methylidene)-1,3-thiazol-4(5H)-one;

(5Z)-5-{[1-(cyclopropylmethyl)-1H-benzimidazol-6-yl]methylidene}-2-[(2,6-dichlorophenyl)amino]-1,3-thiazol-4(5H)-one;

(5Z) -5-[(1-cyclopentyl-1H-benzimidazol-6-yl)methylidene]-2-[(2,6-dichlorophenyl)amino]-1,3-thiazol-4(5H)-one; and

(5Z)-5-(1,3-Benzoxazol-6-ylmethylidene)-2-[(2,6-dichlorophenyl)amino]-1,3-thiazol-4(5H)-one;

and/or a pharmaceutically acceptable salt, hydrate, solvate or pro-drug thereof.

Compounds of Formula (I) are included in the pharmaceutical compositions of the invention and used in the methods of the invention.

By the term “aryl” as used herein, unless otherwise defined, is meant a cyclic or polycyclic aromatic ring containing from 1 to 14 carbon atoms and optionally containing from one to five heteroatoms, provided that when the number of carbon atoms is 1 the aromatic ring contains at least four heteroatoms, when the number of carbon atoms is 2 the aromatic ring contains at least three heteroatoms, when the number of carbons is 3 the aromatic ring contains at least two heteroatoms and when the number of carbon atoms is 4 the aromatic ring contains at least one heteroatom.

By the term “C₁-C₁₂aryl” as used herein, unless otherwise defined, is meant phenyl, naphthalene, 3,4-methylenedioxyphenyl, pyridine, biphenyl, quinoline, pyrimidine, quinazoline, thiophene, furan, pyrrole, pyrazole, imidazole, tetrazole, 4-fluorophenyl and thiazolyl.

By the term “substituted” as used herein, unless otherwise defined, is meant that the subject chemical moiety has one or more substituents selected from the group consisting of:

—CO₂R²⁰; C₁₋₁₂aryl; —C(O)NHS(O)₂R²⁰; —NHS(O)₂R²⁰; hydroxyalkyl; alkoxy;

—C(O)NR²¹R²²; ═C(NH₂)₂; —C(═O)alkyl; —C(═O)aryl; acyloxy; alkyl optionally substituted with from one to three substituents independently selected from —S(O)₂R²⁰, —NHS(O)₂R²⁰, —NHC(═O)R²¹, —NHC(═O)NR²¹R²², —NHC(═O)OR²¹ and —N═CHNMe₂; C₃₋₁₂cycloalkyl optionally substituted with from one to three substituents independently selected from alkyl;

aryloxy; amino; dialkylamino; N-acylamino; —NHC(═O)R²¹; 3,4-methylenedioxyphenyl;

piperidin; morpholin; piperazin; alkylpiperazin; hydroxyl; —(CH₂)_{g}C(O)OR⁸, —S(O)ᵥR⁸;

nitro; tetrazole; cyano; oxo; halogen; trifluoromethyl; —NHC(═O)N R²¹R²² and —NHC(═O)OR²¹; where g is 0-6; R⁸ is hydrogen, amino or alkyl; R²⁰ is selected from hydrogen, C₁-C₄alkyl optionally substituted with one or two substituents independently selected from C₁₋₂aryl, C₁₋₁₂aryl and trifluoromethyl; and R²¹ and R²² are independently selected from hydrogen, aryl, C₃₋₁₂cycloalkyl, trifluoromethyl, and C₁-C₄alkyl optionally substituted with from one to three substituents independently selected from methoxy, dialkylamino, amino, cycloalkyl, C₁₋₁₂aryl, hydroxy, —CO₂Et and —CO₂H; and v is 0-2.

By the term “alkoxy” as used herein is meant —Oalkyl where alkyl is as described herein including —OCH₃ and —OC(CH₃)₂CH₃.

The term “cycloalkyl” and “C₃₋₁₂cycloalkyl”, and derivatives thereof, as used herein unless otherwise defined, is meant a nonaromatic, unsaturated or saturated, cyclic or polycyclic C₃-C₁₂, optionally containing form 1 to 3 heteroatoms.

Examples of cycloalkyl and substituted cycloalkyl substituents as used herein include: cyclohexyl, 4-hydroxy-cyclohexyl, piperidin, morpholin, piperazin, 2-ethylcyclohexyl, propyl 4-methoxycyclohexyl, 4-methoxycyclohexyl, 4-carboxycyclohexyl, cyclopropyl and cyclopentyl.

By the term “acyloxy” as used herein is meant —OC(O)alkyl where alkyl is as described herein. Examples of acyloxy substituents as used herein include: —OC(O)CH₃, —OC(O)CH(CH₃)₂ and —OC(O)(CH₂)₃CH₃.

By the term “N-acylamino” as used herein is meant —N(H)C(O)alkyl, where alkyl is as described herein. Examples of N-acylamino substituents as used herein include:

—N(H)C(O)CH₃, —N(H)C(O)CH(CH₃)₂ and —N(H)C(O)(CH₂)₃CH₃.

By the term “heteroatom” as used herein is meant oxygen, nitrogen or sulfur.

By the term “halogen” as used herein is meant a substituent selected from bromide, iodide, chloride and fluoride.

By the term “alkyl” and derivatives thereof and in all carbon chains as used herein is meant a linear or branched, saturated or unsaturated hydrocarbon chain, and unless otherwise defined, the carbon chain will contain from 1 to 12 carbon atoms. Examples of alkyl substituents as used herein include: —CH₃, —CH₂—CH₃, —CH₂—CH₂—CH₃, —CH(CH₃)₂, —C(CH₃)₃, —(CH₂)₂C(CH₃)₃, —(CH₂)₂CH(CH₃)₂, —(CH₂)₃—CH₃, —CH₂—CH(CH₃)₂, —CH(CH₃)—CH₂—CH₃, —CH═CH₂, and —C≡C—CH₃.

By the term “treating” and derivatives thereof as used herein, is meant prophylatic and therapeutic therapy.

Compounds of Formula (I) are included in the pharmaceutical compositions of the invention and used in the methods of the invention. Where a —COOH or —OH group is present, pharmaceutically acceptable esters can be employed, for example methyl, ethyl, pivaloyloxymethyl, and the like for —COOH, and acetate maleate and the like for —OH, and those esters known in the art for modifying solubility or hydrolysis characteristics, for use as sustained release or prodrug formulations.

The treatment of anemia in its various forms, as described herein, is accomplished by increasing the production of red blood cells, and/or hemoglobin, and/or hematocrit.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

Because the pharmaceutically active compounds of the present invention are hYAK3 inhibiting compounds they exhibit therapeutic utility in treating anemia and other conditions with depressed red blood cell production.

By the term “anemia” and derivatives thereof as used herein is to be broadly interpreted as any decrease in the number of red blood cells below what is considered normal or desired for a healthy individual. Anemia is known to have many causative factors, including but not limited to, renal insufficiency, chronic disease, such as autoimmunity, HIV, cancer, drug-induced anemias, myelodysplastic syndrome, aplastic anemia, myelosuppression, and cytopenia. The pharmaceutically active compounds of this invention are useful in treating anemia regardless of the factor or factors causing the condition. The pharmaceutically active compounds of this invention are also useful in treating anemia when the causative factor or factors of the condition are unknown or have yet to be identified.

Optimal dosages to be administered may be readily determined by those skilled in the art, and will vary with the particular hYAK3 inhibiting compound in use, the strength of the preparation, the mode of administration, and the advancement of the disease condition. Additional factors depending on the particular patient being treated will result in a need to adjust dosages, including patient age, weight, diet, and time of administration.

Prophylactic use of the compounds of this invention is contemplated whenever a decrease in blood or blood cells is anticipated. Prophylactic use of the compounds of this invention results in a build up of red blood cells or a commencement of red blood cell production prior to an anticipated loss of blood or blood cells. Prophylactic uses of the compounds of this invention includes but is not limited to transplant surgery, surgery, anesthesia prior to child birth and gut protection.

As used herein, the term “optionally” means that the subsequently described event(s) may or may not occur, and includes both event(s), which occur, and events that do not occur.

As used herein, the crisscrossed double bond indicated by the symbol 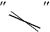
denotes Z and/or E stereochemistry around the double bond. In other words a compound of formula I can be either in the Z or E stereochemistry around this double bond, or a compound of formula I can also be in a mixture of Z and E stereochemistry around the double bond. However, in formula I, the preferred compounds have Z stereochemistry around the double bond to which radical Q is attached.

A compound of formula I naturally may exist in one tautomeric form or in a mixture of tautomeric forms. For example, for sake simplicity, a compound of formula I is expressed in one tautomeric form, usually as an exo form, i.e.

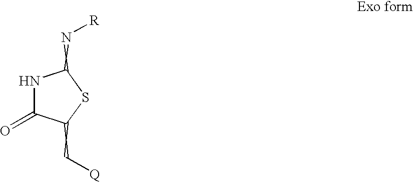

However, a person of ordinary skill can readily appreciate the compounds of formula I can also exist in endo forms.

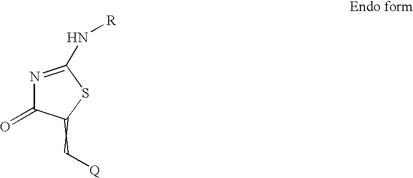

The present invention contemplates all possible tautomeric forms.

Certain compounds described herein may contain one or more chiral atoms, or may otherwise be capable of existing as two enantiomers, or two or more diastereoisomers. Accordingly, the compounds of this invention include mixtures of enantiomers/diastereoisomers as well as purified enantiomers/diastereoisomers or enantiomerically/diastereoisomerically enriched mixtures. Also included within the scope of the invention are the individual isomers of the compounds represented by Formula I above as well as any wholly or partially equilibrated mixtures thereof. The present invention also covers the individual isomers of the compounds represented by the formulas above as mixtures with isomers thereof in which one or more chiral centers are inverted. Also, as stated above, it is understood that all tautomers and mixtures of tautomers are included within the scope of the compounds of Formula I.

While it is possible that, for use in therapy, therapeutically effective amounts of a compound of Formula I, as well as pharmaceutically acceptable salts, hydrates, solvates and pro-drugs thereof, may be administered as the raw chemical, it is possible to present the active ingredient as a pharmaceutical composition. Accordingly, the invention further provides pharmaceutical compositions (otherwise referred to as pharmaceutical formulations), which include therapeutically effective amounts of compounds of the Formula I and pharmaceutically acceptable salts, hydrates, solvates and pro-drugs thereof, and one or more pharmaceutically acceptable carriers, diluents, or excipients. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the Formula I, or a pharmaceutically acceptable salt, hydrate, solvate or pro-drug thereof, with one or more pharmaceutically acceptable carriers, diluents or excipients.

Pharmaceutical formulations may be presented in unit dose forms containing a predetermined amount of active ingredient per unit dose. Such a unit may contain, for example, 0.5 mg to 1 g, suitably 1 mg to 700 mg, suitably 5 mg to 100 mg of a compound of the Formula I, depending on the condition being treated, the route of administration and the age, weight and condition of the patient, or pharmaceutical formulations may be presented in unit dose forms containing a predetermined amount of active ingredient per unit dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The compounds of Formula I, and pharmaceutically acceptable salts, hydrates, solvates and pro-drugs thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of Formula I, and pharmaceutically acceptable salts, hydrates, solvates and pro-drugs thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

A therapeutically effective amount of a compound of the present invention will depend upon a number of factors including, for example, the age and weight of the animal, the precise condition requiring treatment and its severity, the nature of the formulation, and the route of administration, and will ultimately be at the discretion of the attendant physician or veterinarian. However, an effective amount of a compound of Formula I for the treatment of or prevention of diseases of the erythroid and hematopoietic systems, caused by hYAK3 imbalance or inappropriate activity including, but not limited to, neutropenia; cytopenia; anemias, including anemias due to renal insufficiency or to a chronic disease, such as autoimmunity, HIV or cancer, and drug-induced anemias; and myelosuppression will generally be in the range of 0.1 to 100 mg/kg body weight of recipient (mammal) per day and more usually in the range of 1 to 10 mg/kg body weight per day. When treating a human patient in need of increased red blood cell count, the selected dose is administered preferably from 1-6 times daily, orally or parenterally. Preferred forms of parenteral administration include topically, rectally, transdermally, by injection and continuously by infusion. Oral dosage units for human administration preferably contain from 0.05 to 3500 mg of active compound. Oral administration, which uses lower dosages is preferred. Parenteral administration, at high dosages, however, also can be used when safe and convenient for the patient. It is envisaged that similar dosages would be appropriate for treatment of the other conditions referred to above.

By the term “co-administering” and derivatives thereof as used herein is meant either simultaneous administration or any manner of separate sequential administration of a hYAK3 inhibiting compound, as described herein, and a further active ingredient or ingredients, known to treat anemia, including chemotherapy-induced anemia and bone marrow transplantation and other conditions with depressed red blood cell production. The term further active ingredient or ingredients, as used herein, includes EPO, EPO derivatives, any compound or therapeutic agent known to or that demonstrates advantageous properties when administered with hYAK3 inhibiting compound. Preferably, if the administration is not simultaneous, the compounds are administered in a close time proximity to each other. Furthermore, it does not matter if the compounds are administered in the same dosage form, e.g. one compound may be administered topically and another compound may be administered orally.

Examples of a further active ingredient or ingredients for use in combination with the presently invented hYAK3 inhibiting compounds include but are not limited to: EPO and therapeutic agents that increase red blood cell count, and/or hemoglobin, and/or hematocrit.

Method of Preparation

Compounds of general formula I may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula I. Those skilled in the art will recognize if a stereocenter exists in compounds of formula I. Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

More particularly, the compounds of the formula I can be made by the process of either Scheme A or B or a variant thereof. Any person skilled in the art can readily adapt the process of either A or B, such the stoichemistry of the reagents, temperature, solvents, etc. to optimize the yield of the products desired.

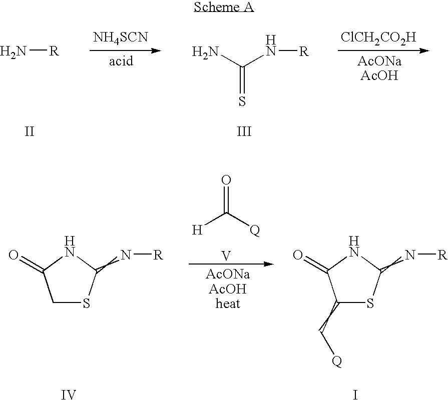

Briefly in Scheme A, a mixture of aniline derivative of formula II (1 equivalent) and NH4SCN (about 1.3 equivalent) in an acid (typically 4N—HCl) is heated to reflux at about 110° C. for 6 hours. After cooling, the mixture is treated with H₂O, which process usually forms a solid, followed by desiccation in vacuo to give a compound of formula III. (However, the compounds of formula III are often commercially available.)

A mixture of formula III compound, ClCH₂CO₂H (1 equivalent), and AcONa (1 equivalent) in AcOH is heated to reflux at around 110° C. for about 4 h. The mixture is poured onto water thereby a solid is typically formed, which is isolated by filtration. The solid is washed with a solvent such as MeOH to afford a compound of formula IV.

A mixture of formula IV compound, an aldehyde of formula V (1 equivalent), AcONa (3 equivalent) in AcOH is heated to reflux at about 110° C. for about 10 to 48 hours. After cooling, a small portion of water was added until the solid forms. The solid is filtered and washed with a solvent such as MeOH, followed by desiccation in vacuo to afford a target product of formula I.

As a variation of Scheme A, a compound of formula IV can also be synthesized according to Scheme A′ or Scheme A″.

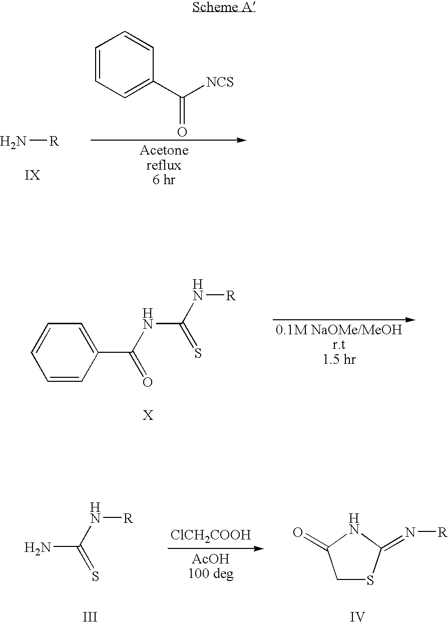

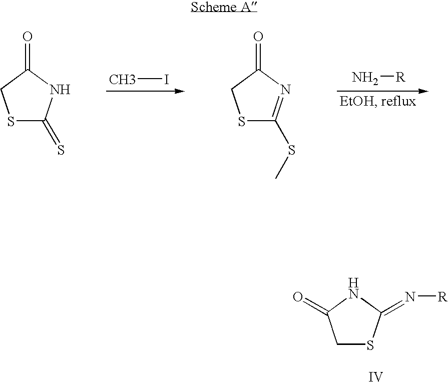

Compounds of formula V are known or can be made by standard organic chemical techniques. For example, Schemes 1 to 11 depict some of the ways to make a compound of formula V, and further ways to make a compound of formula I from a compound of formula V.

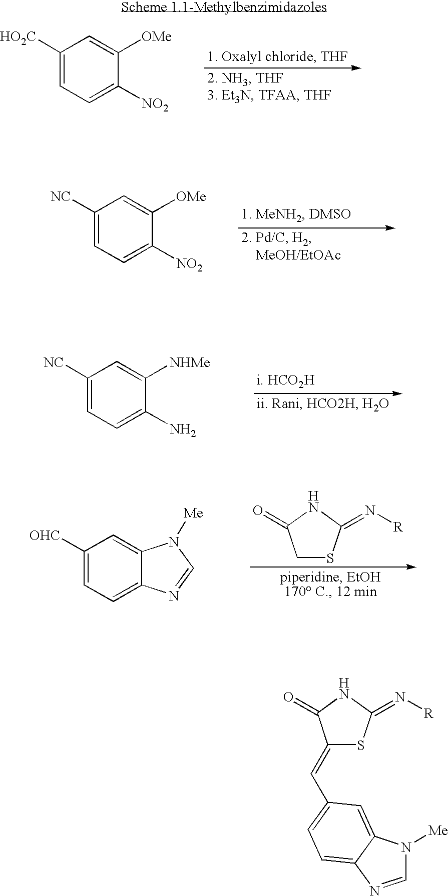

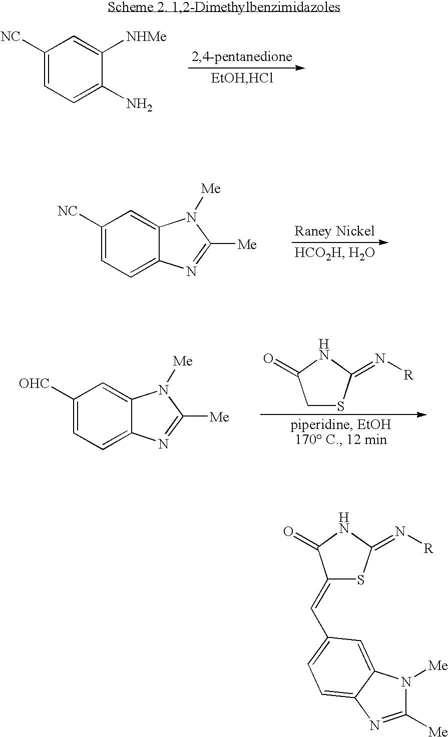

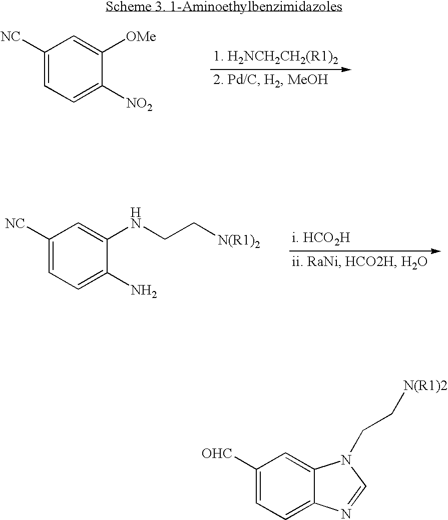

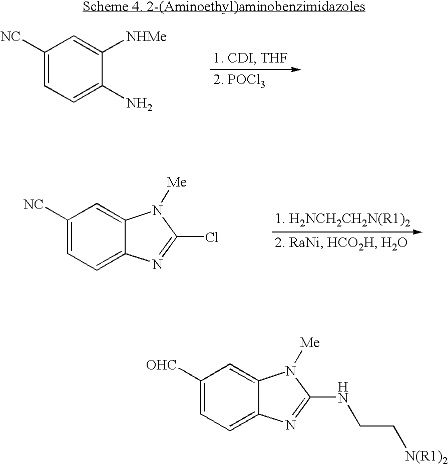

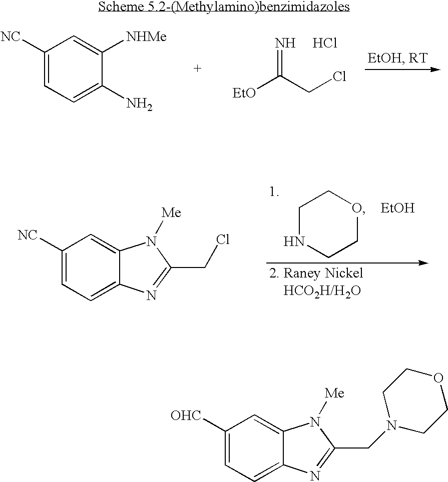

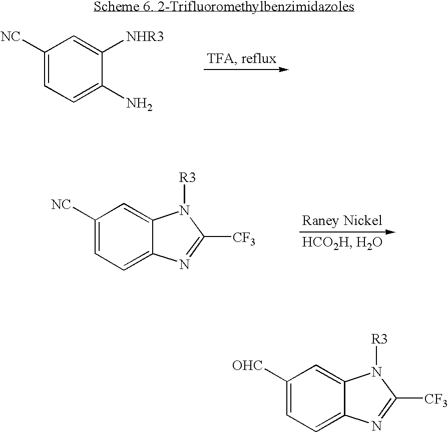

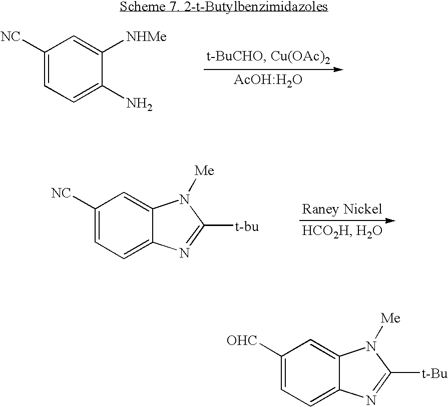

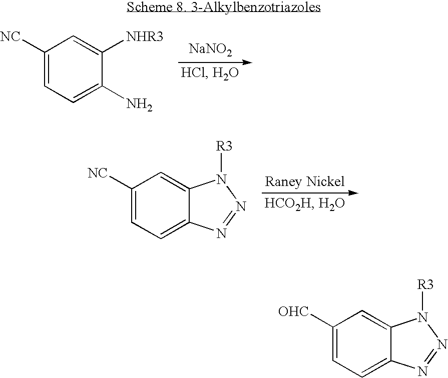

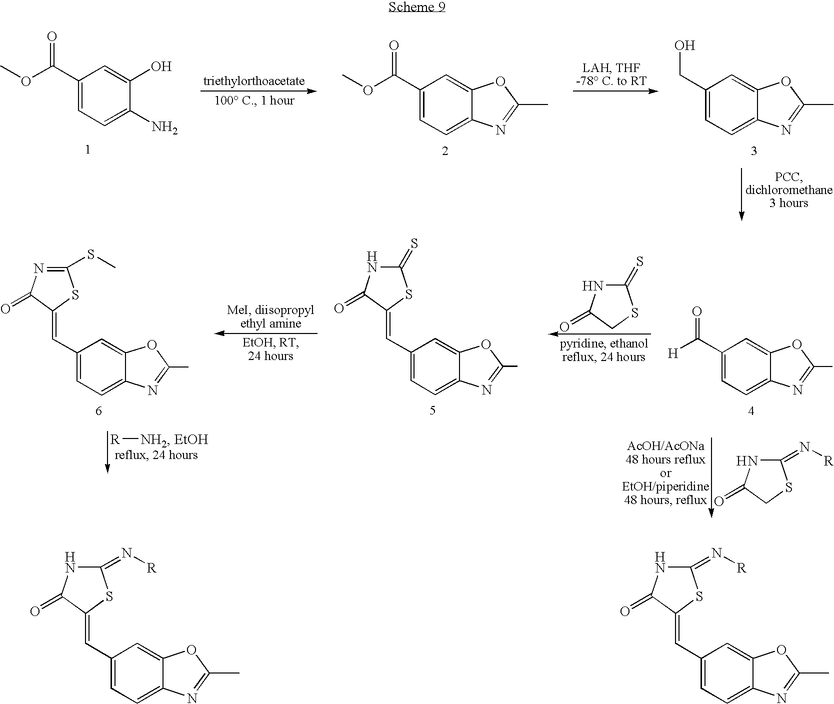

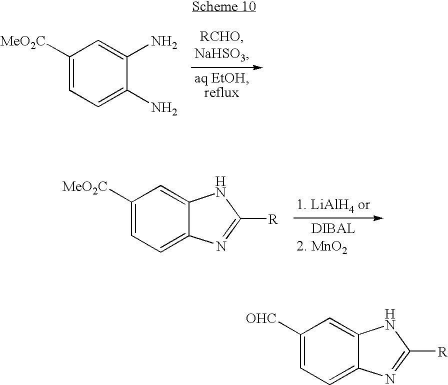

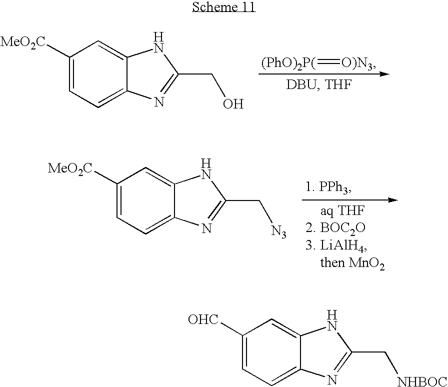

Briefly in Scheme 9, preparation of aldehyde 4 starts with cyclization of methyl 4-amino-3-hydroxy-benzoate 1. Benzoxazole 2 is formed by the reaction with triethylortho acetate. Other reagents, such us, but not limited to, acetamide, acetic anhydride, acetyl chloride, could be utilized in this reaction. Formed benzoxazole is then isolated from the reaction mixture by filtration. Reduction of the ester to the alcohol 3 is done using lithium aluminum hydride. Other reducing agents, such us, but limited to, DIBAL-H, diborane, sodium-ammonia, sodium borohydride can be used for this reaction. Oxidation of alcohol in the presence of PCC yields aldehyde 4. Other oxidative reagents, such us MnO₂ or Swern oxidation can be utilized in this case. Coupling of the aldehyde with thiazolidinone utilizing Knoevenagel reaction can proceed under acid or basis catalysis. When benzoxazole undergoes acid-catalyzed reaction, partial formation of the ring-opening product may be observed. Product is then purified by column chromatography. Coupling with rhodanine under basic conditions yields thiazolidinone 5, which was then methylated with MeI to give thiazolidinone 6. Other methylating agents suitable for this reaction are diazomethane, methyl sulfoxide or other suitable methylating agents. Displacement with a variety of alkyl and aryl amines is done in ethanol and pure product can be isolated by filtration.

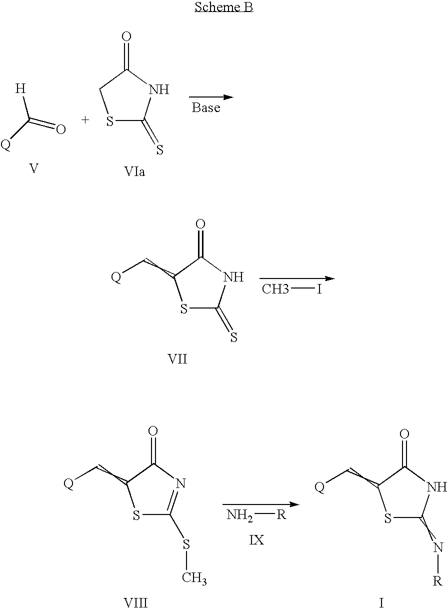

Scheme B is a variant of process of Scheme 9. Briefly in Scheme B, a mixture of an aldehyde of formula V (1 equivalent ), rhodanine (1 equivalent), sodium acetate (about 3 equivalents), and acetic acid is heated at around 110° C. for about 48 h. The reaction mixture is cooled to room temperature to afford a product of formula VII.

Then, to a room temperature suspension of VII (1 equivalent) in a suitable solvent such as ethanol is added Hunig's base (about 2 equivalents) followed by iodomethane (about 5 equivalents). Stirring the resultant suspension at room temperature for 3.5 h will yield a compound of VIII. To a mixture of VIII (1 equivalent) and MS4A powder is added an amine of formula IX (1˜2 equivalent) and ethanol (dehydrated). The mixture was heated to afford a compound of formula I.

In the above Schemes, the meaning of R, R1, R3, and Q are as defined for Formula I.

### Specific Embodiments

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

| | |
|---|---|
| g (grams); | mg (milligrams); |
| L (liters); | mL (milliliters); |
| μL (microliters); | psi (pounds per square inch); |
| M (molar); | mM (millimolar); |
| i.v. (intravenous); | Hz (Hertz); |
| MHz (megahertz); | mol (moles); |
| mmol (millimoles); | rt (room temperature); |
| min (minutes); | h (hours); |
| mp (melting point); | TLC (thin layer chromatography); |
| Tr (retention time); | RP (reverse phase); |
| MeOH (methanol); | i-PrOH (isopropanol); |
| TEA (triethylamine); | TFA (trifluoroacetic acid); |
| TFAA (trifluoroacetic anhydride); | THF (tetrahydrofuran); |
| DMSO (dimethylsulfoxide); | AcOEt (ethyl acetate); |
| DME (1,2-dimethoxyethane); | DCM (dichloromethane); |
| DCE (dichloroethane); | DMF (N,N-dimethylformamide); |
| DMPU (N,N′-dimethylpropyleneurea); | (CDI (1,1-carbonyldiimidazole); |
| IBCF (isobutyl chloroformate); | HOAc (acetic acid); |
| HOSu (N-hydroxysuccinimide); | HOBT (1-hydroxybenzotriazole); |
| mCPBA (meta-chloroperbenzoic acid; | EDC (ethylcarbodiimide hydrochloride); |
| BOC (tert-butyloxycarbonyl); | FMOC (9-fluorenylmethoxycarbonyl); |
| DCC (dicyclohexylcarbodiimide); | CBZ (benzyloxycarbonyl); |
| Ac (acetyl); | atm (atmosphere); |
| TMSE (2-(trimethylsilyl)ethyl); | TMS (trimethylsilyl); |
| TIPS (triisopropylsilyl); | TBS (t-butyldimethylsilyl); |
| DMAP (4-dimethylaminopyridine); | BSA (bovine serum albumin) |
| ATP (adenosine triphosphate); | HRP (horseradish peroxidase); |
| DMEM (Dulbecco's modified Eagle medium); | |
| HPLC (high pressure liquid chromatography); | |
| BOP (bis(2-oxo-3-oxazolidinyl)phosphinic chloride); | |
| TBAF (tetra-n-butylammonium fluoride); | |
| HBTU (O-Benzotriazole-1-yl-N,N,N′,N′- | |
| tetramethyluronium hexafluorophosphate). | |
| HEPES (4-(2-hydroxyethyl)-1- | |
| piperazine ethane sulfonic acid); | |
| DPPA (diphenylphosphoryl azide); | |
| fHNO3 (fumed HNO3); and | |
| EDTA (ethylenediaminetetraacetic acid). | |

All references to ether are to diethyl ether; brine refers to a saturated aqueous solution of NaCl. Unless otherwise indicated, all temperatures are expressed in ° C. (degrees Centigrade). All reactions are conducted under an inert atmosphere at room temperature unless otherwise noted.

¹H NMR spectra were recorded on a Varian VXR-300, a Varian Unity-300, a Varian Unity-400 instrument, a Brucker AVANCE-400, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), quint (quintet), m (multiplet), br (broad).

Low-resolution mass spectra (MS) were recorded on a JOEL JMS-AX505HA, JOEL SX-102, or a SCIEX-APIiii spectrometer; LC-MS were recorded on a micromass 2MD and Waters 2690; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (IR) spectra were obtained on a Nicolet 510 FT-IR spectrometer using a 1-mm NaCl cell. Most of the reactions were monitored by thin-layer chromatography on 0.25 mm E. Merck silica gel plates (60F-254), visualized with UV light, 5% ethanolic phosphomolybdic acid or p-anisaldehyde solution. Flash column chromatography was performed on silica gel (230-400 mesh, Merck).

In the present specification, often the regiochemistry around the double bonds in the chemical formulas are drawn as fixed for ease of representation; however, a skilled in the art will readily appreciate that the compounds will naturally assume more thermodynamically stable structure around the C═N (the imine) double bond, if they exit, as exo form. Further compounds can also exit in endo form. As stated before, the invention contemplates both endo and exo forms as well as both regioisomers around the exo imine bond. Further it is intended that both E and Z isomers are encompassed around the C═C double bond.

The method of this invention of inducing hYAK3 inhibiting activity in mammals, including humans, comprises administering to a subject in need of such activity an effective hYAK3 inhibiting amount of a pharmaceutically active compound of the present invention.

The invention also provides for the use of a compound of Formula (I) in the manufacture of a medicament for use as an inhibitor of hYAK3.

The invention also provides for the use of a compound of Formula (I) in the manufacture of a medicament for use in therapy.

The invention also provides for the use of a compound of Formula (I) in the manufacture of a medicament for use in enhancing red blood cell production.

The invention also provides for the use of a compound of Formula (I) in the manufacture of a medicament for use in treating anemia.

The invention also provides for a pharmaceutical composition for use in the inhibition of hYAK3 which comprises a compound of Formula (I) and a pharmaceutically acceptable carrier.

The invention also provides for a pharmaceutical composition for use in the treatment of anemia which comprises a compound of Formula (I) and a pharmaceutically acceptable carrier.

The invention also provides for a pharmaceutical composition for use in enhancing red blood cell production which comprises a compound of Formula (I) and a pharmaceutically acceptable carrier.

No unacceptable toxicological effects are expected when compounds of the invention are administered in accordance with the present invention.

In addition, the pharmaceutically active compounds of the present invention can be co-administered with further active ingredients, such as other compounds known to treat anemia, including chemotherapy-induced anemia and bone marrow transplantation and other conditions with depressed red blood cell production, or compounds known or found to have utility when used in combination with a hYAK3 inhibiting compound.

Without further elaboration, it is believed that one skilled in the art can, using the preceding description, utilize the present invention to its fullest extent. The following Examples are, therefore, to be construed as merely illustrative and not a limitation of the scope of the present invention in any way.

### Example 1

### (5Z)-2-[(2-Chlorophenyl)amino]-5-[(1-methyl-1H-benzimidazol-6-yl)methylidene]-1,3-thiazol-4(5H)-one

(a) 3-(Methyloxy)-4-nitrobenzonitrile. Following the procedure of Mackman et al. in J. Med. Chem. 2001, 44, 2753-2771, 3-methoxy-4-nitrobenzoic acid (11.52 g, 58.4 mmol) was dissolved in THF (158 mL) and cooled to 0° C. Oxalyl chloride (5.6 mL, 64.3 mmol) was added dropwise under a nitrogen atmosphere, followed by a few drops of DMF. The reaction mixture was allowed to warm to room temperature and stirred for 1 h. The mixture was then concentrated to dryness under reduced pressure, and the residue redissolved in THF (158 mL) and cooled to 0° C. Ammonia gas was bubbled through the solution for 10 min, leading to the formation of a yellow precipitate. The ice bath was removed, and the mixture was sealed and allowed to stir overnight. After the addition of EtOAc (100 mL), the solids were filtered off, washed with water, and dried to provide 3-(methyloxy)-4-nitrobenzamide (10.10 g, 88%) as a yellow solid. Additional product could be recovered from the filtrate by removal of the organic solvent under reduced pressure, then redissolving the residue in EtOAc. The organic layer was washed with 1N HCl (2×100 mL), brine (2×100 mL), then dried (Na₂SO₄), filtered and concentrated to afford an additional 1.07 g (9%). ¹H NMR (d₆-DMSO): δ 8.25 (bs, 1H), 7.96 (d, J=8.0 Hz, 1H), 7.75 (d, J=1.6 Hz, 1H), 7.73 (s, 1), 7.57 (dd, J=1.6, 8.4 Hz, 1H), 3.98 (s, 3H).

To a suspension of 3-(methyloxy)-4-nitrobenzamide (11.17 g, 56.7 mmol) in THF (150 mL) was added Et₃N (10.3 mL, 73.7 mmol), followed by the dropwise addition of TFAA (8.67 mL, 62.4 mmol). After stirring for 1.5 h, the solvent was removed in vacuo and the mixture dissolved in EtOAc (400 mL). The solution was washed with 1N HCl (1×200 mL), brine (2×250 mL), dried over Na₂SO₄, filtered and concentrated to yield 3-(methyloxy)-4-nitrobenzonitrile (9.98 g, 96% overall) as a yellow solid. ¹H NMR (d₆-DMSO): δ 8.06 (d, J=8.4 Hz, 1H), 7.96 (d, J=1.2 Hz, 1H), 7.64 (dd, J=1.2, 8.4 Hz, 1H), 3.98 (s, 3H).

(b) 3-(Methylamino)-4-nitrobenzonitrile. Following the procedure of Mackman et al. in J. Med. Chem. 2001, 44, 2753-2771, 3-methoxy-4-nitrobenzonitrile (1.0 g, 5.62 mmol) was dissolved in DMSO (7 mL) in a pressure tube and a 40% solution of MeNH₂ in water (1 mL) was added. The tube was sealed and heated to 75° C. for 4 h, then cooled and poured onto an ice/water mixture. The precipitate was filtered, rinsed with water, and dried to afford 3-(methylamino)-4-nitrobenzonitrile (0.95 g, 95%) as an orange solid. ¹H NMR (CDCl₃): δ 8.26 (d, J=8.8 Hz, 1H), 8.05 (bs, 1H), 7.15 (d, J=1.2 Hz, 1H), 6.89 (dd, J=1.6, 8.8 Hz, 1H), 3.06 (d, J=5.2 Hz, 3H).

(c) 4-Amino-3-(methylamino)benzonitrile. To a mixture of 3-(methylamino)-4-nitrobenzonitrile (0.655 g, 3.70 mmol) in MeOH (9.5 mL) and EtOAc (9.5 mL) was added 10% Pd/C (65 mg). After stirring under a hydrogen atmosphere for 4 h, the reaction mixture was filtered through a pad of Celite, rinsed with MeOH, and concentrated under reduced pressure to afford 4-amino-3-(methylamino)benzonitrile (0.542 g, 100%) as a beige solid. ¹H NMR (CDCl₃): δ 7.02 (dd, J=1.6, 8.0 Hz, 1H), 6.84 (d, J=1.2 Hz, 1H), 6.68 (d, J=8.0 Hz, 1H), 3.74 (bs, 2H), 3.32 (bs, 1H), 2.87 (s, 3H).

(d) 1-Methyl-1H-benzimidazole-6-carbaldehyde. A mixture of 4-amino-3-(methylamino)benzonitrile (0.40 g, 2.72 mmol) in HCO₂H (9 mL) was heated to 100° C. for 2 h. The mixture of crude benzimidazole was then cooled, Raney nickel (0.4 g) and H₂O (2 mL) were added, and the mixture was heated again to 100° C. for 1 h. The hot mixture was then filtered through Celite, rinsed with MeOH and concentrated under reduced pressure. Water (1 mL) was added to the residue, which was then treated carefully with sat. aq. NaHCO₃. The solid which precipitated was filtered, rinsed with H₂O and dried to afford 1-methyl-1H-benzimidazole-6-carbaldehyde (0.412 g, 95%) as a tan solid, which was used directly in the next reaction. ¹H NMR (CDCl₃): δ 10.12 (s, 1H), 8.05 (s, 1H), 8.00 (d, J=0.8 Hz, 1H), 7.92 (d, J=8.0 Hz, 1H), 7.84 (dd, J=1.2, 8.0 Hz, 1H), 3.94 (s, 3H).

(e)(5Z)-2-[(2-Chlorophenyl)amino]-5-[(1-methyl-1H-benzimidazol-6-yl)methylidene]-1,3-thiazol-4(5H)-one. A solution of 1-methyl-1H-benzimidazole-6-carbaldehyde (15 mg, 0.094 mmol), 2-[(2-chlorophenyl)amino]-1,3-thiazol-4(5H)-one (21.3 mg, 0.094 mmol), and piperidine (18.5 μL, 0.188 mmol) in EtOH (0.5 mL) was heated in a microwave reactor at 170° C. for 720 s. The solvent was then removed under reduced pressure and the crude product purified by precipitation from a mixture of CH₂Cl₂/hexanes. Alternatively, the product could be purified by column chromatography.

| Example | Compound name | NMR(400MHz) |
|---|---|---|
| 1 | (5Z)-2-[(2- | (CDCl₃): 7.95(s, 1H), 7.92(s, 1H), 7.81(d, J=8.8Hz, |
| | Chlorophenyl)amino]- | 1H), 7.48(m, 2H), 7.39(dd, J=0.8, 8.8Hz, |
| | 5-[(1-methyl-1H- | 1H), 7.31(dt, J=1.6, 7.6Hz, 1H), 7.17(dt, |
| | benzimidazol-6- | J=1.6, 7.6Hz, 1H), 7.08(m, 1H), 3.86(s, 3H) |
| | yl)methylidene]- | |
| | 1,3-thiazol- | |
| | 4(5H)-one | |

### Examples 2-8

The following compounds were prepared according to the procedure of Example 1, except substituting the appropriately substituted thiazolone for 2-[(2-chlorophenyl)amino]-1,3-thiazol-4(5H)-one:

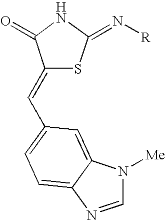

| Example | Compound name | R | NMR (400 MHz) |
|---|---|---|---|
| 2 | (5Z)-2-[(2,6-dichlorophenyl)- amino]-5-[(1-methyl-1H- benzimidazol-6-yl)- methylidene]-1,3-thiazol- 4(5H)-one | 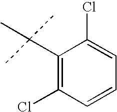 | (d₆-acetone): 8.11(s, 1H), 7.70 (m, 3H), 7.43(m, 3H), 7.12(m, 1H), 3.91(s, 3H) |
| 3 | (5Z)-2-[(2,6-diflurophenyl)- amino]-5-[(1-methyl-1H- benzimidazol-6-yl)- methylidene]-1,3-thiazol- 4(5H)-one | 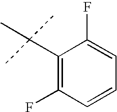 | (CDCl₃): 7.95(s, 1H), 7.92(s, 1H), 7.81 (d, J=8.0Hz, 1H), 7.46(s, 1H), 7.38(m, 1H), 7.12 (m, 1H), 7.00(t, J=7.6Hz, 2H), 3.89(s, 3H) |
| 4 | (5Z)-2-[(2,6-dimethylphenyl)- amino]-5-[(1-methyl-1H- benzimidazol-6-yl)- methylidene]-1,3-thiazol- 4(5H)-one | 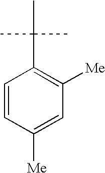 | (CDCl₃): 7.93(s, 1H), 7.90(s, 1H), 7.80(d, J=7.6Hz, 1H), 7.45(s, 1H), 7.39(dd, J=0.8, 8.8Hz, 1H), 7.10(s, 1H), 7.05 (dd, J=2.0, 8.0Hz, 1H), 6.91 (d, J=8.8Hz, 1H), 3.89(s, 3H), 2.36(s, 3H), 2.22(s, 3H) |
| 5 | (5Z)-5-[(1-methyl-1H- benzimidazol-6-yl)- methylidene]-2-{[2- (methyloxy)- phenyl]amino}-1,3- thiazol-4(5H)-one | 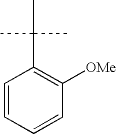 | (CDCl₃): 7.95(s, 1H), 7.84(m, 1H), 7.51(m, 2H), 6.99(m, 2H), 3.91(s, 3H), 3.89(s, 3H) |
| 6 | (5Z)-5-[(1-methyl-1H- benzimidazol-6-yl)- methylidene]-2-{[2- (trifluoromethyl)- phenyl]amino}-1,3- thiazol-4(5H)-one | 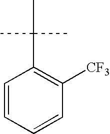 | (CDCl₃): 7.94(s, 1H), 7.89(s, 1H), 7.80(dd, J=0.8, 8.4Hz, 1H), 7.71(d, J=7.6Hz, 1H), 7.56(m, 2H), 7.45(m, 1H), 7.38 (m, 1H), 7.10(m, 1H), 3.85(s, 3H) |
| 7 | (5Z)-2-[(2,4-difluorophenyl)- amino]-5-[(1-methyl-1H- benzimidazol-6-yl)- methylidene]-1,3-thiazol- 4(5H)-one | 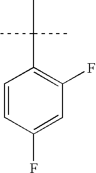 | (CDCl₃): 7.95(s, 1H), 7.88(m, 1H), 7.43(m, 1H), 7.35(m, 2H), 6.92(m, 2H), 3.74(s, 3H), 2.63 (s, 3H) |
| 8 | (5Z)-2-[(2-chloro-4-fluorophenyl)- amino]-5-[(1-methyl-1H- benzimidazol-6-yl)- methylidene]-1,3-thiazol- 4(5H)-one | 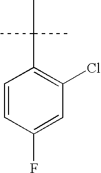 | (CD₃OD): 9.33(s, 1H), 8.01(s, 1H), 7.88(m, 2H), 7.74(dd, J=1.6, 8.4Hz, 1H), 7.33(d, J=8.4Hz, 1H), 7.11(d, J=6.4Hz, 2H), 4.11(s, 3H) |

### Example 9

### (5Z)-2-[(2-Chlorophenyl)amino]-5-[(1,2-dimethyl-1H-benzimidazol-6-yl)methylidene]-1.3-thiazol-4(5H)-one

(a) 1,2-Dimethyl-1H-benzimidazole-6-carbonitrile. 4-Amino-3-(methylamino)benzonitrile (from Example 1(c); 0.500 g, 3.40 mmol) and 2,4-pentanedione (0.700 mL, 6.80 mmol) were dissolved in EtOH (8.4 mL) and cooled to 0° C. 6N HCl (2.8 mL) was added dropwise and the solution turned deep red. Stirring was continued for 20 min, after which time the mixture was carefully poured onto ice/sat. aq. NaHCO₃, making sure the aqueous layer remained basic. The solid product which precipitated was filtered off, rinsed with H₂O and dried to afford the crude 1,2-dimethyl-1H-benzimidazole-6-carbonitrile (0.355 g, 61%). ¹H NMR (CDCl₃): δ 7.72 (d, J=8.4 Hz, 1H), 7.62 (d, J=1.2 Hz, 1H), 7.50 (dd, J=1.2, 8.4 Hz, 1H), 3.78 (s, 3H), 2.66 (s, 3H).

(b) 1,2-Dimethyl-1H-benzimidazole-6-carbaldehyde. A mixture of 1,2-dimethyl-1H-benzimidazole-6-carbonitrile (0.355 g, 2.08 mmol) and Raney nickel (150 mg) was suspended in HCO₂H (7 mL) and H₂0 (3 mL) and heated to 100° C. for 2 h. The mixture was then filtered hot through Celite, rinsed with MeOH, and concentrated. To the resulting residue was added H₂O (1 mL) followed by sat. aq. NaHCO₃ until basic. The aqueous layer was extracted with CH₂Cl₂ (2×30 mL), dried over Na₂SO₄, and concentrated to yield the product aldehyde (0.284 g, 79%) as a beige solid. ¹H NMR (CDCl₃): δ 10.07 (s, 1H), 7.88 (t, J 1.2 H, 1H), 7.77 (d, J=1.2 Hz, 2H), 3.81 (s, 3H), 2.61 (s, 3H).

(c)(5Z)-2-[(2-Chlorophenyl)amino]-5-[(1,2-dimethyl-1H-benzimidazol-6-yl)methylidene]-1,3-thiazol-4(5H)-one. Knoevenagel coupling with 2-[(2-chlorophenyl)amino]-1,3-thiazol-4(5H)-one was accomplished using the procedure of Example 1(e).

| Example | Compound name | NMR(400MHz) |
|---|---|---|
| 9 | (5Z)-2-[(2-Chlorophenyl)- | (d₆-DMSO): 12.58(s, 1H), |
| | amino]-5- | 7.81(s, 1H), 7.72(s, 1H), 7.59(d, |
| | [(1,2-dimethyl-1H- | J=8.4Hz, 1H), 7.55(d, J=8.0Hz, |
| | benzimidazol-6-yl)- | 1H), 7.38(m, 1H), 7.23(m, 3H), |
| | methylidene]- | 3.72(s, 3H), 2.53(s, 3H) |
| | 1,3-thiazol-4(5H)-one | |

### Examples 10-14

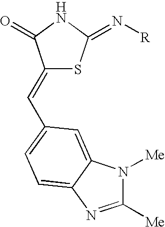

The following compounds were prepared according to the procedure of Example 9, except substituting the appropriate thiazolone for 2-[(2-chlorophenyl)amino]-1,3-thiazol-4(5H)-one:

| Example | Compound name | R | NMR (400 MHz) |
|---|---|---|---|
| 10 | (5Z)-2-[(2,6-dichlorophenyl)- amino]-5-[(1,2-dimethyl-1H- benzimidazol-6-yl)- methylidene]-1,3-thiazol- 4(5H)-one | 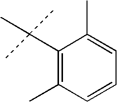 | (d₆-DMSO): 7.57(s, 1H), 7.52 (d, J=7.6Hz, 1H), 7.41(m, 2H), 7.26(m, 1H), 6.97(m, 1H), 3.29(s, 3H), 2.5(s, 3H) |
| 11 | (5Z)-2-[(2,6-difluorophenyl)- amino]-5-[(1,2-dimethyl-1H- benzimidazol-6-yl)- methylidene]-1,3-thiazol- 4(5H)-one | 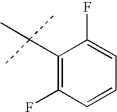 | (CD₃OD): 7.85(s, 1H), 7.56(m, 2H), 7.33(dd, J=1.2, 8.4Hz, 1H), 7.20(m, 1H), 7.05(t, J=8.0Hz, 2H), 3.76(s, 3H), 2.59 (s, 3H) |
| 12 | (5Z)-5-[(1,2-dimethyl-1H- benzimidazol-6-yl)- methylidene]-2- [(2,4-dimethylphenyl)amino]- 1,3-thiazol-4(5H)-one | 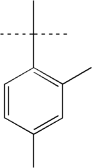 | (d₆-DMSO): 7.76(d, J=3.6Hz, 1H), 7.71(s, 1H), 7.58(d, J=8.4Hz, 1H), 7.22(dd, J=1.6, 8.4Hz, 1H), 7.10(s, 1H), 7.02 (m, 1H), 3.71(s, 3H), 3.29(s, 3H), 2.53(s, 3H), 2.29(s, 3H) |
| 13 | (5Z)-2-[(2,4-difluorophenyl)- amino]-5-[(1,2-dimethyl-1H- benzimidazol-6-yl)- methylidene]-1,3-thiazol- 4(5H)-one | 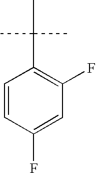 | (CD₃OD): 7.83(s, 1H), 7.58(m, 2H), 7.36(m, 1H), 7.06(m, 3H), 3.77(s, 3H), 2.60(s, 3H) |
| 14 | (5Z)-2-[(2-chloro-4-fluorophenyl)- amino]-5-[(1,2-dimethyl-1H- benzimidazol-6-yl)- methylidene]-1,3-thiazol- 4(5H)-one | 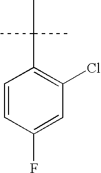 | (CD₃OD): 7.82(s, 1H), 7.57(m, 2H), 7.35(dd, J=0.8, 8.8Hz, 1H), 7.31(d, J=8.8hz, 1H), 7.12(d, J=5.6Hz, 2H), 3.77(s, 3H), 2.60(s, 3H) |

### Example 15

### (5Z)-2-[(2-Chlorophenyl)amino]-5-({1-[2-(4-morpholinyl)ethyl]-1H-benzimidazol-6-yl}methylidene)-1,3-thiazol-4(5H)-one

(a) 4-Amino-3-{[2-(4-morpholinyl)ethyl]amino}benzonitrile. A mixture of 3-methoxy-4-nitrobenzonitrile (from Example 1(a); 0.178 g, 1.0 mmol) and 4-(2-aminoethyl)morpholine (0.65 mL, 5.0 mmol) were heated to 80° C. for 20 h. The reaction mixture was cooled, diluted with EtOAc (50 mL), and washed with H₂O (3×30 mL) and brine (1×30 mL). The organic layer was dried (Na₂SO₄), filtered, and concentrated to afford 3-{[2-(4-morpholinyl)ethyl]amino}-4-nitrobenzonitrile (0.246 g, 89%) as a red solid. This material was dissolved in MeOH (3 mL), 10% Pd/C (24 mg) was added, and the mixture stirred under a hydrogen atmosphere overnight. Filtration through Celite and removal of the solvent provided the desired diaminobenzonitrile (0.219 g, 100%) as a red oil.

(b) 1-[2-(4-Morpholinyl)ethyl]-1H-benzimidazole-6-carbaldehyde. According to the procedure in Example 1(d), 4-amino-3-{[2-(4-morpholinyl)ethyl]amino}benzonitrile was converted to the benzimidazole carboxaldehyde in 72% yield. ¹H NMR (CDCl₃): δ 10.11 (s, 1H), 8.20 (s, 1H), 8.02 (d, J=0.8 Hz, 1H), 7.92 (d, J=8.4 Hz, 1H), 7.82 (dd, J=1.2, 8.4 Hz, 1H), 4.34 (t, J=6.0 Hz, 2H), 3.69 (t, J=4.4Hz, 4H), 2.98 (t, J=6.0 Hz, 2H), 2.50 (t, J=4.8 Hz, 4H).

(c)(5Z)-2-[(2-Chlorophenyl)amino]-5-({1-[2-(4-morpholinyl)ethyl]-1H-benzimidazol-6-yl}methylidene)-1,3-thiazol-4(5H)-one. Knoevenagel coupling with 2-[(2-chlorophenyl)amino]-1,3-thiazol-4(5H)-one was accomplished using the procedure of Example 1(e).

| Example | Compound name | NMR(400MHz) |
|---|---|---|
| 15 | (5Z)-2-[(2-Chlorophenyl)- | (CD₃OD): 8.30(s, 1H), 7.84(s, 1H), 7.72(m, 2H), |
| | amino]-5- | 7.48(dd, J=1.6, 8.0Hz, 1H), 7.41(m, 1H), |
| | ({1-[2-(4-morpholinyl)ethyl]- | 7.33(dt, J=1.6, 7.6Hz, 1H), 7.19(dt, J=1.2, 7.6Hz, |
| | 1H-benzimidazol-6-yl}- | 1H), 7.09(m, 1H), 4.39(t, J=6.4Hz, 2H), |
| | methylidene)-1,3-thiazol- | 3.59(m, 4H), 2.74(t, J=6.4Hz, 2H), 2.43(m, 4H) |
| | 4(5H)-one | |

### Examples 16-32

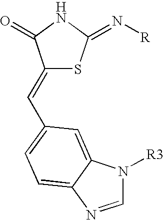

The following compounds were prepared according to the procedure of Example 15, using the requisite amine for 4-(2-aminoethyl)morpholine and substituting the appropriate thiazolone for 2-[(2-chlorophenyl)amino]-1,3-thiazol-4(5H)-one:

| Example | Compound name | R3 | R | NMR (400 MHz) |
|---|---|---|---|---|
| 16 | (5Z)-2-[(2,6-dichlorophenyl)- amino]-5-({1-[2-(4- morpholinyl)ethyl]- 1H-benzimidazol-6-yl}- methylidene)-1,3- thiazol-4(5H)-one | 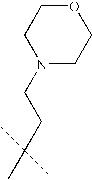 | 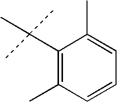 | (CD₃OD): 8.30(s, 1H), 7.87(s, 1H), 7.72(m, 2H), 7.44(d, J=8.0Hz, 2H), 7.39(d, J=8.0Hz, 1H), 7.15(m, 1H), 4.39(t, J= |
| #5.6Hz, 2H), 3.60(m, 4H), 2.74(t, J=5.6Hz, 2H), 2.44(m, 4H) | | | | |
| 17 | (5Z)-2-[(2-chloro-4-fluorophenyl)- amino]-5-({1-[2-(4- morpholinyl)ethyl]- 1H-benzimidazol-6-yl}- methylidene)-1,3- thiazol-4(5H)-one | 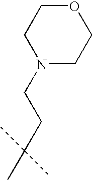 | 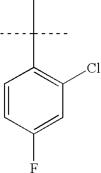 | (CD₃OD): 8.30(s, 1H), 7.85(s, 1H), 7.72(m, 2H), 7.41(dd, J=1.2, 7.6Hz, 1H), 7.33(dd, J=1.2, 9.2Hz, 1H), 7.12(d, J= |
| #7.2Hz, 2H), 4.40(t, J=6.4Hz, 2H), 3.59 (m, 4H), 2.75(t, J=6.0Hz, 2H), 2.44(m, 4H) | | | | |
| 18 | (5Z)-2-[(2-chlorophenyl)- amino]-5-({1-[2- (dimethylamino)- ethyl]-1H-benzimidazol-6-yl}- methylidene)-1,3- thiazol-4(5H)-one | 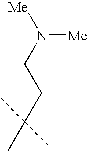 | 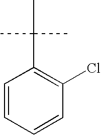 | (CD₃OD): 8.28(s, 1H), 7.84(s, 1H), 7.74(s, 1H), 7.70(d, J=8.4Hz, 1H), 7.47(d, J=8.4Hz, 1H), 7.41(d, J= |
| #8.4Hz, 1H), 7.32(t, J=7.6Hz, 1H), 7.18(t, J=7.6Hz, 1H), 7.09(d, J=7.6Hz, 1H), 4.37 (t, J=6.8Hz, 2H), 2.73(t, J=6.8Hz, 2H) | | | | |
| 19 | (5Z)-2-[(2,6-dichlorophenyl)- amino]-5-({1-[2- (dimethylamino)- ethyl]-1H-benzimidazol-6-yl}- methylidene)-1,3- thiazol-4(5H)-one | 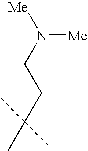 | 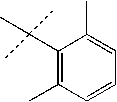 | (CD₃OD): 8.24(s, 1H), 7.71(m, 3H), 7.40(m, 3H), 7.10(t, J=8.0Hz, 1H), 4.35(t, J=6.4Hz, 2H), 2.71(t, J=6.4Hz, 2H), 2.19(s, 6H). |
| 20 | (5Z)-2-[(2,4-difluorophenyl)- amino]-5-({1-[2- (dimethylamino)- ethyl]-1H-benzimidazol-6-yl}- methylidene)-1,3- thiazol-4(5H)-one | 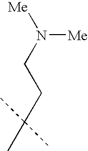 | 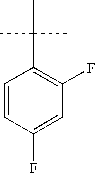 | (CD₃OD): 8.30(s, 1H), 7.84(s, 1H), 7.74(s, 1H), 7.71(d, J=8.4Hz, 1H), 7.43(dd, J=1.2, 8.8Hz, 1H), 7.10(m, 2H), 6.98(m, 1H), 4.39(t, J= |
| #6.8Hz, 2H), 2.75(t, J=6.8Hz, 2H), 2.23(s, 6H) | | | | |
| 21 | (5Z)-5-({1-[2-(dimethylamino)- ethyl]-1H-benzimidazol-6-yl}- methylidene)-2-(phenylamino)- 1,3-thiazol-4(5H)-one | 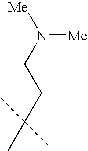 | 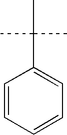 | (CD₃OD, mixture of two isomers): 8.35(s, 1H), 8.29(s, 1H), 7.98(s, 1H), 7.70-7.88(m, 8H), 7.57(m, 1H), 7.41(m, 5H), 7.23(m, 2H), 7.10(d, J=7.6Hz, 2H), 4.48(m, 2H), 4.38(m, 2H), 2.86(m, 2H), |
| #2.74(m, 2H), 2.35(s, 6H), 2.22(s, 6H) | | | | |
| 22 | (5Z)-2-[(2-chlorophenyl)- amino]-5-({1-[2- (diethylamino)ethyl]-1H- benzimidazol-6-yl}- methylidene)- 1,3-thiazol-4(5H)-one | 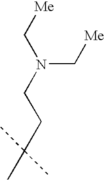 | 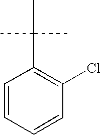 | (CD₃OD): 8.27(s, 1H), 7.84(s, 1H), 7.71(m, 2H), 7.48(dd, J=1.6, 8.0Hz, 1H), 7.41(d, J=8.4Hz, 1H), 7.32(t, J=8.0Hz, 1H), 7.18(t, J=7.6Hz, 1H), 7.08(dd, J= |
| #0.8, 8.0Hz, 1H), 4.32(t, J=6.0Hz, 2H), 2.80(t, J=6.4Hz, 2H), 2.48(q, J=7.2Hz, 4H), 0.86(t, J=7.2Hz, 6H) | | | | |
| 23 | (5Z)-2-[(2,6-dichlorophenyl)- amino]-5-({1-[2- (diethylamino)ethyl]- 1H-benzimidazol-6-yl}- methylidene)-1,3- thiazol-4(5H)-one | 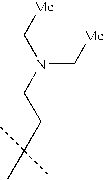 | 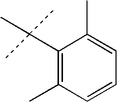 | (CD₃OD): 8.27(s, 1H), 7.85(s, 1H), 7.70(m, 2H), 7.43(d, J=8.0Hz, 2H), 7.40(d, J=8.8Hz, 1H), 7.14(t, J=8.0Hz, 1H), 4.32(t, J=6.4Hz, 2H), 2.80(t, J= |
| #6.4Hz, 2H), 2.47(q, J=6.8Hz, 4H), 0.86 (t, J=6.8Hz, 6H) | | | | |
| 24 | (5Z)-2-[(2-chlorophenyl)- amino]-5-({1-[3-(4- (morpholinyl)propyl]-1H- benzimidazol-6-yl}- methylidene)- 1,3-thiazol-4(5H)-one | 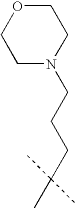 | 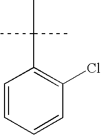 | (CD₃OD): 8.29(s, 1H), 7.86(s, 1H), 7.73(s, 1H), 7.71(d, J=8.4Hz, 1H), 7.48(d, J=7.6Hz, 1H), 7.40(d, J=8.4Hz, 1H), 7.33(t, J=7.6Hz, 1H), 7.19(t, J= |
| #7.6Hz, 1H), 7.09(d, J=7.6Hz, 1H), 4.36 (t, J=6.8Hz, 2H), 3.60(bs, 4H), 2.32(bs, 4H), 2.26(t, J=6.8Hz, 2H), 2.04(t, J=6.8Hz, 2H) | | | | |
| 25 | (5Z)-2-[(2,6-dichlorophenyl)- amino]-5-({1-[3-(4- (morpholinyl)propyl]-1H- benzimidazol-6-yl}- methylidene)- 1,3-thiazol-4(5H)-one | 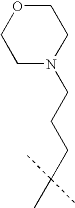 | 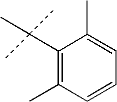 | (CD₃OD): 9.09(m, 1H), 8.01(bs, 1H), 7.91(s, 1H), 7.85(m, 1H), 7.61(m, 1H), 7.45(d, J=8.0Hz, 2H), 7.16(t, J=8.0Hz, 1H), 4.58 (bs, 2H), 4.02(m, 2H), 3.75(m, |
| #2H), 3.47(m, 2H), 3.25(bs, 2H), 3.12(m, 2H), 2.42(bs, 2H) | | | | |
| 26 | (5Z)-2-[(2,-chlorophenyl)- amino]-5-({1-[3-(4- methyl-1-piperazinyl)- propyl]-1H- benzimidazol-6-yl}- methylidene)- 1,3-thiazol-4(5H)-one | 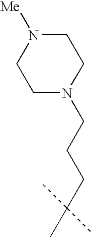 | 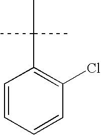 | (CD₃OD): 8.27(s, 1H), 7.81(s, 1H), 7.74(s, 1H), 7.70(d, J=8.8Hz, 1H), 7.47(d, J=8.0Hz, 1H), 7.39(d, J=8.4Hz, 1H), 7.32(t, J= |
| # 7.2Hz, 1H), 7.18(t, J=7.2Hz, 1H), 7.09(d, J=7.6Hz, 1H), 4.34 (t, J=6.4Hz, 2H), 2.46(bs, 8H), 2.30(s, 3H), 2.27(t, J=6.4Hz, 2H), 2.03(t, J=6.4Hz, 2H) | | | | |
| 27 | (5Z)-2-[(2,6-dichlorophenyl)- amino]-5-({1-[3-(4- methyl-1-piperazinyl)- propyl]-1H- benzimidazol-6-yl}- methylidene)- 1,3-thiazol-4(5H)-one | 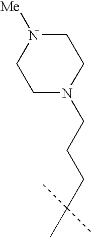 | 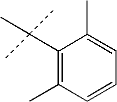 | (CD₃OD): 9.37(s, 1H), 8.09(s, 1H), 7.93(s, 1H), 7.89(d, J=8.4Hz, 1H), 7.69(d, J=8.0Hz, 1H), 7.35(d, J=8.4Hz, 2H), 7.16(t, J= |
| #8.0Hz, 1H), 4.59(m, 2H), 2.89 (s, 3H), 2.83(m, 8H), 2.63(bs, 2H), 2.22(t, J=6.4Hz, 2H) | | | | |
| 28 | (5Z)-2-[(2-chlorophenyl)- amino]-5-({1-[2-(1- pyrrolidinyl)- ethyl]-1H- benzimidazol-6-yl}- methylidene)- 1,3-thiazol-4(5H)-one | 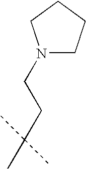 | 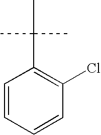 | (CD₃OD): 8.29(s, 1H), 7.84(s, 1H), 7.71(m, 2H), 7.47(dd, J=0.8, 8.0Hz, 1H), 7.41(d, J=8.0Hz, 1H), 7.32(dt, J=0.8, 7.6Hz, 1H), 7.18(dt, J= |
| #0.8, 7.6Hz, 1H), 7.09(dd, J=1.6, 8.0Hz, 1H), 4.40 (t, J=6.4Hz, 2H), 2.93(t, J=6.8Hz, 2H), 2.53(bs, 4H), 1.76(bs, 4H) | | | | |
| 29 | (5Z)-2-[(2,6-dichlorophenyl)- amino]-5-({1-[2-(1- pyrrolidinyl)- ethyl]-1H- benzimidazol-6-yl}- methylidene)- 1,3-thiazol-4(5H)-one | 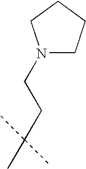 | 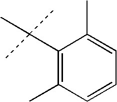 | (CD₃OD): 8.30(s, 1H), 7.87(s, 1H), 7.72(m, 2H), 7.44(d, J=8.0Hz, 2H), 7.40(dd, J=0.4, 9.2Hz, 1H), 7.14(t, J=8.0Hz, 1H), 4.43 |
| #(t, J=6.4Hz, 2H), 2.96(m, 2H), 2.56(bs, 4H), 1.78(bs, 4H) | | | | |
| 30 | (5Z)-2-[(2-chlorophenyl)- amino]-5-({1-[2-(1- piperidinyl)- ethyl]-1H- benzimidazol-6-yl}- methylidene)- 1,3-thiazol-4(5H)-one | 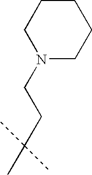 | 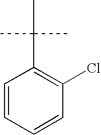 | (CD₃OD): 8.29(s, 1H), 7.89(s, 1H), 7.85(m, 2H), 7.47(d, J=6.8Hz, 1H), 7.41(d, J=7.2Hz, 1H), |
| #7.33(t, J=8.0Hz, 1H), 7.19(t, J=7.6Hz, 1H), 7.09(d, J=8.0Hz, 1H), 4.40(2H, 6.4Hz, 2H), 2.74(t, J=6.4Hz, 2H), 2.44(bs, 4H), 1.54(m, 6H) | | | | |
| 31 | (5Z)-2-[(2,6-dichlorophenyl)- amino]-5-({1-[2-(1- piperidinyl)- ethyl]-1H- benzimidazol-6-yl}- methylidene)- 1,3-thiazol-4(5H)-one | 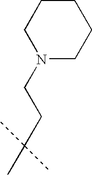 | 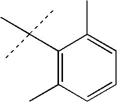 | (CD₃OD): 8.29(s, 1H), 7.86(s, 1H), 7.71(m, 2H), 7.44(d, J=8.0Hz, 2H), 7.41(d, J=8.8Hz, 1H), 7.14(t, J=8.0Hz, 1H), 4.40(t, J=6.0Hz, 2H), 2.73(t, J= |
| #6.0Hz, 2H), 2.44(bs, 4H), 1.54(m, 6H) | | | | |
| 32 | (5Z)-2-[(2,6-difluorophenyl)- amino]-5-({1-[2-(1- piperidinyl)- ethyl]-1H- benzimidazol-6-yl}- methylidene)- 1,3-thiazol-4(5H)-one | 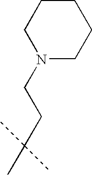 | 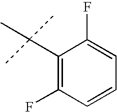 | (CD₃OD): 8.32(s, 1H), 7.84(s, 1H), 7.80(s, 1H), 7.70(d, J=8.8Hz, 1H), 7.38(d, J=8.4Hz, 1H), 7.21(m, 1H), 7.05(t, J=8.0Hz, 2H), 4.50(t, J=6.8Hz, 2H), 2.90 |
| #(t, J=6.8Hz, 2H), 2.60(bs, 4H), 1.62(m, 4H), 1.49(m, 2H) | | | | |

### Example 33

### (5Z)-2-[(2-Chlorophenyl)amino]-5-({1-[2-(dimethylamino)ethyl]-2-methyl-1-benzimidazol-6-yl}methylidene)-1,3-thiazol-4(5H)-one

(a) 4-Amino-3-{[2-(dimethylamino)ethyl]amino}benzonitrile. Following the procedure outlined in Example 15(a), 3-methoxy-4-nitrobenzonitrile was converted into 4-amino-3-{[2-(dimethylamino)ethyl]amino}benzonitrile in quantitative yield using N,N-dimethylethylenediamine as the nucleophile. ¹H NMR (CDCl₃): δ 7.01 (dd, J=1.6, 8.0 Hz, 1H), 6.80 (d, J=1.6 Hz, 1H), 6.65 (d, J=8.0 Hz, 1H), 3.98 (m, 2H), 3.12 (m, 2H), 2.62 (t, J=6.0 Hz, 2H), 2.26 (s, 3H).

(b) 1-[2-(Dimethylamino)ethyl]-2-methyl-1H-benzimidazole-6-carbaldehyde. The title compound was synthesized according to the procedure in Example 9(a) and 9(b), starting from 4-amino-3-{[2-(dimethylamino)ethyl]amino}benzonitrile. ¹H NMR (CDCl₃): δ 10.07 (s, 1H), 7.88 (d, J=1.2 Hz, 1H), 7.77 (s, 2H0, 4.25 (m, 2H), 2.68 (s, 3H), 2.67 (m, 2H), 2.31 (s, 6H).

(c)(5Z)-2-[(2-Chlorophenyl)amino]-5-({1-[2-(dimethylamino)ethyl]-2-methyl-1H-benzimidazol-6-yl}methylidene)-1,3-thiazol-4(5H)-one. Knoevenagel coupling with 2-[(2-chlorophenyl)amino]-1,3-thiazol-4(5H)-one was accomplished using the procedure of Example 1(e).

| Example | Compound name | NMR(400MHz) |
|---|---|---|
| 33 | (5Z)-2-[(2-Chlorophenyl)amino]- | (CD₃OD): 7.81(s, 1H), 7.60(m, 2H), 7.47(d, J=8.0Hz, |
| | 5-({1-[2-(dimethylamino)ethyl]- | 1H), 7.36(d, J=8.0Hz, 1H), 7.32(t, J=7.6Hz, |
| | 2-methyl-1H-benzimidazol- | 1H), 7.18(t, J=7.6Hz, 1H), 7.08(d, J=7.6Hz, |
| | 6-yl}methylidene)-1,3-thiazol- | 1H), 4.28(t, J=7.2Hz, 2H), 2.64(t, J=7.2Hz, |
| | 4(5H)-one | 2H), 2.61(s, 3H), 2.21(s, 6H) |

### Examples 34-40

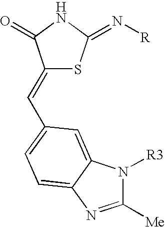

The following compounds were prepared according to the procedure of Example 33, using the requisite amine for N,N-dimethylethylenediamine and substituting the appropriate thiazolone for 2-[(2-chlorophenyl)amino]-1,3-thiazol-4(5H)-one:

| Example | Compound name | R3 | R | NMR (400 MHz) |
|---|---|---|---|---|
| 34 | (5Z)-2-[(2,6-dichlorophenyl)- amino]-5-({1-[2- (dimethylamino)ethyl]-2- methyl-1H-benzimidazol-6-yl}- methylidene-1,3- thiazol-4(5H)-one | 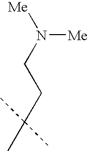 | 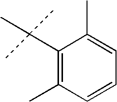 | (CD₃OD): 7.84(s, 1H), 7.58(m, 2H), 7.44(d, J=8.0Hz, 2H), 7.36(d, J=8.8Hz, 1H), 7.15(t, |
| #J=8.0Hz, 1H), 4.28(t, J=6.8Hz, 2H), 2.64(t, J=6.8Hz, 2H), 2.62(s, 3H), 2.21(s, 6H) | | | | |
| 35 | (5Z)-2-[(2,4-difluorophenyl)- amino]-5-({1-[2- (dimethylamino)ethyl]-2- methyl-1H-benzimidazol-6-yl}- methylidene-1,3- thiazol-4(5H)-one | 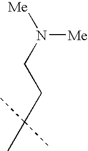 | 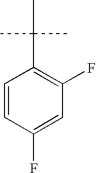 | (CD₃OD): 7.84(s, 1H), 7.61(m, 2H), 7.38(dd, J=1.2, 8.0Hz, 1H), 6.99-7.14(m, 3H), 4.34(t, J=7.6Hz, 2H), 2.74(t, J=7.6Hz, 2H), 2.63(s, 3H), 2.31(s, 6H) |
| 36 | (5Z)-5-({1-[2-(dimethylamino)- ethyl]-2-methyl-1H- benzimidazol-6-yl}- methylidene)-2-(phenylamino)- 1,3-thiazol-4(5H)-one | 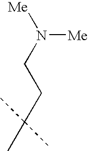 | 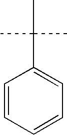 | (CD₃OD, mixture of two isomers): 7.09-7.95(m, 9H), 4.28-4.39(m, 2H), 2.61-2.76(m, 5H), 2.37(s, 3H), 2.23(s, 3H) |
| 37 | (5Z)-2-[(2,6-dichlorophenyl)- amino]-5-{[1-(2- hydroxyethyl)-2- methyl-1H-benzimidazol-6-yl]- methylidene}-1,3- thiazol-4(5H)-one | 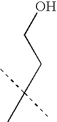 | 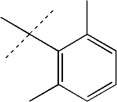 | (CD₃OD): 7.85(s, 1H), 7.63(s, 1H), 7.58(d, J=7.6Hz, 1H), 7.35(d, J=8.0Hz, 2H), 7.32(d, J=8.0Hz, 1H), 7.15(t, J=8.0Hz, 1H), 4.32(t, J=4.4Hz, |
| #2H), 3.85(t, J=4.4Hz, 2H), 2.64(s, 3H) | | | | |
| 38 | (5Z)-2-[(2-chlorophenyl)- amino]-5-({2-methyl-1- [2-(1-piperidinyl)ethyl]- 1H-benzimidazol-6-yl}- methylidene)-1,3- thiazol-4(5H)-one | 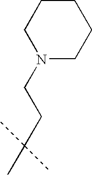 | 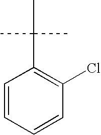 | (CD₃OD): 7.83(s, 1H), 7.60(m, 2H), 7.48(d, J=8.0Hz, 1H), 7.44(m, 2H), 7.18(t, J=8.0Hz, 1H), 7.09(d, J=7.2Hz, 1H), 4.31(t, J=6.4Hz, 2H), 2.65(t, |
| #J=6.4Hz, 2H), 2.63(s, 3H), 2.43(bs, 4H), 1.54(m, 4H), 1.46 (m, 2H) | | | | |
| 39 | (5Z)-2-[(2,6-dichlorophenyl)- amino]-5-({2- methyl-1-[2-(1-piperidinyl)ethyl]- 1H-benzimidazol-6-yl}- methylidene)-1,3-thiazol-4(5H)-one | 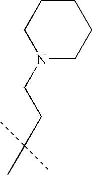 | 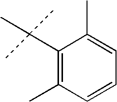 | (CD₃OD): 7.85(s, 1H), 7.68(m, 2H), 7.44(d, J=8.4Hz, 2H), 7.34(dd, J=1.2, 8.0Hz, 1H), 7.14(t, J=8.0Hz, 1H), 4.31(t, J=6.4Hz, 2H), 2.65(t, J= |
| #6.0Hz, 2H), 2.63(s, 3H), 2.44(bs, 4H), 1.54(m, 4H), 1.46(m, 2H) | | | | |
| 40 | (5Z)-2-[(2,6-difluorophenyl)- amino]-5-({2- methyl-1-[2-(1-piperidinyl)ethyl]- 1H-benzimidazol-6-yl}- methylidene)-1,3- thiazol-4(5H)-one | 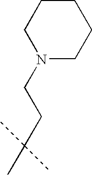 | 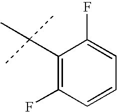 | (CD₃OD): 7.85(s, 1H), 7.59(m, 2H), 7.35(dd, J=1.2, 8.4Hz, 1H), 7.19(m, 1H), 7.05(t, J=8.0Hz, 2H), 4.33(t, J= |
| #6.8Hz, 2H), 2.69(t, J=6.8Hz, 2H), 2.63(s, 3H), 2.47(bs, 1H), 1.55 (m, 4H), 1.47(m, 2H) | | | | |

### Example 41

### (5Z)-2-[(2-Chlorophenyl)amino]-5-[(1-methyl-2-{[2-(4-morpholinyl)ethyl]amino}-1H-benzimidazol-6-yl)methylidene]-1,3-thiazol-4(5H)-one

(a) 3-Methyl-2-oxo-2,3-dihydro-1H-benzimidazole-5-carbonitrile. A suspension of 4-amino-3-(methylamino)benzonitrile (0.500 g, 3.40 mmol) and 1,1′-carbonyldiimidazole (1.10 g, 6.80 mmol) in THF (8.5 mL) was stirred at room temperature for 2 days. The reaction mixture was filtered, the collected solid rinsed with small amounts of H₂O and EtOAc, and dried to afford the cyclic urea (0.489 g, 83%) as a pink solid. Additional material could be recovered from the filtrate by separating the organic layer, washing with 0.1N HCl (1×30 mL), brine (1×30 mL), and drying over Na₂SO₄. After filtration, removal of solvent yielded 40 mg (7%) of additional product. ¹H NMR (d₆-acetone): δ 10.23 (bs, 1H), 7.46 (s, 1H), 7.42 (dd, J=1.6, 8.0 Hz, 1H), 7.20 (d, J=8.0 Hz, 1H), 3.40 (s, 3H).

(b) 2-Chloro-1-methyl-1H-benzimidazole-6-carbonitrile. A mixture of 3-methyl-2-oxo-2,3-dihydro-1H-benzimidazole-5-carbonitrile (0.219 g, 1.26 mmol) in POCl₃ (2.5 mL) was heated to 105° C. overnight. Upon cooling, the excess POCl₃ was removed under reduced vacuum, the residue diluted with H₂O and CH₂Cl₂, and the solution made basic with 1N NaOH. The layers were separated and the aqueous layer further extracted with CH₂Cl₂ (2×30 mL). The combined organic layers were dried (Na₂SO₄), filtered, and concentrated to afford the 2-chlorobenzimidazole (0.191 g, 80%) as atan solid. ¹H NMR (CDCl₃): δ 7.76 (dd, J=0.8, 8.0 Hz, 1H), 7.65 (dd, J=0.8, 1.6 Hz, 1H), 7.55 (dd, J=1.6, 8.0 Hz, 1H), 3.85 (s, 3H).

(c) 1-Methyl-2-{[2-(4-morpholinyl)ethyl]amino}-1H-benzimidazole-6-carbonitrile. A solution of 2-chloro-1-methyl-1H-benzimidazole-6-carbonitrile (50 mg, 0.262 mmol) and 4-(2-aminoethyl)morpholine (136 mg, 1.05 mmol) in EtOH (1.3 mL) was heated in a sealed vial to 80° C. for 24 h. The cooled reaction mixture was concentrated and the crude residue purified by column chromatography to yield the 2-aminobenzimidazole (66 mg, 88%). ¹HNMR (CDCl₃): δ 7.45 (d, J=8.4 Hz, 1H), 7.39 (dd, J=1.2, 8.4 Hz, 1H), 7.32 (d, J=0.8 Hz, 1H), 5.25 (m, 1H), 3.74 (t, J=4.8 Hz, 4H), 3.64 (m, 2H), 3.53 (s, 3H), 2.70 (t, J=5.6 Hz, 2H), 2.53 (t, J=4.4 Hz, 4H).

(d) 1-Methyl-2-{[2-(4-morpholinyl)ethyl]amino}-1H-benzimidazole-6-carbaldehyde. Following the procedure of Example 9(b), the benzimidazole-6-carbonitrile from Example 41(c) was reduced to the aldehyde in 73% yield. ¹H NMR (CDCl₃): δ 9.56 (s, 1H), 7.63 (m, 2H), 7.50 (d, J=8.0 Hz, 1H), 5.27 (bs, 1H), 3.74 (t, J=4.8 Hz, 4H), 3.66 (app. quartet, J=4.8, 11.2 Hz, 2H), 3.56 (s, 3H), 2.71 (t, J=5.6 Hz, 2H), 2.54 (t, J=4.4 Hz, 4H).

(e)(5Z)-2-[(2-Chlorophenyl)amino]-5-[(1-methyl-2-{[2-(4-morpholinyl)ethyl]amino-1H-benzimidazol-6-yl)methylidene]-1,3-thiazol-4(5H)-one. Knoevenagel coupling with 2-[(2-chlorophenyl)amino]-1,3-thiazol-4(5H)-one was accomplished using the procedure of Example 1(e).

| Example | Compound name | NMR(400MHz) |
|---|---|---|
| 41 | (5Z)-2-[(2-Chlorophenyl)- | (d₆-acetone): 7.76(s, 1H), |
| | amino]-5-[(1-methyl- | 7.51(m, 1H), 7.37(t, J=7.2Hz, |
| | 2-{[2-(4- | 1H), 7.30(m, 2H), 7.21(m, 2H), |
| | morpholinyl)ethyl]- | 7.16(m, 1H), 3.60(m, 10H), |
| | amino}-1H- | 2.64(m, 2H), 2.49(bs, 4H) |
| | benzimidazol- | |
| | 6-yl)methylidene]-1,3- | |
| | thiazol-4(5H)-one | |

Examples 42-45

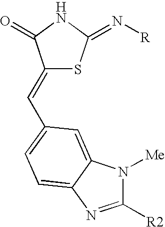

The following compounds were prepared according to the procedure of Example 41, using the requisite amine for 4-(2-aminoethyl)morpholine and substituting the appropriate thiazolone for 2-[(2-chlorophenyl)amino]-1,3-thiazol-4(5H)-one:

| Example | Compound name | R2 | R | NMR (400 MHz) |
|---|---|---|---|---|
| 42 | (5Z)-2-[(2,6-dichlorophenyl)- amino]-5-[(1-methyl-2- {[2-(4-morpholinyl)ethyl]- amino}-1H-benzimidazol-6-yl)- methylidene]-1,3- thiazol-4(5H)-one | 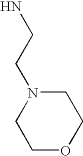 | 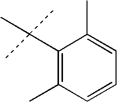 | (CD₃OD): 7.67(s, 1H), 7.41(d, J=8.4Hz, 2H), 7.28(m, 1H), 7.19 (m, 2H), 7.11(m, 1H), 3.71(m, 4H), 3.59(m, 2H), 3.50(s, 3H), 3.08(t, J= |
| #7.2Hz, 2H), 2.68(m, 2H), 2.55(m, 4H) | | | | |
| 43 | (5Z)-2-[(2,4-difluorophenyl)- amino]-5-[(1-methyl-2- {[2-(4-morpholinyl)ethyl]- amino}-1H-benzimidazol-6-yl)- methylidene]-1,3- thiazol-4(5H)-one | 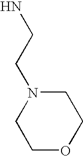 | 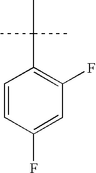 | (CD₃OD): 7.74(s, 1H), 7.37(m, 1H), 7.29(m, 1H), 7.22(m, 1H), 7.06(m, 3H), 3.70(bs, 4H), 3.59 (m, 2H), 3.52(s, 3H), 2.68(m, 2H), 2.56(bs, 4H). |
| 44 | (5Z)-2-[(2-chlorophenyl)- amino]-5-[(2-{[2-(dimethyl- amino)ethyl]amino}-1-methyl- 1H-benzimidazol-6-yl)- methylidene]-1,3- thiazol-4(5H)-one | 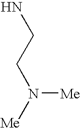 | 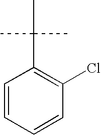 | (CD₃OD): 7.71(s, 1H), 7.47(d, J=7.6Hz, 1H), 7.32(m, 2H), 7.17 (m, 3H), 7.10(m, 1H), 3.61(t, J=6.0Hz, 2H), 3.51(s, 3H), 2.73(t, J=6.4Hz, 2H), 2.39(s, 6H) |
| 45 | (5Z)-2-[(2-chlorophenyl)- amino]-5-({2-[(2-hydroxyethyl)- amino]-1-methyl- 1H-benzimidazol-6-yl}- methylidene)-1,3- thiazol-4(5H)-one | 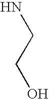 | 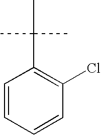 | (CD₃OD): 7.76(s, 1H), 7.48(d, J=8.0Hz, 1H), 7.33(t, J=7.6Hz, 1H), 7.28(dd, J=0.8, 7.2Hz, 1H), 7.20(m, 3H), 7.09(m, 1H), 3.76 (t, J=5.2Hz, 2H), 3.56(t, J= |
| #5.2Hz, 2H), 3.51(s, 3H) | | | | |

### Example 46

### (5Z)-2-[(2-Chlorophenyl)amino]-5-{[1-methyl-2-(4-morpholinylmethyl)-1H-benzimidazol-6-yl]methylidene}-1,3-thiazol-4(5H)-one

(a) 2-(Chloromethyl)-1-methyl-1H-benzimidazole-6-carbonitrile. To a mixture of 4-amino-3-(methylamino)benzonitrile (0.20 g, 1.36 mmol) in EtOH (6.8 mL) was added ethyl 2-chloroethanimidoate hydrochloride (0.43 g, 2.72 mmol; prepared according to Stillings et al. in J. Med. Chem. 1986, 29, 2280-2284). The mixture was stirred overnight and the solvent removed. The residue was diluted with H₂O and extracted with CH₂Cl₂ (3×20 mL). The combined organic layers were dried (Na₂SO₄), filtered and concentrated under reduced vacuum to afford the crude benzimidazole (0.266 g, 95%). ¹H NMR (CDCl₃): δ 7.82 (d, J=8.4 Hz, 1H), 7.72 (s, 1H), 7.56 (dd, J=1.2, 8.4 Hz, 1H), 4.86 (s, 2H), 3.93 (s, 3H).

(b) 1-Methyl-2-(4-morpholinylmethyl)-1H-benzimidazole-6-carbonitrile. To a mixture of 2-(chloromethyl)-1-methyl-1H-benzimidazole-6-carbonitrile (60 mg, 0.293 mmol) in EtOH (1 mL) was added morpholine (0.1 mL, 1.17 mmol). The mixture was heated to reflux for 1 h, cooled and concentrated to dryness. The residue was taken up in CH₂Cl₂ and treated with sat. aq. NaHCO₃. The layers were separated and the aqueous layer further extracted with CH₂Cl₂ (2×). The combined organic layers were dried over Na₂SO₄, filtered and the solvent removed in vacuo to provide the 2-methylmorpholino benzimidazole (73 mg, 100%). ¹H NMR (CDCl₃): δ 7.78 (d, J=8.4 Hz, 1H), 7.69 (s, 1H), 7.52 (dd, J=1.2, 8.4 Hz, 1H), 3.93 (s, 3H), 3.85 (s, 2H), 3.70 (t, J=4.8 H, 4H), 2.54 (t, J=4.8 Hz, 4H).

(c) 1-Methyl-2-(4-morpholinylmethyl)-1H-benzimidazole-6-carbaldehyde. Following the procedure of Example 9(b), the benzimidazole-6-carbonitrile from Example 46(b) was reduced to the aldehyde in 100% yield. ¹H NMR (CDCl₃): δ 10.10 (s, 1H), 7.95 (s, 1H), 7.83 (d, J=8.0 Hz, 1H), 7.79 (dd, J=1.2, 8.0 Hz, 1H), 3.97 (s, 3H), 3.86 (s, 2H), 3.71 (t, J=4.8 Hz, 4H), 2.55 (t, J=4.8 Hz, 4H).

(d) (5Z)-2-[(2-Chlorophenyl)amino]-5-{[1-methyl-2-(4-morpholinylmethyl)-1H-benzimidazol-6-yl]methylidene}-1,3-thiazol-4(5H)-one. Knoevenagel coupling with 2-[(2-chlorophenyl)amino]-1,3-thiazol-4(5H)-one was accomplished using the procedure of Example 1(e).

| Example | Compound name | NMR(400MHz) |
|---|---|---|
| 46 | (5Z)-2-[(2-Chlorophenyl)- | (CD₃OD): 7.81(s, 1H), 7.62(m, 2H), |
| | amino]-5-[(1-methyl- | 7.47(dd, J=1.2, 8.0Hz, 1H), 7.38(d, J=8.4Hz, |
| | 2-{[2-(4-morpholinyl)ethyl]- | 1H), 7.32(dt, J=1.2, 7.6Hz, 1H), |
| | amino}-1H-benzimidazol- | 7.18(dt, J=1.2, 8.0Hz, 1H), 7.09(dd, J=0.8, |
| | 6-yl)methylidene]-1,3- | 7.6Hz, 1H), 3.91(s, 3H), 3.83(s, 2H), |
| | thiazol-4(5H)-one | 3.66(bs, 4H), 2.50(bs, 4H) |

### Examples 47-49

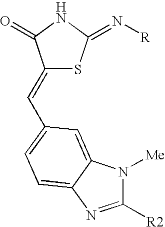

The following compounds were prepared according to the procedure of Example 46, using the requisite amine nucleophile and substituting the appropriate thiazolone for 2-[(2-chlorophenyl)amino]-1,3-thiazol-4(5H)-one:

| Example | Compound name | R2 | R | NMR (400 MHz) |
|---|---|---|---|---|
| 47 | (5Z)-2-[(2,6-dichlorophenyl)- amino]-5-{[1-methyl-2- (4-morpholinylmethyl)- 1H-benzimidazol-6-yl]- methylidene}-1,3- thiazol-4(5H)-one | 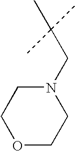 | 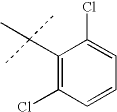 | (CD₃OD): 7.68(m, 1H), 7.60 (m, 2H), 7.40(m, 3H), 7.09 (m, 1H), 3.90(s, 3H), 3.82(s, 2H), 3.67(bs, 4H), 2.50(bs, 4H) |
| 48 | (5Z)-2-[(2-chlorophenyl)- amino]-5-({1-methyl-2- [(4-methyl-1-piperazinyl)methyl]- 1H-benzimidazol-6-yl}- methylidene)-1,3- thiazol-4(5H)-one | 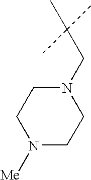 | 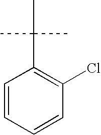 | (CD₃OD): 7.90(m, 1H), 7.87 (s, 1H), 7.80(m, 1H), 7.64(m, 1H), 7.47(d, J=7.6Hz, 1H), 7.30(t, J=6.8Hz, 1H), 7.19 (t, J= |
| #7.2Hz, 1H), 7.08(m, 1H), 4.17(m, 2H), 3.99(s, 3H), 3.50(m, 2H), 3.12(m, 4H), 2.92(s, 3H), 2.70(m, 2H) | | | | |
| 49 | (5Z)-2-[(2,6-dichlorophenyl)- amino]-5-({1-methyl-2- [(4-methyl-1-piperazinyl)- methyl]-1H-benzimidazol-6-yl}- methylidene)-1,3- thiazol-4(5H)-one | 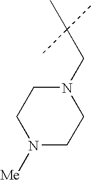 | 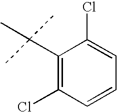 | (CD₃OD): 7.90(s, 1H), 7.89 (s, 1H), 7.78(d, J=8.4Hz, 1H), 7.57(t, J=7.6Hz, 1H), 7.44(d, J=8.0Hz, 2H), 7.15 (t, J= |
| #8.4Hz, 1H), 4.14(s, 2H), 3.98(s, 3H), 3.50(m, 2H), 3.12(m, 4H), 2.91(s, 3H), 2.68(m, 2H) | | | | |

### Example 50

### (5Z)-2-[(2-Chlorophenyl)amino]-5-{[1-methyl-2-(trifluoromethyl)-1H-benzimidazol-6-yl]methylidene}-1,3-thiazol-4(5H)-one

(a) 1-Methyl-2-(trifluoromethyl)-1H-benzimidazole-6-carbonitrile. 4-Amino-3-(methylamino)benzonitrile (80 mg, 0.544 mmol) and trifluoroacetic acid (1.1 mL) were heated to reflux for 6 h. Upon cooling, the excess TFA was removed under reduced pressure and sat. aq. NaHCO₃ carefully added. A beige solid precipitated, which was filtered, rinsed with H₂O and dried to afford the 2-trifluoromethyl benzimidazole (100 mg, 82%). ¹H NMR (CDCl₃): δ 7.98 (d, J=8.0 Hz, 1H), 7.84 (s, 1H), 7.65 (dd, J=1.2, 8.0 Hz, 1H), 4.01 (s, 3H).

(b) 1-Methyl-2-(trifluoromethyl)-1H-benzimidazole-6-carbaldehyde. Following the procedure of Example 9(b), the benzimidazole-6-carbonitrile from Example 50(a) was reduced to the aldehyde in 70% yield. ¹H NMR (CDCl₃): δ 10.15 (s, 1H), 8.06 (s, 1H), 8.01 (d, J=8.4 Hz, 1H), 7.92 (d, J=8.0 Hz, 1H), 4.04 (s, 3H).

(c)(5Z)-2-[(2-Chlorophenyl)amino]-5-{[1-methyl-2-(trifluoromethyl)-1H-benzimidazol-6-yl]methylidene}-1,3-thiazol-4(5H)-one. Knoevenagel coupling with 2-[(2-chlorophenyl)amino]-1,3-thiazol-4(5H)-one was accomplished using the procedure of Example 1(e).

| Example | Compound name | NMR(400MHz) |
|---|---|---|
| 50 | (5Z)-2-[(2-Chlorophenyl)- | (CD₃OD): 7.88(s, 1H), 7.82(m, |
| | amino]-5-{[1-methyl-2- | 2H), 7.52(d, J=8.8Hz, 1H), |
| | (trifluoromethyl)- | 7.48(d, J=8.0Hz, 1H), 7.33(t, |
| | 1H-benzimidazol-6-yl]- | J=7.6Hz, 1H), 7.19(t, J=7.6Hz, |
| | methylidene}-1,3- | 1H), 7.10(d, J=7.6Hz, 1H), |
| | thiazol-4(5H)-one | 3.99(s, 3H) |

### Examples 51-52

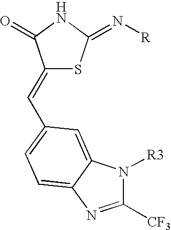

The following compounds were prepared according to the procedure of Example 50, using the requisite diaminobenzonitrile starting material and substituting the appropriate thiazolone for 2-[(2-chlorophenyl)amino]-1,3-thiazol-4(5H)-one:

| Example | Compound name | R3 | R | NMR (400 MHz) |
|---|---|---|---|---|
| 51 | (5Z)-2-[(2,6-dichlorophenyl)- amino]-5-{[1-methyl-2- (trifluoromethyl)-1H- benzimidazol-6-yl]- methylidene}-1,3- thiazol-4(5H)-one | Me | 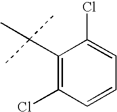 | (CD₃OD): 7.90(s, 1H), 7.81(m, 2H), 7.50(dd, J=0.8, 8.4Hz, 1H), 7.44(d, J=8.4Hz, 2H), 7.15(d, J=8.0Hz, 1H), 4.00(s, 3H) |
| 52 | (5Z)-2-[(2,6-dichlorophenyl)- amino]-5-{[1-[2- (dimethylamino)ethyl]- 2-(trifluoromethyl)-1H- benzimidazol-6-yl]- methylidene}-1,3- thiazol-4(5H)-one | 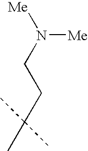 | 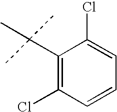 | (CD₃OD): 7.90(m, 3H), 7.55(d, J=8.0Hz, 1H), 7.46(d, J=8.0Hz, 2H), 7.17(t, J= |
| #8.0Hz, 1H), 3.59(m, 2H), 3.06(m, 2H), 3.02(s, 6H) | | | | |

### Example 53

### (5Z)-2-[(2-Chlorophenyl)amino]-5-{[2-(1,1-dimethylethyl)-1-methyl-1H-benzimidazol-6-yl]methylidene}-1,3-thiazol-4(5H)-one

(a) 2-(1,1-Dimethylethyl)-1-methyl-1H-benzimidazole-6-carbonitrile. To a mixture of Cu(OAc)₂ (800 mg, 4.76 mmol) in AcOH (3.6 mL) and H₂O (1.2 mL), heated to 55° C., was added 4-amino-3-(methylamino)benzonitrile (70 mg, 0.48 mmol) and pivaldehyde (57 μL, 0.52 mmol). Heating was continued for 2 h, then the solvent was removed under reduced pressure. EtOAc (50 ml) was added, which was washed with sat. aq. NaHCO₃. The layers were separated, the aqueous layer extracted with EtOAc (20 mL), and the combined organic layer washed with brine (2×20 mL), dried (Na₂SO₄), filtered and concentrated to afford the 2-t-butyl benzimidazole (86 mg, 85%). ¹H NMR (CDCl₃): δ 7.78 (d, J=8.4 Hz, 1H), 7.62 (d, J=1.2 Hz, 1H), 7.50 (dd, J=1.2, 8.4 Hz, 1H), 3.95 (s, 3H), 1.58 (s, 9H).

(b) 2-(1,1-Dimethylethyl)-1-methyl-1H-benzimidazole-6-carbaldehyde. Following the procedure of Example 9(b), the benzimidazole-6-carbonitrile from Example 53(a) was reduced to the aldehyde in 93% yield. ¹H NMR (CDCl₃): δ 10.08 (s, 1H), 7.89 (s, 1H), 7.84 (d, J=8.4 Hz, 1H), 7.77 (dd, J=1.2, 8.4 Hz, 1H), 3.99 (s, 3H), 1.59 (s, 9H).

(c)(5Z)-2-[(2-Chlorophenyl)amino]-5-{[2-(1,1-dimethylethyl)-1-methyl-1H-benzimidazol-6-yl]methylidene}-1,3-thiazol-4(5H)-one. Knoevenagel coupling with 2-[(2-chlorophenyl)amino]-1,3-thiazol-4(5H)-one was accomplished using the procedure of Example 1(e).

| Example | Compound name | NMR(400MHz) |
|---|---|---|
| 53 | (5Z)-2-[(2-chlorophenyl)amino]- | (CD₃OD): 7.97(s, 1H), 7.87(s, 1H), 7.80(d, J=8.8Hz, |
| | 5-{[2-(1,1-dimethylethyl)-1-methyl- | 1H), 7.70(d, J=8.4Hz, 1H), 7.47(d, J=8.0Hz, 1H), |
| | 1H-benzimidazol-6-yl]meth- | 7.32(dt, J=0.8, 7.6Hz, 1H), 7.19(dt, J=1.2, 8.0Hz, |
| | ylidene}-1,3-thiazol-4(5H)-one | 1H), 7.08(dd, J=1.2, 7.6Hz, 1H), 4.17(s, 3H), 1.66(s, |
| | | 9H) |

### Example 54

### (5Z)-2-[(2,6-Dichlorophenyl)amino]-5-{[2-(1,1-dimethylethyl)-1-methyl-1H-benzimidazol-6-yl]methylidene}-1,3-thiazol-4(5H)-one

The title compound was prepared according to the procedure of Example 53, except substituting 2-[(2,6-dichlorophenyl)amino]-1,3-thiazol-4(5H)-one for 2-[(2-chlorophenyl)amino]-1,3-thiazol-4(5H)-one:

| Example | Compound name | NMR(400MHz) |
|---|---|---|
| 54 | (5Z)-2-[(2,6-dichlorophenyl)ami- | (CD₃OD): 7.94(s, 1H), 7.91(s, 1H), 7.81(d, J=8.4Hz, |
| | no]-5-{[2-(1,1-dimethylethyl)-1- | 1H), 7.68(d, J=8.4Hz, 1H), 7.44(d, J=8.4Hz, 2H), |
| | methyl-1H-benzimidazol-6- | 7.15(t, J=8.4Hz, 1H), 4.18(s, 3H), 1.66(s, 9H) |
| | yl]ethylidene}-1,3-thiazol-4(5H)- | |
| | one | |

### Example 55

### (5Z)-2-[(2-Chlorophenyl)amino]-5-[(1-methyl-1H-1,2,3-benzotriazol-6-yl)methylidene]-1,3-thiazol-4(5H)-one

(a) 1-Methyl-1H-1,2,3-benzotriazole-6-carbonitrile. To a 0° C. mixture of 4-amino-3-(methylamino)benzonitrile (60 mg, 0.408 mmol) in conc. HCl (0.55 mL) was added NaNO₂ (31 mg, 0.449 mmol) in H₂O (0.2 mL). The mixture was allowed to warm to room temperature and stirred for 1 h. After recooling to 0° C., the mixture was treated with 6N NaOH until basic, the precipitate filtered, rinsed with H₂O and dried to afford the benzotriazole nitrile (50 mg, 77%). ¹H NMR (CDCl₃): δ 8.19 (d, J=8.4 Hz, 1H), 7.95 (s, 1H), 7.62 (d, J=8.4 Hz, 1H), 4.38 (s, 3H).

(b) 1-Methyl-1H-1,2,3-benzotriazole-6-carbaldehyde. Following the procedure of Example 9(b), the benzimidazole-6-carbonitrile from Example 55(a) was reduced to the aldehyde in 92% yield. ¹H NMR (CDCl₃): δ 10.20 (s, 1H), 8.20 (d, J=8.8 Hz, 1H), 8.11 (t, J=1.2 Hz, 1H), 7.93 (dd, J=1.2, 8.8 Hz, 1H), 4.41 (s, 3H).

(c)(5Z)-2-[(2-Chlorophenyl)amino]-5-[(1-methyl-1H-1,2,3-benzotriazol-6-yl)methylidene]-1,3-thiazol-4(5H)-one. Knoevenagel coupling with 2-[(2-chlorophenyl)amino]-1,3-thiazol-4(5H)-one was accomplished using the procedure of Example 1(e).

| Example | Compound name | NMR(400MHz) |
|---|---|---|
| 55 | (5Z)-2-[(2-Chlorophenyl) | (CD₃OD): 8.01(d, J=9.2Hz, |
| | amino]-5- | 1H), 7.87(s, 2H), 7.53(d, J=9.6Hz, |
| | [(1-methyl-1H-1,2,3- | 1H), 7.48(d, J=7.6Hz, |
| | benzotriazol-6- | 1H), 7.33(dt, J=0.8, 7.2Hz, |
| | yl)methylidene]- | 1H), 7.19(m, 1H), 7.09(d, |
| | 1,3-thiazol-4(5H)-one | J=8.0Hz, 1H), 4.32(s, 3H) |

### Examples 56-58

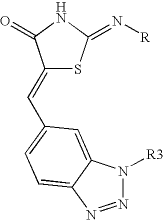

The following compounds were prepared according to the procedure of Example 55, using the requisite diaminobenzonitrile starting material and substituting the appropriate thiazolone for 2-[(2-chlorophenyl)amino]-1,3-thiazol-4(5H)-one:

| Example | Compound name | R3 | R | NMR (400 MHz) |
|---|---|---|---|---|
| 56 | (5Z)-2-[(2,6-dichlorophenyl)- amino]-5-[(1-methyl- 1H-1,2,3-benzotriazol-6-yl)- methylidene]-1,3- thiazol-4(5H)-one | Me | 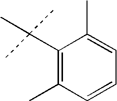 | (CD₃OD): 8.01(d, J=8.8Hz, 1H), 7.91(s, 1H), 7.87(s, 1H), 7.52(d, J=8.8Hz, 1H), 7.44(d, J=8.0Hz, 2H), 7.16(t, J=8.0Hz, 1H), 4.32(s, 3H) |
| 57 | (5Z)-2-[(2-chlorophenyl)- amino]-5-({1-[2-(dimethylamino)- ethyl]-1H-1,2,3-benzotriazol-6-yl}- methylidene)-1,3- thiazol-4(5H)-one | 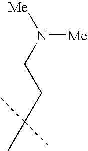 | 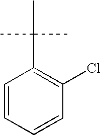 | (CD₃OD): 8.10(d, J=8.8Hz, 1H), 7.96(s, 1H), 7.89(s, 1H), 7.60(d, J=10.0Hz, 1H), 7.48(d, J=8.0Hz, 1H), 7.33(t, J=7.6Hz, 1H), 7.19(dt, J=1.2, 7.6Hz, |
| #1H), 7.08(d, J=8.0Hz, 1H), 5.15 (t, J=6.0Hz, 2H), 3.87(t, J=5.6Hz, 2H), 3.01(s, 6H) | | | | |
| 58 | (5Z)-2-[(2,6-dichlorophenyl)- amino]-5-({1-[2-(dimethylamino)- ethyl]-1H-1,2,3-benzotriazol-6-yl}- methylidene)-1,3- thiazol-4(5H)-one | 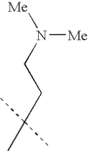 | 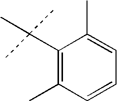 | (CD₃OD): 8.10(d, J=8.4Hz, 1H), 7.97(s, 1H), 7.91(s, 1H), 7.57(d, J=8.4Hz, 1H), 7.44(d, J=8.0Hz, 2H), 7.16(t, J=8.0Hz, 1H), 5.16(t, J= |
| #6.0Hz, 2H), 3.88 (t, J=6.0Hz, 2H), 3.01(s, 6H) | | | | |

### Example 59

### 2-(2,6-Dichloro-phenylimino)-5-(2-methyl-benzooxazol-6-yl-methylene)-thiazolidin-4-one

### a) 2-Methyl-benzooxazole-6-carboxylic acid methyl ester

A suspension of methyl 4-amino-3-hydroxy-benzoate (30 g, 0.18 mol) in triethylorthoacetate (90 mL) was heated to 100° C. for 3 hours. Ethanol (150 mL) was added followed by water (50 mL). The reaction mixture was filtered to yield 25 g (72% yield) of pure 2-methyl-benzooxazole-6-carboxylic acid methyl ester. ¹H-NMR (CDCl₃): δ 2.67 (s, 3H), 3.94 (s, 3H), 7.65 (d, 1H, J=8.1 Hz), 8.02 (dd, 1H, J=8.1 Hz, J′=1.5 Hz), 8.15 (d, 1H, J=1.5 Hz). LC MS (m/e)=192.2 (MH+). Rt=1.70 min

### b) (2-Methyl-benzooxazol-6-yl)-methanol

To the solution of 2-methyl-benzooxazole-6-carboxylic acid methyl ester (25 g, 0.13 mol) in THF (500 mL) at −20° C. was added a solution of lithium aluminum hydride (4.81 g, 130 mL of 1 M solution in THF, 0.13 mmol, 1 eq) and the reaction mixture was allowed to warm up to the room temperature overnight. Water (5 mL) followed by 1 M NaOH solution (5 mL) followed by water (15 mL) was added and the reaction mixture was stirred for 15 min at the room temperature. The suspension was filtered, liquid evaporated and purified by column chromatography (1:3 ethyl acetate:dichloromethane) to give 12.5 g (58% yield) of pure (2-methyl-benzooxazol-6-yl)-methanol. ¹H-NMR (CDCl₃): δ 2.64 (s, 3H), 4.82 (s, 2H), 7.29 (d, 1H, J=8 Hz), 7.53 (s, 1H), 7.62 (d, 1H, J=8 Hz). LC MS (m/e)=164.2 (MH+). Rt=1.03 min.

### c) 2-Methyl-benzooxazole-6-carbaldehyde

To the solution (2-methyl-benzooxazol-6-yl)-methanol (12.5 g, 76 mmol) in dichloromethane (200 mL) was added pyridinium chlorochromate (20 g, 93 mmol, 1.2 eq) and the reaction mixture was stirred for 1 hour at the room temperature. Celite (10 g) was added followed by decolorizing carbon (2 g) and the reaction mixture was filtered after 15 min of stirring. After evaporation the crude product was purified column chromatography (1:10 ethyl acetate:dichloromethane) of give 8.2 g (66% yield) of pure 2-methyl-benzooxazole-6-carbaldehyde. ¹H-NMR (CDCl₃): δ 2.73 (s, 3H), 7.79 (d, 1H, J=8.1 Hz), 7.88 (dd, 1H, J=8.1 Hz, J′=1.2 Hz), 8.03 (d, 1H, J=1.2 Hz), 10.09 (s, 1H). LC MS (m/e)=162.2 (MH+). Rt=1.47 min.

### d) 2-(2,6-Dichloro-phenylimino)-5-(2-methyl-benzooxazol-6-yl-methylene)-thiazolidin-4-one

To the solution of 2-(2,6 dichloro-phenylimino)-thiazolidin-4-one (483 mg, 1.85 mmol) in acetic acid (8 mL) was added 2-methyl-benzooxazole-6-carbaldehyde (300 mg, 1.85 mmol, 1 eq) followed by sodium acetate (0.8 g). The reaction mixture was refluxed for 48 hours and water (10 mL) was added. Solid was filtered and purified by column chromatography (1:5 ethyl acetate:dichloromethane) to give 110 mg (15% yield) of pure 2-(2,6-dichloro-phenylimino)-5-(2-methyl-benzooxazol-6-yl-methylene)-thiazolidin-4-one. ¹H-NMR (CDCl₃): δ 2.69 (s, 3H), 7.12 (t, 1H, J=8.1 Hz), 7.36 (d, 3H, J=7.8 Hz), 7.56 (s, 1H), 7.70 (d, 1H, J=8.1 Hz), 7.88 (s, 1H), 9.69 (s, 1H). LC MS (m/e)=404.0 (MH+). Rt=2.36 min.

### Example 60

### 2-(2,6-Difluoro-phenylimino)-5-(2-methyl-benzooxazol-6-yl-methylene)-thiazolidin-4-one

Following the procedure of example 59 (d), starting from 2-(2,6-difluoro-phenylimino)-thiazolidin-4-one, the title compound was prepared as a yellow solid (82 mg, 22%). ¹H-NMR (CDCl₃): δ 2.69 (s, 3H), 7.30 (t, 2H, J=7.9 Hz), 7.15 (m, 1H), 7.41 (d, 1H, J=8.3 Hz), 7.57 (s, 1H), 7.70 (d, 1H, J=8.1 Hz), 7.81 (s, 1H), LC MS (m/e)=372.0 (MH+). Rt=2.13 min.

### Example 61

### 2-(2-Fluoro-phenylimino)-5-(2-methyl-benzooxazol-6-yl-methylene)-thiazolidin-4-one

To the solution of 2-(2,6-difluoro-phenylimino)-thiazolidin-4-one (105 mg, 0.5 mmol) in ethanol (5 mL) was added 2-methyl-benzooxazole-6-carbaldehyde (80 mg, 0.5 mmol, 1 eq) followed by piperidine (0.1 mL). The reaction mixture was refluxed for 48 hours and diethyl ether (3 mL) was added. Solid was filtered to give 58 mg (33% yield) of pure 2-(2-fluoro-phenylimino)-5-(2-methyl-benzooxazol-6-yl-methylene)-thiazolidin-4-one. LC MS (m/e)=354.2 (MH+). Rt=2.11 min.

### Examples 62-71

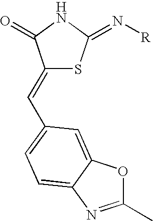

The following compounds were prepared according to the procedure of Example 61, except substituting the appropriately substituted thiazolidinone for 2-(2,6-difluoro-phenylimino)-thiazolidin-4-one.

| | | Thiazolidinone | LC MS | |
|---|---|---|---|---|
| Example | Product name | substituted | (m/e) | Rt (min) |
| 62 | 2-(2-Chloro-phenylimino)- 5-(2-methyl-benzooxazol- 6-ylmethylene)-thiazolidin- 4-one | 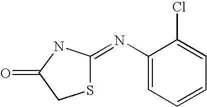 | 370.0 | 2.23 |
| 63 | 2-(2-Trifluromethyl- phenylimino)-5-(2-methyl- benzooxazol-6-ylmethylene)- thiazolidin-4-one | 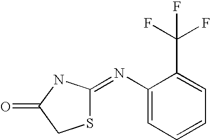 | 404.0 | 2.34 |
| 64 | 2-(2,4-Difluoro-phenylimino)- 5-(2-methyl-benzooxazol-6- ylmethylene)-thiazolidin- 4-one | 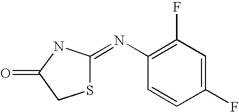 | 372.0 | 2.16 |
| 65 | 2-(2,5-Dichloro-phenylimino)- 5-(2-methyl-benzooxazol-6- ylmethylene)-thiazolidin- 4-one | 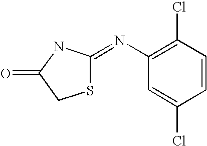 | 404.2 | 2.46 |
| 66 | 2-(2,4-Dimethyl-phenylimino)- 5-(2-methyl-benzooxazol-6- ylmethylene)-thiazolidin- 4-one | 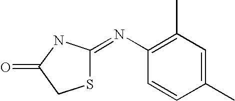 | 364.2 | 2.31 |
| 67 | 2-(4-Cyano-phenylimino)- 5-(2-methyl-benzooxazol-6- ylmethylene)-thiazolidin- 4-one | 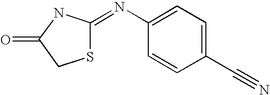 | 361.0 | 2.07 |
| 68 | 4-[5-(2-Methyl-benzooxazol-6- ylmethylene)-4-oxo-thiazolidin-2- ylideneamino]- benzoic acid | 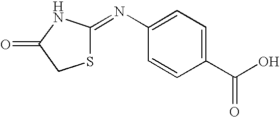 | 380.0 | 1.99 |
| 69 | 2-(2,4-Dichloro-phenylimino)- 5-(2-methyl-benzooxazol-6- ylmethylene)-thiazolidin- 4-one | 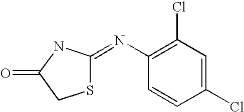 | 404.0 | 2.52 |
| 70 | 2-(2,5-Difluoro-phenylimino)- 5-(2-methyl-benzooxazol-6- ylmethylene)-thiazolidin- 4-one | 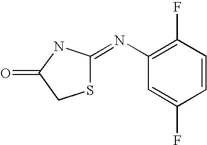 | 372.0 | 2.20 |
| 71 | 5-(2-Methyl-benzooxazol- 6-ylmethylene)-2-phenylimino- thiazolidin-4-one | 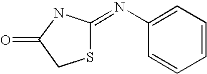 | 336.2 | 2.11 |

### Example 72

### 5-(2-Methyl-benzooxazol-6-ylmethylene)-2-(2-piperidin-1-yl-ethylimino)-thiazolidin-4-one

### a) 5-(2-Methyl-benzooxazol-6-ylmethylene)-2-thioxo-thiazolidin-4-one

To the solution of rhodanine (1.21 g, 10 mmol) in ethanol (50 mL) was added 2-methyl-benzooxazole-6-carbaldehyde (1.61 mg, 10 mmol, 1 eq) followed by pyridine (1 mL). The reaction mixture was refluxed for 24 hours cooled to the room temperature. Solid was filtered to give 1.3 g (47% yield) of pure 5-(2-methyl-benzooxazol-6-ylmethylene)-2-thioxo-thiazolidin-4-one. ¹H-NMR (DMSO): δ 2.67 (s, 3H), 2.85 (s, 3H), 7.66 (dd, 1H, J=8.3 Hz, J′=1.7 Hz), 7.82 (d, 1H, J=8.3 Hz), 8.00 (s, 1H), 8.02 (d, 1H, J=1.7 Hz). LC MS (m/e)=277.0 (MH+). Rt=2.02 min.

### b) 5-(2-Methyl-benzooxazol-6-ylmethylene)-2-methylsulfanyl-thiazol-4-one

To the solution of 5-(2-methyl-benzooxazol-6-ylmethylene)-2-thioxo-thiazolidin-4-one (200 mg, 0.72 mmol) in ethanol (5 mL) was added diisopropyl ethyl amine (0.185 mL, 1.44 mmol, 2 eq) followed by iodomethane (0.216 mL, 3.5 mmol, 5 eq). The reaction mixture was stirred overnight, then filtered. Solid was washed with cold ethanol to give 193 mg (92% yield) of pure 5-(2-methyl-benzooxazol-6-ylmethylene)-2-methylsulfanyl-thiazol-4-one. ¹H-NMR (DMSO): δ 2.67 (s, 3H), 7.59 (dd, 1H, J=8.3 Hz, J′=1.5 Hz), 7.80 (s, 1H), 7.82 (d, 1H, J=8.3 Hz), 7.96 (d, 1H, J=1.5 Hz). LC MS (m/e)=291.0 (MH+). Rt=2.41 min.

### c) 5-(2-Methyl-benzooxazol-6-ylmethylene)-2-(2-piperidin-1-yl-ethylimino)-thiazolidin-4-one

To the solution of 5-(2-methyl-benzooxazol-6-ylmethylene)-2-methylsulfanyl-thiazol-4-one (40 mg, 0.14 mmol) in ethanol (3 mL) was added 2-piperidin-1-yl-ethylamine (25 mg, 0.2 mmol, 1.4 eq) and the reaction mixture was heated under reflux for 24 hours. Diethyl ether (3 mL) was added and product isolated by filtration to give 27 mg (53% yield) of pure 5-(2-methyl-benzooxazol-6-ylmethylene)-2-(2-piperidin-1-yl-ethylimino)-thiazolidin-4-one. LC MS (m/e)=371.0 (MH+). Rt=1.40 min.

### Examples 73-85

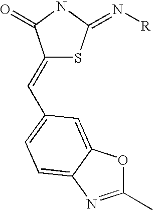

The following compounds were prepared according to the procedure of Example 72 (c), except substituting the appropriate amine listed below for 2-piperidin-1-yl-ethylamine.

| Example | Product name | Amine used | LC MS (m/e) | Rt (min) |
|---|---|---|---|---|
| 73 | 2-(2-Methoxy-ethylimino)-5- (2-methyl-benzooxazol-6- ylmethylene)-thiazolidin-4-one | 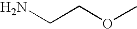 | 318.0 | 1.52 |
| 74 | 5-(2-Methyl-benzooxazol-6- ylmethylene)-2-(3-morpholin-4- yl-propylimino)-(thiazolidin- 4-one | 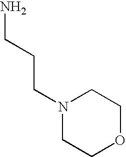 | 387.2 | 1.31 |
| 75 | 3-[5-(2-Methyl-benzooxazol-6- ylmethylene)-4-oxo-thiazolidin- 2-ylideneamino]- benzenesulfonamide | 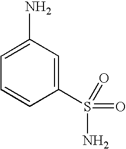 | 415.2 | 1.68 |
| 76 | 2-(4-Hydroxy-butylimino)-5- (2-methyl-benzooxazol-6- ylmethylene)-thiazolidin-4-one | 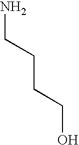 | 332.2 | 1.49 |
| 77 | 2-(trans-4-Hydroxy-cyclo- hexylimino)- 5-(2-methyl-benzooxazol-6- ylmethylene)-thiazolidin-4-one | 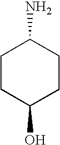 | 358.0 | 1.45 |
| 78 | 5-(2-Methyl-benzooxazol-6- ylmethylene)-2-phenethylimino- thiazolidin-4-one | 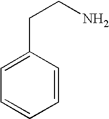 | 363.8 | 2.00 |
| 79 | 4-{2-[5-(2-Methyl-benzooxazol-6- ylmethylene)-4-oxo-thiazolidin- 2-ylideneamino]-ethyl}- benzenesulfonamide | 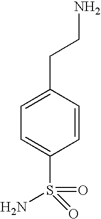 | 443.2 | 1.63 |
| 80 | 2-(2-Benzo[1,3]dioxol-5-yl- ethylimino)-5-(2-methyl- benzooxazol-6- ylmethylene)-thiazolidin- 4-one | 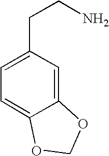 | 408.2 | 1.97 |
| 81 | 2-(4-Chloro-phenylimino)-5- (2-methyl-benzooxazol-6- ylmethylene)-thiazolidin-4-one | 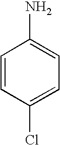 | 370.0 | 2.31 |
| 82 | 5-(2-Methyl-benzooxazol-6- ylmethylene)-2-(pyridin-3- ylimino)-thiazolidin-4-one | 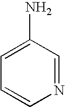 | 337.2 | 1.40 |
| 83 | 3-[5-(2-Methyl-benzooxazol-6- ylmethylene)-4-oxo-thiazolidin- 2-ylideneamino]- benzamide | 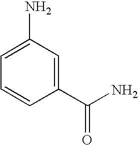 | 379.2 | 1.61 |
| 84 | 2-(2-Hydroxy-ethylimino)-5- (2-methyl-benzooxazol-6- ylmethylene)-thiazolidin-4-one | 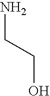 | 304.0 | 1.33 |
| 85 | 2-(1-Hydroxymethyl-2-phenyl- ethylimino)-5-(2-methyl- benzooxazol-6-ylmethylene)- thiazolidin-4-one | 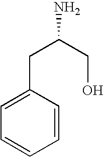 | 394.2 | 1.76 |

### Example 86

### N-{6-[2-(2-Bromo-phenylimino)-4-oxo-thiazolidin-5-ylidenemethyl]-1H-benzoimidazol-2-yl}-2-dimethylamino-acetamide

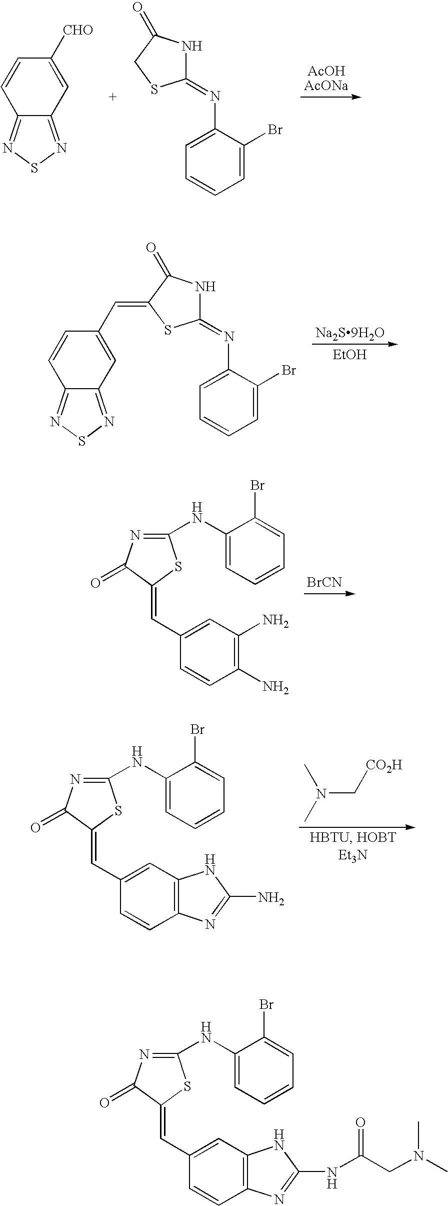

### a. 5-Benzo[1,2,5]thiadiazol-5-ylmethylene-2-(2-bromophenylimino)-thiazolidin-4-one

A mixture of benzo[1,2,5]thiadiazole-5-carbaldehyde (70 mg, 0.43 mmol), (2-bromophenylimino)-thiazolidin-4-one (110 mg, 0.40 mmol), AcONa (100 mg) in AcOH (2 mL) was heated to reflux at 120 degree for 48 hours. After cooling, a small portion of water was added until the solid forms. It was filtered and washed with MeOH, followed by desiccation in vacuo to afford a target product (104 mg, 0.25 mmol). ¹H NMR (DMSO-d₆) δ 7.15 (m, 2H), 7.43 (t, 1H), 7.71 (d, 1H), 7.83 (dd, 1H), 7.89 (s, 1H), 8.16 (d, 1H), 8.22 (s, 1H), 12.83 (sbr, 1H): LC/MS: m/z 417 (M), 419 (M+2)

### b. 2-(2-Bromo-phenylamino)-5-(3,4-diamino-benzylidene)-thiazol-4-one

A mixture 5-benzo[1,2,5]thiadiazol-5-ylmethylene-2-(2-bromophenylimino)-thiazolidin-4-one (380 mg) and Na₂S-9H₂O (600 mg) in ethanol was irradiated by a microwave reactor at 120° C. for 5 hours. The mixture was poured onto aq. NH₄Cl and the formed orange precipitate was filtrated. Washing with H₂O and subsequent desiccation gave 290 mg of the title product. ¹H NMR (DMSO-d₆) δ 4.68 (sbr, 2H), 5.30 (s, 2H), 6.44-6.55 (m, 3H), 7.04 (m, 2H), 7.29 (s, 1H), 7.33 (t, 1H), 7.61 (d, 1H): LC/MS: m/z 389 (M), 391 (M+2).

### c. 5-(2-Amino-3H-benzoimidazol-5-ylmethylene)-2-(2-bromo-phenylimino)-thiazolidin-4-one

A mixture of 2-(2-bromo-phenylamino)-5-(3,4-diamino-benzylidene)-thiazol-4-one (130 mg) and BrCN (120 mg) in methanol (1.5 ml) was heated at 60° C. for 6 h. Treatment with aq. NaOH yielded a precipitate, which then is purified by prep LC-MS to afford the title product (30 mg). ¹H NMR (DMSO-d₆) δ 7.07-7.20 (m, 5H), 7.40 (t, 1H), 7.64 (s, 1H), 7.67 (d, 1H): LC/MS: m/z 414 (M), 416 (M+2)

### d. N-{6-[2-(2-Bromo-phenylimino)-4-oxo-thiazolidin-5-ylidenemethyl]-1H-benzoimidazol-2-yl}-2-dimethylamino-acetamide

A mixture of 5-(2-amino-3H-benzoimidazol-5-ylmethylene)-2-(2-bromo-phenylimino)-thiazolidin-4-one (40 mg), dimethylaminoacetic acid (13 mg), HBTU (45 mg), and triethylamine (25 mg) in dry DMF (1 ml) was stirred at rt for 6 hours. It was washed with water and the formed yellowish solid was collected by filtration. Prep LC-MS purification afforded the title product (10 mg). ¹HNMR (DMSO-d₆) δ 2.30 (s, 6H), 3.24 (s, 2H), 7.10 (m, 2H), 7.29 (m, 1H), 7.39 (m, 1H), 7.46 (m, 1H), 7.63-7.68 (m, 3H): LC/MS: m/z 500 (M+1)

### Example 88

### Methyl (5-{(Z)-[2-[(2-bromophenyl)amino]-4-oxo-1,3-thiazol-5(4H)-ylidene]methyl}-1H-benzimidazol-2-yl)carbamate

A mixture of 2-(2-bromo-phenylamino)-5-(3,4-diamino-benzylidene)-thiazol-4-one (88 mg, 0.23 mmol) and 1,3-bis(methoxycarbonyl)methyl-2-thiopsuedourea (46 mg, 0.23 mmol) in dry methanol (1.5 mL) was heated overnight at 60° C. with a air-cooling condenser. The formed yellowish solid was filtered and then washed with H₂O and MeOH to provide the title product (39 mg). ¹H NMR (DMSO-d₆) 3.75 (s, 3H), 7.12-7.16 (m, 2H), 7.28 (d, 1H), 7.41-7.46 (m, 2H), 7.57 (s, 1H), 7.71 (d, 1H), 7.74 (s, 1H), 12.0 (brs, 2H).

### Example 89

### (5Z)-2-[(2-chlorophenyl)amino]-5-{[1-(3,3-dimethylbutyl)-1H-benzimidazol-6-yl]methylidene}-1,3-thiazol-4(5H)-one

(a) 3-[(3,3-Dimethylbutyl)amino]-4-nitrobenzonitrile. A mixture of 3-(methyloxy)-4-nitrobenzonitrile (1.0 g; 5.6 mmol.) and (3,3-dimethylbutyl)amine (1.5 g; 14.8 mmol.) was stirred and heated at 100° C. for 18 h. The mixture was cooled and the solid mass slurried with hexanes and filtered to afford the title compound (0.75 g; 54%) as a yellow solid. C₁₈H₁₇N₃O₂ requires: % C, 63.2; % H, 6.9; % N, 17.0; Found: % C, 63.1; % H, 6.7; % N, 16.7. 1H NMR (400 MHz, DMSO-d₆) δ ppm 0.97 (s, 9H) 1.53-1.60 (m, 2 H) 3.34-3.41 (m, 2H) 7.02 (dd, J=8.72, 1.64 Hz, 1 H) 7.59 (d, J=1.77 Hz, 1H) 8.04 (t, J=5.31 Hz, 1 H) 8.19 (d, J=8.59 Hz, 1 H).
(b) 4-Amino-3-[(3,3-dimethylbutyl)amino]benzonitrile. A solution of the compound from Example 89a) (0.69 g; 2.8 mmol.) in ethyl acetate/methanol (3:1) (50.0 mL) was hydrogenated over 10% palladium-on-carbon (100 mg) at room temperature and atmospheric pressure for 30 min. The mixture was filtered through a pad of celite and the filtrate evaporated to afford the title compound (0.60 g; 99%) as a tan oil. 1H NMR (400 MHz, DMSO-d₆) δ ppm 0.92 (s, 9 H) 1.62-1.66 (m, 2H) 3.13-3.21 (m, 2H) 6.88 (d, J=8.54 Hz, 1H) 7.31 (dd, J=8.54, 1.79 Hz, 1H) 7.48 (d, J=1.79 Hz, 1H) 8.0-10.5 (br s, 3H).
(c) 1-(3,3-Dimethylbutyl)-1H-benzimidazole-6-carbaldehyde. A solution of the compound from Example 89c) (600 mg; 2.8 mmol.) in formic acid (15.0 mL) was stirred and heated under reflux for 1 h. The solution was then cooled to room temperature for the addition of a 50% aqueous suspension of Raney-nickel (1.0 mL) and water (3.0 mL). The mixture was then stirred and heated at 100° C. for 30 min. The mixture was filtered through a pad of celite and evaporated. The residue was treated with water (10.0 mL) then basified with sat. aqu. Sodium hydrogen carbonate and extracted with ethyl acetate (3×50.0 mL). The organic layers were dried and evaporated and the residue purified by flash-chromatography (silica gel, 5% methanol in chloroform) to afford the title compound (380 mg) contaminated with approximately 20% of the [1-(3,3-dimethylbutyl)-1H-benzimidazol-6-yl]methanol. 1H NMR (400 MHz, CHLOROFORM-d) δ ppm 1.09 (s, 9H) 1.81-1.88 (m, 2H) 4.24-4.30 (m, 2H) 7.84 (dd, J=8.34, 1.52 Hz, 1H) 7.93 (d, J=8.34 Hz, 1H) 7.99 (d, J=0.76 Hz, 1H) 8.12 (s, 1H) 10.14 (s, 1H).
(d) (5Z)-2-[(2-chlorophenyl)amino]-5-{[1-(3,3-dimethylbutyl)-1H-benzimidazol-6-yl]methylidene}-1,3-thiazol-4(5H)-one. A solution of the compound from Example 89c) (138 mg; 0.60 mmol.), 2-[(2-chlorophenyl)amino]-1,3-thiazol-4(5H)-one (130 mg; 0.57 mmol.) and piperidine (70 μL; 0.70 mmol.) in ethanol (1.0 mL) was stirred and heated in a microwave reactor at 150° C. for 20 min. The mixture was cooled and filtered to afford the title compound (49.0 mg, 19%) as a pale-yellow powder. C₂₃H₂₃ClN₄OS requires: % C, 62.9; % H, 5.3; % N, 12.8; found: % C, 62.9; % H, 4.9; % N, 12.5. 1H NMR (400 MHz, DMSO-d₆) δ ppm 0.89 (s, 9H) 1.57-1.66 (m, 2 H) 4.19-4.29 (m, 2 H) 7.15 (dd, J=7.83, 1.26 Hz, 1H) 7.21 (td, J=7.77, 1.39 Hz, 1H) 7.37 (ddd, J=17.94, 8.21, 1.39 Hz, 2H) 7.54 (dd, J=8.08, 1.26 Hz, 1H) 7.66 (s, 1H) 7.73 (d, J=8.34 Hz, 1H) 7.86 (s, 1H) 8.39 (s, 1 H) 12.64 (s, 1 H).

### Example 90

### (5Z)-2-[(2,6-dichlorophenyl)amino]-5-{[1-(3,3-dimethylbutyl)D-1H-benzimidazol-6-yl]methylidene}-1,3-thiazol-4(5H)-one, trifluoroacetate salt

Following the procedure of Example 89d) except substituting 2-[(2,6-dichlorophenyl)amino]-1,3-thiazol-4(5H)-one for 2-[(2-chlorophenyl)amino]-1,3-thiazol-4(5H)-one followed by purification by chromatography (ODS silica, gradient 10-100% acetonitrile/water (0.1% TFA)), the title compound was prepared (69.0 mg, 20%). C₂₃H₂₂Cl₂N₄OS. C₂HF₃O₂ requires: % C, 51.1; % H, 3.9; % N, 9.5; found: % C, 50.9; % H, 3.8; % N, 9.4. 1H NMR (400 MHz, DMSO-d₆) δ ppm 0.90 (s, 9H) 1.61-1.69 (m, 2H) 4.28-4.39 (m, 2H) 7.23 (t, J=8.21 Hz, 1H) 7.51-7.59 (m, 3H) 7.77-7.87 (m, 2H) 7.95 (s, 1H) 8.87 (s,

### Example 91

### (5Z)-5-{[1-(2-cvclopropylethyl)-1H-benzimidazol-6-yl]methylidene}-2-[(2,6-difluorophenyl)amino]-1,3-thiazol-4(5H)-one

(a) 4-amino-3-[(2-cyclopropylethyl)amino]benzonitrile. A mixture of 3-(methyloxy)-4-nitrobenzonitrile (0.5 g; 2.8 mmol.) and (2-cyclopropylethyl)amine (0.24 g; 2.8 mmol.) in DMSO (2.5 mL) was stirred and heated in a microwave reactor at 125° C. for 90 min. The mixture turned bright orange and was diluted with ethyl acetate (20 mL) and washed with sat. aqu. sodium hydrogen carbonate (20 mL) and brine (20 mL). The organic layer was dried over MgSO₄, filtered and rotary evaporated down to residue. The crude residue was dissolved in methanol (10 mL) and ethyl acetate (10 mL) and treated with 10% palladium on carbon (20 mg) and hydrogenated at 25 psi for 1 h. The mixture was filtered through a pad of celite and the filtrate evaporated. Purification by flash-chromatography (silica gel, 5-50% ethyl acetate in hexanes) afforded the title compound (0.230 g; 41%) as a brown crystalline solid. C₁₂H₁₅N₃ MS(ES+) m/e 202 [M+H]⁺
(b) 1-(2-cyclopropylethyl)-1H-benzimidazole-6-carbaldehyde. A solution of the compound from Example 91a) (230 mg; 1.14 mmol.) in formic acid (7.0 mL) was stirred and heated under reflux for 2 h. The solution was then cooled to room temperature for the addition of a 50% aqueous suspension of Raney-nickel (1.0 mL) and water (1.0 mL). The mixture was then stirred and heated at 110° C. for 45 min. The mixture was cooled to 45° C. and then filtered through a pad of celite and evaporated. The residue was diluted with water (5.0 mL) then taken to pH=8 with sat. aqu. Sodium hydrogen carbonate and extracted with dichloromethane (2×25.0 mL). The organic layers were dried and evaporated to afford the title compound (236 mg;) as an impure oil that was used in the next step without further purification. MS(ES+) m/e 215 [M+H]⁺
(c) (5Z)-5-{[1-(2-cyclopropylethyl)-1H-benzimidazol-6-yl]methylidene}-2-[(2,6-difluorophenyl)amino]-1,3-thiazol-4(5H)-one. A solution of the compound from Example 91b) (236 mg; 1.10 mmol.), 2-[(2,6-difluorophenyl)amino]-1,3-thiazol-4(5H)-one (252 mg; 1.10 mmol.) and piperidine (109 μL; 1.10 mmol.) in ethanol (2.0 mL) was stirred and heated in a microwave reactor at 170° C. for 20 min. The mixture was diluted with ethyl acetate (20 mL) and water (10 mL). The organic layer was separated dried and filtered then purified by flash-chromatography (silica gel, 5-50% ethyl acetate in hexanes) to afford the title compound (50.0 mg, 11%) as a pale-yellow powder. C₂₂H₁₈F₂N₄OS MS(ES+) m/e 425 [M+H]⁺. 1H NMR (400 MHz, DMSO-d₆) δ ppm 12.95 (s, 1H) 8.91 (s, 1H) 8.09 (s, 1H) 8.00 (s, 1H) 7.91 (d, J=8.59 Hz, 1H) 7.54 (d, J=8.84 Hz, 1H) 7.29-7.39 (m, 3H) 4.48 (t, J=7.07 Hz, 2H) 1.75-1.82 (m, 2H) 0.69 (s, 1H) 0.37-0.42 (m, 2H) 0.01 (q, J=4.72Hz, 2H)

### Example 92

### (5Z)-5-{[1-(2-cyclohexylethyl)-1H-benzimidazol-6-yl]methylidene}-2-[(2,6-dichlorophenyl)amino]-1,3-thiazol-4(5H)-one, trifluoroacetate salt

(a) 2-(2-cyclohexylethyl)-1H-isoindole-1,3(2H)-dione. A solution of cyclohexylethanol (20.0 g; 0.156 mol.), triphenylphosphine (40.9 g; 1.1 equiv.) and phthalimide (22.9 g; 1.1 equiv.) in anhydrous tetrahydrofuran (300 mL) was stirred and cooled to 5° C. for the dropwise addition of diisopropyl azodicarboxylate (34.7 g; 1.1 equiv.) in anhydrous tetrahydrofuran (100 mL). The mixture was then stirred at room temperature for 18 h. The mixture was evaporated and the residue washed with diethyl ether (500 mL) and filtered, then the filtrate evaporated and purified by chromatography (silica gel, hexanes/ethyl acetate (4:1)) to afford the title compound (23.8 g; 6-0%) asa colorless oil. 1H NMR (400 MHz, CHLOROFORM-d) δ ppm 0.91-1.02 (m, J=12.06, 11.84, 11.84, 3.16Hz, 2H) 1.19-1.31 (m, 3H) 1.54-1.63 (m, 2H) 1.64-1.71 (m, 2H) 1.71-1.76 (m, 1H) 1.79 (s, 1H) 1.83 (d, J=2.02Hz, 1H) 3.69-3.74 (m, 2H) 7.72 (dd, J=5.56, 3.03Hz, 2H) 7.85 (dd, J=5.43, 2.91Hz, 2 H).
(b) (2-Cyclohexylethyl)amine, hydrochloride salt. A solution of the compound from Example 92a) (23.8 g; 0.092 mol.) and hydrazine hydrate (5.0 mL; 1.1 equiv.) in methanol (250 mL) was stirred and heated under reflux for 3 h. The mixture was cooled and evaporated and the residue slurried with diethyl ether (500 mL) and filtered. The filtrate was then evaporated and dissolved in diethyl ether (200 mL) then the solution saturated with gasous hydrochloric acid. The mixture was filtered to afford the title compound (1.7 g; 14%) as a yellow powder. 1H NMR (400 MHz, DMSO-d₆) δ ppm 0.83-1.67 (m, 13H), 2.72-2.80 (m, 2H), 8.02 (br s, 3H).
(c) 3-[(2-cyclohexylethyl)amino]-4-nitrobenzonitrile. A mixture of the compound from Example 92b) (1.23 g; 7.5 mmol.), 3-(methyloxy)-4-nitrobenzonitrile (1.12 g; 6.3 mmol.) and potassium carbonate (1.1 g; 8.0 mmol.) were well mixed then heated neat at 150° C. with stirring for 18 h. To the resulting cooled, solid mass was added 1M aqu. Hydrochloric acid (50.0 mL) and ethyl acetate (50.0 mL) and the mixture separated and the organic layer dried and evaporated. The residue was purified by chromatography (silica gel, hexanes/ethyl acetate (9:1)) to afford the title compound (0.27 g; 16%).
(d) (5Z)-5-{[1-(2-cyclohexylethyl)-1H-benzimidazol-6-yl]methylidene}-2-[(2,6-dichlorophenyl)amino]-1,3-thiazol-4(5H)-one, trifluoroacetate salt. Following the procedures of Examples 91b), 91c) and 91d) except substituting the compound from Example 92c) for the compound from Example 89a) and substituting 2-[(2,6-dichlorophenyl)amino]-1,3-thiazol-4(5H)-one for 2-[(2,6-difluorophenyl)amino]-1,3-thiazol-4(5H)-one followed by purification by chromatography (ODS silica, gradient 10-100% acetonitrile/water (0.1% TFA)), the title compound was prepared (21.1 mg). C₂₅H₂₄Cl₂N₄OS. C₂HF₃O₂ requires: % C, 52.9; % H, 4.1; % N, 9.1; found: % C, 53.4; % H, 4.3; % N, 9.1. 1H NMR (400 MHz, DMSO-d₆) δ ppm 0.85-0.95 (m, 2H ) 1.10-1.21 (m, 4H) 1.60-1.71 (m, 7H) 4.26-4.36 (m, 2H) 7.23 J=8.08 Hz, 1H) 7.42 (d, J=8.59Hz, 1H) 7.56 (d, J=8.08 Hz, 2H) 7.80 (d, J=8.34 Hz, 1 H) 7.92-7.96 (m, 2H) 8.77 (s, 1H) 12.93 (s, 1H).

### Example 93

### (2Z,5Z)-2-[(2,6-dichlorophenyl)imino]-5-[(2-phenyl-1H-benzimidazol-6-yl)methylidene]-1.3-thiazolidin-4-one, piperidine salt

(a) methyl 2-phenyl-1H-benzimidazole-6-carboxylate. A solution of benzaldehyde (2 mL, 20.0 mmol) and 40% aq. sodium hydrogen sulfite (21 mL) was stirred at room temperature for 1 h. To this mixture is added a solution of methyl 3,4-diaminobenzoate (3.32 g, 20.0 mmol) in ethanol (2 mL). The resulting solution is heated to reflux overnight. The mixture was diluted in water and the resulting precipitate was collected by filtration to obtain 4.90 g of the desired product in 97% yield. The crude was used without further purification. 1H NMR (400 MHz, CHLOROFORM-d) δ ppm 8.35 (s, 1H) 8.06-8.15 (m, 2H) 7.97 (dd, J=8.6, 1.5 Hz, 1H) 7.64 (s, 1 H) 7.41-7.50 (m, 3H).
(b) 2-phenyl-1H-benzimidazole-5-carbaldehyde. A solution of methyl 2-phenyl-1H-benzimidazole-5-carboxylate (0.300 g, 1.18 mmol) was treated with lithium aluminum hydride (2.4 mL, 2.38 mmol, 1 M solution in THF) under a nitrogen atmosphere at room temperature. The solution was stirred for 1 h and the dumped into ice, treated with saturated aq. ammonium chloride and diluted with brine. Extraction with three volumes of ethyl acetate afforded the title compound as a yellow solid in 54% yield [MS(ES+) m/e 225 [M+H]+. The crude was dissolved in acetone (15 mL) and immediately treated using manganese oxide (1.17 g, 13.4 mmol). The black solution was stirred at room temperature for 36 h. The residual black solid was filtered using a celite pad and washing with three volumes of acetone. The filtrate was concentrated under high vacuum to give a glue-like residue. The residue was washed with three volumes of ether to afford 0.139 g of the desired aldehyde as a yellow powder (52%). The crude material was used without further purification. [MS(ES+) m/e 223 [M+H]+. 1H NMR (400 MHz, MeOD-d₄) δ 10.04 (s, 1H) 8.17 (s, 1H) 8.09-8.16 (m, 2H) 7.86 (dd, J=8.3, 1.5Hz, 1H) 7.74 (d, J=8.3 Hz, 1 H) 7.54-7.61 (m, 3H).
(c) (2Z,5Z)-2-[(2,6-dichlorophenyl)imino]-5-[(2-phenyl-1H-benzimidazol-6-yl)methylidene)-1,3-thiazolidin-4-one. A microwave vial was charged with the compound from example 93b) (0.156 g, 0.702 mmol) and (2Z)-2-[(2,6-dichlorophenyl)imino]-1,3-thiazolidin-4-one (0.183 g, 0.702 mmol) in ethanol. The solution was treated with piperidine (0.07 mL, 0.702 mmol) and the contents were irradiated at 150° C. for 1 h in a microwave reactor. The mixture was allowed to cool to room temperature, taken up in water (15 mL) and extracted with ethyl acetate (3×10 mL). The organic layers were combined, dried over MgSO₄ and evaporated. The crude was purified by flash-chromatography (silica gel, 10% methanol in chloroform) to afford the title compound in 28% yield. [MS(ES+) m/e 465 [M+H]+. 1H NMR (400 MHz, DMSO-d₆) δ ppm 13.09 (s, 1H) 8.17 (d, J=7.3 Hz, 2H) 7.61 (s, 2H) 7.47-7.59 (m, 5H) 7.37 (m, 1H) 7.15 (s, 1H) 3.02 (m, 2H) 1.60-1.68 (m, 2H) 1.51-1.59 (m, 1H).

### Example 94

### (5Z)-2-[(2-chlorophenyl)amino]-5-{[1-(2-cyclopropylethyl)-1h-benzimidazol-6-yl]methylidene}-1,3-thiazol-4(5H)-one

Following the procedure of Example 91c) except substituting 2-[(2-chlorophenyl)amino]-1,3-thiazol-4(5H)-one for 2-[(2,6-difluorophenyl)amino]-1,3-thiazol-4(5H)-one followed by purification by flash-chromatography (silica gel, 5-100% ethyl acetate in hex), the title compound was prepared (81.0 mg, 16%). C₂₂H₁₉ClN₄OS MS(ES+) m/e 423 [M+H]⁺. 1H NMR (400 MHz, DMSO-d₆) δ ppm 12.70 (bs, 1H) 8.45 (s, 1H) 7.91-7.96 (m, 2H) 7.82 (d, J=8.59Hz, 1H) 7.64 (dd, J=7.96, 1.14 Hz, 1H) 7.43-7.50 (m, 1H) 7.40 (d, J=8.34 Hz, 1H) 7.28-7.34 (m, 1H) 7.25 (d, J=7.83Hz, 1H) 4.39 (t, J=6.95 Hz, 2H) 1.68-1.78 (m, 2H) 0.65 (ddd, J=12.19, 7.39, 4.93 Hz, 1H) 0.36-0.44 (m, 2H) −0.01 (q, J=4.80 Hz, 2H).

### Example 95

### (5Z)-5-{[1-(2-cyclopropylethyl)-1H-benzimidazol-6-yl]methylidene}-2-[(2,6-dichlorophenyl)amino]-1,3-thiazol-4(5H)-one

Following the procedure of Example 91c) except substituting 2-[(2,6-dichlorophenyl)amino]-1,3-thiazol-4(5H)-one for 2-[(2,6-difluorophenyl)amino]-1,3-thiazol-4(5H)-one followed by purification by chromatography (ODS silica, gradient 10-100% acetonitrile/water (0.1% TFA)), the title compound was prepared (74.0 mg, 18%). C₂₂H₁₈Cl₂N₄OS MS(ES+) m/e 456 [M+H]⁺. 1H NMR (400 MHz, DMSO-d₆) 1H NMR (400 MHz, DMSO-d₆) d ppm 12.98 (s, 1H) 8.79 (s, 1H) 8.04 (s, 1 H) 7.99 (s, 1H) 7.88 (d, J=8.59 Hz, 1 H) 7.65 (d, J=8.08 Hz, 2H) 7.47 (d, J=8.08 Hz, 1H) 7.32 (t, J=8.08 Hz, 1H) 4.45 (t, J=6.95 Hz, 2H) 1.73-1.81 (m, 2H) 0.67 (s, 1H) 0.35-0.42 (m, 2H) −0.00 (t, J=4.80 Hz, 2H)

### Example 96

### (2Z,5Z)-2-[(2,6-dichlorophenyl)imino]-5-{[2-(1-methylethyl)-1H-benzimidazol-6-yl]methylidene}-1,3-thiazolidin-4-one

(a) methyl 2-(1-methylethyl)-1H-benzimidazole-6-carboxylate. A solution of isobutyraldehyde (0.22 mL, 2.41 mmol) and 40% aq. sodium hydrogen sulfite (2.6 mL) was stirred at room temperature for 1 h. To this mixture is added a solution of methyl 3,4-diaminobenzoate (0.400 g, 2.41 mmol) in ethanol (2 mL). The resulting solution is heated to reflux overnight. The mixture was diluted in water and the resulting precipitate was collected by filtration to obtain 0.524 g of the desired product in >99% yield. The crude was used without further purification. [MS(ES+) m/e 219 [M+H]+. 1H NMR (400 MHz, CHLOROFORM-d) δ ppm 8.30 (d, J=1.0 Hz, 1H) 7.93 (dd, J=8.6, 1.5 Hz, 1H) 7.54 (d, J=8.6 Hz, 1H) 3.90 (s, 3H) 3.30-3.41 (m, 1H) 1.48 (d, J=7.1 Hz, 6H).
(b) [2-(1-methylethyl)-1H-benzimidazol-6-yl]methanol. A THF solution of the compound in example 96a) (0.524 g, 2.40 mmol) was treated with lithium aluminum hydride (4.8 mL, 4.80 mmol, 1 M solution in THF) under a nitrogen atmosphere at room temperature. The solution was stirred for 1 h and the dumped into ice, treated with saturated aq. ammonium chloride and diluted with brine. Extraction with three volumes of ethyl acetate afforded 0.354 g of the title compound (78%). [MS(ES+) m/e 191 [M+H]+. 1H NMR (400 MHz, METHANOL-d₄) δ ppm 7.53 (br. s., 1H) 7.44-7.51 (m, 1H) 7.20-7.24 (m, 1H) 4.69 (s, 2H) 3.16-3.27 (m, 1H) 1.44 (d, J=7.1 Hz, 6H).
(c) 2-(1-methylethyl)-1H-benzimidazole-6-carbaldehyde. A solution of the compound from example 96b) (0.354 g, 1.86 mmol) in acetone (5 mL) was treated with manganese oxide (1.60 g, 18.6 mmol). The solution was stirred overnight at room temperature. The mixture was filtered using a celite pad and was washed with acetone three times. The combined washings were combined to give 0.212 g of a white solid as the desired compound (61%). [MS(ES+) m/e 189 [M+H]+. 1H NMR (400 MHz, CHLOROFORM-d) δ ppm 10.06 (s, 1H) 8.11 (d, J=1.0 Hz 1H) 7.81 (dd, J=8.3 Hz, 1.5 Hz, 1H) 7.67 (d, J=8.4 Hz, 1H) 3.30-3.41 (m, 1H) 1.53 (d, J=7.0 Hz, 6H).
(d) (2Z,5Z)-2-[(2,6-dichlorophenyl)imino]-5-{[2-(1-methylethyl)-1H-benzimidazol-6-yl]methylidene}-1,3-thiazolidin-4-one. Following the procedure of Example 93c) except substituting 2-(1-methylethyl)-1H-benzimidazole-6-carbaldehyde (0.212 g, 1.13 mmol) for 2-phenyl-1H-benzimidazole-5-carbaldehyde followed by flash-chromatography (silica gel, 10% methanol in chloroform), the title compound was obtained as a yellow solid in 21% yield (0.104 g). [MS(ES+) m/e 431 [M+H]+. 1H NMR (400 MHz, DMSO-d₆) δ ppm 7.84 (br. s., 1H) 7.52-7.66 (m, 4H) 7.30 (dd, J=8.5 Hz, 1.3 Hz 1 H) 7.23 (t, J=8.1 Hz, 1H) 3.06-3.21 (m, 1H) 1.32 (d, J=6.8 Hz, 6H).

### Example 97

### (2Z,5Z)-2-[(2,6-dichlorophenyl)imino]-5-{[2-(2-methylpropyl)-1H-benzimidazol-6-yl]methylidene}-1,3-thiazolidin-4-one, piperidine salt

(a) methyl 2-(2-methylpropyl)-1H-benzimidazole-5-carboxylate. Following the procedure of Example 96a) except substituting isovaleraldehyde (0.258 g, 3.00 mmol) for isobutyraldehyde and employing aq work up with extraction using ethyl acetate, the title compound was prepared in >99% yield 0.759 g. [MS(ES+) m/e 233 [M+H]+.
(b) [2-(2-methylpropyl)-1H-benzimidazol-5-yl]methanol. Following the procedure employed in Example 96b) except substituting methyl 2-(2-methylpropyl)-1H-benzimidazole-5-carboxylate (0.759 g, 3.27 mmol) for methyl 2-(1-methylethyl)-1H-benzimidazole-6-carboxylate afforded the desired compound as a white solid (0.44 g) in 66% yield. [MS(ES+) m/e 205 [M+H]+.
(c) 2-(2-methylpropyl)-1H-benzimidazole-5-carbaldehyde. Following the procedure employed for Example 96c) but substituting [2-(2-methylpropyl)-1H-benzimidazol-5-yl]methanol (0.44 g, 2.15 mmol) for [2-(1-methylethyl)-1H-benzimidazol-6-yl]methanol afforded 0.395 g of the desired compound as a dark orange material in 92% yield. [MS(ES+) m/e 203 [M+H]+.
(d) (2Z,5Z)-2-[(2,6-dichlorophenyl)imino]-5-{[2-(2-methylpropyl)-1H-benzimidazol-6-yl]methylidene}-1,3-thiazolidin-4-one, piperidine salt. Following the procedure for Example 93c) except substituting 2-(2-methylpropyl)-1H-benzimidazole-5-carbaldehyde (0.395 g, 1.95 mmol) for 2-phenyl-1H-benzimidazole-5-carbaldehyde, the desired compound was obtained in 13% (0.11 g) as a yellow powder. [MS(ES+) m/e 445 [M+H]+. 1H NMR (400 MHz, METHANOL-d₄) δ ppm 7.65 (s, 2H) 7.50-7.56 (m, 1H) 7.43 (d, J=8.1 Hz, 2H) 7.32-7.38 (m, 1H) 7.12 (t, J=8.1 Hz, 1H) 3.09-3.14 (m, 4H) 2.75 (d, J=7.3 Hz, 2H) 2.24-2.16 (m, 1 H) 1.74-1.82 (m, 4H) 1.65-1.74 (m, 2H) 0.99 (d, J=6.8 Hz, 6H).

### Example 98

### (2Z,5Z)-2-[(2,6-dichlorophenyl)imino]-5-{[2-(3-pyridinyl)-1H-benzimidazol-6-yl]methylidene}-1,3-thiazolidin-4 one

(e) methyl 2-(3-pyridinyl)-1H-benzimidazole-5-carboxylate. The procedure for Example 96a) except substituting 3-pyridinecarbaldehyde (0.400 g, 3.70 mmol) for isobutyraldehyde was used. The filtrated residue was purified using flash-chromatography (silica gel, 10% methanol in chloroform) to yield 0.727 g, of a yellow solid (78%). [MS(ES+) m/e 254 [M+H]+.
(f) [2-(3-pyridinyl)-1H-benzimidazol-5-yl]methanol. A solution of the compound from Example 98a) (0.727 g, 2.87 mmol.) in THF was treated as the compound in Example 96b). The desired product was obtained as a yellow solid in 87% yield (0.560 g) and was used in the next step without further purification. [MS(ES+) m/e 226 [M+H]+.
(g) 2-(3-pyridinyl)-1H-benzimidazole-5-carbaldehyde. A procedure similar for Example 96c) except substituting [2-(3-pyridinyl)-1H-benzimidazol-5-yl]methanol (0.560 g, 2.49 mmol) was used to obtain the desired compound in 20% yield (0.104 g) as a yellow powder. [MS(ES+) m/e 224 [M+H]+.
(h) (2Z,5Z)-2-[(2,6-dichlorophenyl)imino]-5-{[2-(3-pyridinyl)-1H-benzimidazol-6-yl]methylidene}-1,3-thiazolidin-4-one. The procedure for Example 93c) was used except for substituting 2-(3-pyridinyl)-1H-benzimidazole-5-carbaldehyde (0.104 g, 0.466 mmol) instead of 2-phenyl-1H-benzimidazole-5-carbaldehyde. The title compound was obtained as a yellow solid in 28% yield (0.061 g) after work up and purification. [MS(ES+) m/e 466 [M+H]+. 1H NMR (400 MHz, DMSO-d₆) δ ppm 13.43 (d, J=46.5 Hz, 1H) 12.89 (br. s., 1H) 9.34 (br. s., 1 H) 8.65-8.75 (m, 1 H) 8.50 (s, 1H) 7.75-7.96 (m, 2H) 7.68 (s, 1 H) 7.54-7.64 (m, 3H) 7.42 (m, 1H) 7.25 (t, J=8.2 Hz, 1H).

### Example 99

### (2Z,5Z)-2-[(2,6-dichlorophenyl)imino]-5-{[2-(hydroxymethyl)-1H-benzimidazol-5-yl]methylidene}-1,3-thiazolidin-4-one

(a) methyl 2-({[(1,1-dimethylethyl)(dimethyl)silyl]oxy}methyl)-1H-benzimidazole-5-carboxylate. The procedure used for Example 96a) was used except substituting {[(1,1-dimethylethyl)(dimethyl)silyl]oxy}acetaldehyde (1.00 g, 5.70 mmol) for isobutyraldehyde. After diluting with water the mixture was extracted using three volumes of ethyl acetate. The combined organic portions were dried over magnesium sulfate and the whole was filtrated and concentrated. The crude was purified using flash chromatography (silica gel, 60% Ethyl acetate, hexane) to obtain a mixture of the desired TBS protected alcohol 99a (0.02 g) [MS(ES+) m/e 321 [M+H]+ and the deprotected alcohol 99a2) (0.386 g) for 22% yield [MS(ES+) m/e 207 [M+H]+.
(b) [2-({[(1,1-dimethylethyl)(dimethyl)silyl]oxy}methyl)-1H-benzimidazol-5-yl]methanol. A solution of the compound from Example 99a) (0.02 g, 0.062 mmol.) in dichloromethane (3.00 mL) was kept at −78° C. and treated with diisobutylaluminum hydride (0.075 mL, 0.075 mmol) dropwise. After 1 h, the solution was quenched with the methanol while keeping the temperature at −78° C. The cold bath is removed and the mixture was treated with sat. aq. Rochelle salt. The solution was stirred for 1 h, then extracted using three volumes of ethyl acetate, dried over magnesium sulfate, filtrated and concentrated to afford 58% yield. The crude was used without further purification in the next step. [MS(ES+) m/e 293 [M+H]+].
(c) 2-({[(1,1-dimethylethyl)(dimethyl)silyl]oxy}methyl)-1H-benzimidazole-5-carbaldehyde. Following the procedure for Example 96c) but substituting 99b) (0.02 g, 0.07 mmol) for the compound in 96c), the desired compound was obtained as an oil in >99% yield [MS(ES+) m/e 291 [M+H]+.
(d) (2Z,5Z)-2-[(2,6-dichlorophenyl)imino]-5-{[2-({[(1,1-dimethylethyl)(dimethyl)silyl]oxy}methyl)-1H-benzimidazol-5-yl]methylidene}-1,3-thiazolidin-4-one. A microwave vial was charged with 2-({[(1,1-dimethylethyl)(dimethyl)silyl]oxy}methyl)-1H-benzimidazole-5-carbaldehyde (0.0312 g, 0.11 mmol), (2Z)-2-[(2,6-dichlorophenyl)imino]-1,3-thiazolidin-4-one (0.029 g, 0.11 mmol), piperidine (0.02 mL, 0.11 mol) and ethanol (1 mL). The contents were irradiated at 150° C. for 1 h. The resulting solution was treated with 1 N HCl (5 ml) and the resulting precipitate was filtered, washed with 5×5 mL portions of water and dried under high vacuum to afford 0.032 g (55%) of the desired material. The crude was used without further purification. [MS(ES+) m/e 534 [M+H]+.
(e) (2Z,5Z)-2-[(2,6-dichlorophenyl)imino]-5-{[2-(hydroxymethyl)-1H-benzimidazol-5-yl]methylidene}-1,3-thiazolidin-4-one. A solution of the compound in Example 99d) (0.032 g, 0.06 mmol) was taken up in a 1:1 mixture of THF:water (2 mL) and was treated with aq. acetic acid (0.004 mL, 0.06 mL). The resulting solution was stirred overnight at room temperature. The mixture was diluted in 3 mL of ethyl acetate and extracted three times. The combined organic portions were washed with sat. aq. sodium bicarbonate, dried over magnesium sulfate, filtrated and concentrated to afford the title compound as a yellow solid in 38% yield (9.6 mg). [MS(ES+) m/e 419 [M+H]+. 1H NMR (400 MHz, METHANOL-d₄) δ ppm 7.85 (s, 1H) 7.69 (s, 1H) 7.61 (d, J=8.3 Hz, 1H) 7.44-7.51 (m, 2H) 7.39 (dd, J=8.5, 1.1 Hz, 1H) 7.18 (t, J=8.2 Hz, 1H) 4.84 (s, 2H).

### Example 100

### (2Z,5Z)-2-[(2,6-dichlorophenyl)imino]-5-{[2-(2-hydroxyethyl)-1H-benzimidazol-5-yl]methylidene}-1,3-thiazolidin-4-one

(a) methyl 2-(2-{[(1,1-dimethylethyl)(dimethyl)silyl]oxy}ethyl)-1H-benzimidazole-5-carboxylate. Using the procedure for Example 96a) except substituting 3-{[(1,1-dimethylethyl)(dimethyl)silyl]oxy}propanal (200 g, 10.6 mmol) for isobutyraldehyde, followed by purification using flash chromatography (silica gel, 60% ethyl acetate/hexane) the desired product 100a1) was obtained in 42% yield, along with the deprotected alcohol 100a2) (0.205 g, 8%). The protected alcohol 100a1) was carried on to the next step [MS(ES+) m/e 335 [M+H]+].
(b) [2-(2-{[(1,1-dimethylethyl)(dimethyl)silyl]oxy}ethyl)-1H-benzimidazol-5-yl]methanol. A solution of the compound in Example 100a1) (0.210 g, 0.0.63 mmol) was taken up in dichloromethane (3 mL) and treated with diisobutylaluminum hydride (0.63 mL, 0.63 mmol) at −78° C. The mixture was allowed to reach room temperature over 1 h. Then it was quenched with methanol (1 mL), treated with sat. aq. Rochelle salt (10 mL) and the slurry was stirred overnight. Extraction with 3×10 mL ethyl acetate, drying over magnesium sulfate and concentration under high vacuum afforded 0.25 g (>99%) of the title compound. The crude was used without further purification [MS(ES+) m/e 307 [M+H]+].
(c) 2-(2-{[(1,1-dimethylethyl)(dimethyl)silyl]oxy}ethyl)-1H-benzimidazole-5-carbaldehyde. Following the same procedure as in Example 96c) but substituting Example 100b) (0.25 g, 0.81 mmol) for the compound in Example 96c), The desired product was obtained 38% yield. The crude was used in the next step. [MS(ES+) m/e 305 [M+H+]
(d) (2Z,5Z)-2-[(2,6-dichlorophenyl)imino]-5-{[2-(2-hydroxyethyl)-1H-benzimidazol-5-yl]methylidene}-1,3-thiazolidin-4-one. A microwave vial was charged with the compound from Example 100c) (0.095 g, 0.31 mmol), (2Z)-2-[(2,6-dichlorophenyl)imino]-1,3-thiazolidin-4-one (0.082 g, 0.31 mmol), piperidine (0.03 mL, 0.31 mmol) and ethanol (3 mL). The vial was sealed and irradiated for 1 h at 150° C. in a microwave reactor. The crude was treated with aq. 1 N hydrochloric acid (3 mL) and the resulting precipitate was filtered off. The remaining solid was washed with water and dried under high vacuum. The desired compound was obtained after flash chromatography (silica gel, 10% methanol, dichloromethane) as a brown solid in 26% yield (0.035 g) [MS(ES+) m/e 433 [M+H]+. 1H NMR (400 MHz, DMSO-d₆) δ ppm 13.03 (br. s., 1H) 7.94 (s, 1H) 7.88 (s, 1H) 7.84 (d, J=8.6 Hz, 1H) 7.62 (d, J=7.6 Hz, 1H) 7.58 (d, J=8.1 Hz, 2H) 7.25 (t, J=8.2 Hz, 1H) 3.88 (t, J=5.7 Hz, 2H) 3.22(t, J=5.8 Hz, 2H).

### Example 101

### (2Z,5Z)-2-[(2,6-dichlorophenyl)imino]-5-{[2-(2-pyridinyl)-1H-benzimidazol-6-yl]methylidene}-1,3-thiazolidin-4-one

(a) methyl 2-(2-pyridinyl)-1H-benzimidazole-5-carboxylate. A solution of 2-pyridinecarbaldehyde (0.19 g, 1.80 mmol) in 40% aq. sodium hydrogen sulfite was stirred for 1 h at room temperature before being added to a solution of methyl 3,4-diaminobenzoate (0.300 g, 1.80 mmol) in ethanol (3 mL). The resulting mixture was stirred under reflux overnight. Then it was taken in 10 mL of water and extracted with 3×10 mL of ethyl acetate, dried over magnesium sulfate, filtrated and concentrated. Flash chromatography (silica gel, 10% methanol, dichloromethane) afforded the desired compound as white powder in very low yield (13%) [MS(ES+) m/e 254 [M+H]+].
(b) [2-(2-pyridinyl)-1H-benzimidazol-5-yl]methanol. Following the procedure from Example 96a) but substituting 101a) (0.058 g, 0.23 mmol) for 96a), the desired compound was obtained in quantitative yield. The crude was used in the following step [MS(ES+) m/e 226 [M+H]+].
(c) 2-(2-pyridinyl)-1H-benzimidazole-5-carbaldehyde. Following the same procedure used in Example 96c) but substituting 96b) with 101b) (0.052 g, 0.23 mmol), the desired product was obtained as a yellow solid in 40% yield. The crude was used without further purification [MS(ES+) m/e 224 [M+H]+].
(d) (2Z,5Z)-2-[(2,6-dichlorophenyl)imino]-5-{[2-(2-pyridinyl)-1H-benzimidazol-6-yl]methylidene}-1,3-thiazolidin-4-one. A microwave vial was charged with the compound from Example 100c) (0.02 g, 0.09 mmol), (2Z)-2-[(2,6-dichlorophenyl)imino]-1,3-thiazolidin-4-one (0.023 g, 0.09 mmol), piperidine (0.01 mL, 0.09 mL) and ethanol (2 mL). The vial was irradiated for 2 h at 150° C. in a microwave reactor. The solution was cooled to room temperature and diluted in water (4 mL). The aq. phase was extracted using 3×4 mL ethyl acetate. The combined organic portions were dried over magnesium sulfate, filtrated and concentrated to afford the desired product as a yellow solid in 81% yield. [MS(ES+) m/e 466 [M+H]+]. 1H NMR (400 MHz, DMSO-d₆) δ ppm 13.33 (d, J=71.7 Hz, 1 H) 12.88 (br. s., 1 H) 8.75 (s, 1 H) 8.31-8.36 (m, 1 H) 8.02 (t, J=7.7 Hz, 1H) 7.84-7.90 (m, 1H) 7.79 (d, J=8.3 Hz, 1H) 7.69 (s, 1H) 7.52-7.65 (m, 3 H) 7.42 (d, J=8.8 Hz, 1H) 7.25(t, J=8.1 Hz, 1H).

### Example 102

### (5Z)-5-{[1-(2-cyclopentylethyl)-1H-benzimidazol-6-yl]methylidene}-2-[(2,6-dichlorophenyl)amino]-1,3-thiazol-4(5H)-one

(a) 4-amino-3-[(2-cyclopentylethyl)amino]benzonitrile. A mixture of 3-(methyloxy)-4-nitrobenzonitrile (1.77 g; 9.9 mmol.) and (2-cyclopentylethyl)amine (4 mL; excess.) in DMSO (2.5 mL) was stirred and heated in a microwave reactor at 125° C. for 65 min. The mixture turned bright orange and was diluted with ethyl acetate (50 mL) and washed with sat. aqu. sodium hydrogen carbonate (50 mL) and brine (50 mL). The organic layer was dried over MgSO₄, filtered and rotary evaporated down to residue. Following purification by flash-chromatography (silica gel, 5-100% ethyl acetate in hexanes), the crude residue was dissolved in methanol (10 mL) and ethyl acetate (10 mL) and treated with 10% palladium on carbon (20 mg) and hydrogenated at 40 psi for 1 h. The mixture was filtered through a pad of celite and the filtrate evaporated to give the title compound (0.400 g; 18%) as a brown solid. MS(ES+) m/e 230 [M+H]+
(b) 1-(2-cyclopentylethyl)-1H-benzimidazole-6-carbaldehyde. A solution of the compound from Example 102a) (400 mg; 1.76 mmol.) in formic acid (10.0 mL) was stirred and heated under reflux for 2 h. The solution was then cooled to room temperature for the addition of a 50% aqueous suspension of Raney-nickel (2.0 mL) and water (2.0 mL). The mixture was then stirred and heated at 70° C. for 45 min. The mixture was cooled to 45° C. and then filtered through a pad of celite and evaporated. The residue was diluted with water (5.0 mL) then taken to pH=8 with sat. aqu. Sodium hydrogen carbonate and extracted with dichloromethane (2×25.0 mL). The organic layers were dried and evaporated with the major product being [1-(2-cyclopentylethyl)-1H-benzimidazol-6-yl]methanol. The crude alcohol was dissolved in acetone (5 mL), treated with manganese dioxide (300 mg) and stirred at room temperature for 3 h. The mixture was filtered through a pad of celite and evaporated to afford the title compound (300 mg;) as an oil that was used in the next step without further purification. MS(ES+) m/e 243 [M+H]+
(c) (5Z)-5-{[1-(2-cyclopentylethyl)-1H-benzimidazol-6-yl methylidene}-2-[(2,6-dichlorophenyl)amino]-1,3-thiazol-4(5H)-one. A solution of the compound from Example 102b) (120 mg; 0.496 mmol.), 2-[(2,6-dichlorophenyl)amino]-1,3-thiazol-4(5H)-one (129 mg; 0.496 mmol.) and piperidine (49 μL; 0.496 mmol.) in ethanol (2.0 mL) was stirred and heated in a microwave reactor at 150° C. for 20 min. The mixture was by purified directly by chromatography (ODS silica, gradient 10-100% acetonitrile/water (0.1% TFA)) to afford the title compound (21.0 mg, 8%) as a pale-yellow powder. C₂₄H₂₂Cl₂N₄OS MS(ES+) m/e 485 [M+H]⁺. 1H NMR (400 MHz, DMSO-d₆) δ ppm 12.91 (s, 1H) 8.72 (s, 1H) 7.92 (s, 2H) 7.80 (d, J=8.34 Hz, 1H) 7.56 (d, J=8.08 Hz, 2H) 7.40 (d, J=8.84 Hz, 1H) 7.23 (t, J=8.21 Hz, 1H) 4.29 (t, J=7.20 Hz, 2H) 1.76-1.84 (m, 2H) 1.67 (m, 2H) 1.51-1.60 (m, 3H) 1.40-1.49 (m, 2H) 1.07 (m, 2H)

### Example 103

### (5Z)-2-[(2-chlorophenyl)amino]-5-{[1-(2-cyclopentylethyl)-1H-benzimidazol-6-yl]methylidene}-1,3-thiazol-4(5H)-one

Following the procedure of Example 102c) except substituting 2-[(2-chlorophenyl)amino]-1,3-thiazol-4(5H)-one for 2-[(2,6-dichlorophenyl)amino]-1,3-thiazol-4(5H)-one, the title compound was prepared (15.0 mg, 8%). C₂₄H₂₃ClN₄OS MS(ES+) m/e 451 [M+H]⁺. 1H NMR (400 MHz, DMSO-d₆) δ ppm 12.88 (s, 1 H) 8.68 (s, 1 H) 7.92 (s, 1 H) 7.88 (s, 1H) 7.79 (d, J=8.08 Hz, 1H) 7.55 (d, J=7.33 Hz, 1H) 7.35-7.44 (m, 2H) 7.22 (s, 2H) 4.28 (s, 2 H) 1.79 (s, 2H) 1.67 (s, 3H) 1.55 (s, 2H) 1.44 (s, 2H) 1.07 (s, 2H)

### Example 104

### (2Z,5Z)-2-[(2,6-dichlorophenyl)imino]-5-[(2-methyl-1H-benzimidazol-6-yl)methylidene]-1,3-thiazolidin-4-one

(a) methyl 2-methyl-1H-benzimidazole-6-carboxylate. Following the procedure as in compound 101a) except substituting with acetaldehyde (0.10 mL, 1.81 mmol) instead of 2-pyridinecarbaldehyde, followed by flash chromatography (silica gel, 60% ethyl acetate, hexane) the desired compound was obtained as a yellow residue in 45% yield. [MS(ES+) m/e 191 [M+H]+].
(b) (2-methyl-1H-benzimidazol-6-yl)methanol. To a solution of 104a) (0.156 g, 0.820 mmol) in THF (5 mL) is held at 0° C. while adding lithium aluminum hydride (1.00 mL, 0.984 mmol). The solution was allowed to reach room temperature over 2 h. Then the mixture was dumped in water followed by treatment with Rochelle salt (5 mL). After extraction with ethyl acetate (3×10 mL) the washings were combined, dried and filtrated to yield 0.102 g (77%) of the desired compound. The crude was taken up to the next step. [MS(ES+) m/e 163 [M+H]+].
(c) 2-methyl-1H-benzimidazole-6-carbaldehyde. Following the procedure in Example 96c) but using 104b) (0.102 g, 0.63 mmol) instead of 96b), the desired compound was delivered in 60% yield. The crude was used without further purification. [MS(ES+) m/e 161 [M+H]+].
(d) (2Z,5Z)-2-[(2,6-dichlorophenyl)imino]-5-[(2-methyl-1H-benzimidazol-6-yl)methylidene]-1,3-thiazolidin-4-one. Following the procedure for Example 100d) but substituting 100c) with 2-methyl-1H-benzimidazole-6-carbaldehyde (0.069 g, 0.38 mmol) the desired compound was prepared in 73% yield as a yellow solid. [MS(ES+) m/e 403 [M+H]+]. 1H NMR (400 MHz, METHANOL-d₄) δ ppm 7.87 (s, 1 H) 7.79 (s, 1H) 7.75 (d, J=8.6 Hz, 1H) 7.62 (d, J=8.6 Hz, 1H) 7.47 (d, J=8.1 Hz, 2H) 7.19 (t, J=8.2 Hz, 1H) 2.81 (s, 3H).

### Example 105

### (2Z,5Z)-2-[(2,6-dichlorophenyl)imino]-5-[2-(4-pyridinyl)-1H-benzimidazol-5-yl]methylidene}-1,3-thiazolidin-4-one

(a) methyl 2-(4-pyridinyl)-1H-benzimidazole-5-carboxylate. Following the procedure for 101a) except substituting 2-pyridinecarbaldehyde with 4-pyridinecarbaldehyde (0.19 g, 1.77 mmol) the desired compound was obtained in 60% yield. The crude was used in the next step. [MS(ES+) m/e 254 [M+H]+].
(b) [2-(4-pyridinyl)-1H-benzimidazol-5-yl]methanol. The same procedure used in Example 104b) except substituting 104a) for 105a) (0.43 g, 1.70 mmol) afforded the desired compound in 46% yield. The crude was used without further purification. [MS(ES+) m/e 226 [M+H]+].
(c) 2-(4-pyridinyl)-1H-benzimidazole-5-carbaldehyde. The procedure used for Example 96c) but using 105b) (0.175 g, 0.78 mmol) instead of 96b) was employed to prepare the desired compound. After concentration the compound was obtained in 74% yield. The crude was carried on to the next step. [MS(ES+) m/e 224 [M+H]+].
(d) (2Z,5Z)-2-[(2,6-dichlorophenyl)imino]-5-{[2-(4-pyridinyl)-1H-benzimidazol-5-yl]methylidene}-1,3-thiazolidin-4-one. The procedure used to prepare Example 100) was used to prepare the desired compound. Substituting of 100c) for 105c) (0.065 g, 0.29 mmol) afforded 31% of a deep orange solid. [MS(ES+) m/e 466 [M+H]+]. 1H NMR (400 MHz, DMSO-d₆) δ ppm 12.94 (br. s., 1H) 8.88 (d, J=6.3 Hz, 2H) 8.28 (d, J=6.1 Hz, 2H) 7.93 (s, 1H) 7.79-7.85 (m, 2H) 7.60 (d, J=8.3 Hz, 2H) 7.45-7.51 (m, 1H) 7.26(t, J=8.2 Hz 1H)

### Example 106

### (2Z,5Z)-5-{[1-(2-cyclopropylethyl)-2-(3-pyridinyl)-1H-benzimidazol-6-yl]methylidene}-2-[2,6-dichlorophenyl)imino]-1,3-thiazolidin-4-one

(a) 3-[2-cyclopropylethyl)amino]-4-nitrobenzonitrile (0.430 g, 2.4 mmol) and charged with 3-(methyloxy)-4-nitrobenzonitrile (0.430 g, 2.4 mmol) and 2-cyclopropylethanamine (2.00 g, 24 mmol), sealed and irradiated at 125° C. for 4000 seconds. The solution was diluted in ethyl acetate (10 mL) and washed with sat. aq. sodium hydrogen carbonate. Flash chromatography yielded the desired compound as a bright orange solid in 56% yield. [MS(ES+) m/e 232 [M+H]+].
(b) 4-amino-3-[(2-cyclopropylethyl)amino]benzonitrile. A solution of 106a) (0.313 g, 1.35 mmol) in ethyl acetate (100 mL) was hydrogenated over 20% w/w palladium-on-carbon ((0.003 g, 0.27 mmol) at room temperature and 40 psi of hydrogen (g). The mixture was filtered through a celite pad and evaporated to afford the title compound as an orange oil (95%). The crude was used without further purification. [MS(ES+) m/e 202 [M+H]+].
(c) 1-(2-cyclopropylethyl)-2-(3-pyridinyl)-1H-benzimidazole-6-carbonitrile. The procedure used in Example 96a) except substituting isobutyraldehyde for 3-pyridinecarbaldehyde (0.139 mL, 1.48 mmol) and methyl 3,4-diaminobenzoate for 106b) (0.298 g, 1.48 mmol). The resulting precipitate was filtered off and washed with water. The phases were separated and the organic phase was dried over magnesium sulfate, filtrated and concentrated to give the desired product in 92% yield as a yellow solid. [MS(ES+) m/e 289 [M+H]+].
(d) 1-(2-cyclopropylethyl)-2-(3-pyridinyl)-1H-benzimidazole-6-carbaldehyde. A solution of 115c) (0.406 g, 1.41 mmol) in formic acid (10 mL) was treated with an aq. suspension of Raney-Ni (1 mL, 2800 slurry in water). The resulting mixture was refluxed for 1 h. The mixture was filtered, washed with ethanol, diluted in water (15 mL), alkalinized using portions of sodium carbonate, extracted with 3×15 mL dichloromethane, dried and concentrated. The desired aldehyde was obtained after flash chromatography (silica, 60% ethyl acetate, hexane) in 10% yield. [MS(ES+) m/e 292 [M+H]+].
(e) (2Z,5Z)-5-{[1-(2-cyclopropylethyl)-2-(3-pyridinyl)-1H-benzimidazol-6-yl]methylidene}-2-[(2,6-dichlorophenyl)imino]-1,3-thiazolidin-4-one. Following the procedure for Example 100d) except substituting 100c) for 106d) (0.040 g, 0.137 mmol) the desired compound was obtained as a yellow solid (0.028 g, 38%). 1H NMR (400 MHz, DMSO-d₆) δ ppm 12.94 (br. s., 1H) 8.79 (d, J=4.3 Hz, 1H) 8.34 (d, J=8.1 Hz, 1H) 8.06 (td, J=7.8, 1.8 Hz, 1H) 7.97 (br. s., 1H) 7.94 (s, 1H) 7.85 (d, J=8.3 Hz, 1H) 7.55-7.62 (m, 3H) 7.50 (d, J=8.1 Hz, 1H) 7.44 (d, J=8.3 Hz, 1H) 7.24 (t, J=8.2 Hz, 1H) 4.87 (t, J=7.1 Hz, 2H) 1.67 (q, J=7.1 Hz, 2H) 0.47-0.65 (m, 1H) 0.16-0.31 (m, 2 H) −0.11(q, J=4.9 Hz, 2H).

### Example 107

### (2Z,5Z)-2-[(2,6-dichlorophenyl)imino]-5-{[1-methyl-2-(3-pyridinyl)-1H-benzimidazol-5-yl]methylidene}-1,3-thiazolidin-4-one

(a) 1-methyl-2-(3-pyridinyl)-1H-benzimidazole-5-carbonitrile. The procedure used in Example 96a) except substituting methyl 3,4-diaminobenzoate for 4-amino-3-(methylamino)benzonitrile (0.102 g, 0.69 mmol) and isobutyraldehyde for 3-pyridinecarbaldehyde (0.06 mL, 0.69 mmol) was used to prepare the desired compound as a white solid in 80% yield. The crude was used without further purification. [MS(ES+) m/e 235 [M+H]+].
(b) 1-methyl-2-(3-pyridinyl)-1H-benzimidazole-5-carbaldehyde. A similar procedure used to prepare 115d) was employed except substituting 115c) with 117c) (0.130 g, 0.55 mmol). The crude was purified using flash chromatography (silica gel, 60% ethyl acetate, hexane) to yield a colorless oil as the desired product in 17% yield. The crude was immediately treated with manganese oxide (0.079 g, 0.91 mmol) at room temperature for 12 h. The mixture was filtered off through a celite pad. The filtrate was concentrated to afford the desired product as a colorless solid in quantitative yield. [MS(ES+) m/e 238 [M+H]+].
(c) (2Z,5Z)-2-[(2,6-dichlorophenyl)imino]-5-{[1-methyl-2-(3-pyridinyl)-1H-benzimidazol-6-yl]methylidene}-1,3-thiazolidin-4-one. The procedure used in Example 100d) except substituting 100c) for 107e) (0.023 g, 0.097 mmol) was used. After treatment with aq. 1 N hydrochloric acid and extraction with 3×5 mL ethyl acetate, drying over magnesium sulfate, filtration and concentration, the desired product was obtained as a deep orange solid in 27% yield. [MS(ES+) m/e 480 PJ+H]+]. 1H NMR (400 MHz, METHANOL-d₄) δ ppm 9.02 (br. s., 1H) 8.78 (br. s., 1H) 8.26-8.38 (m, 1 H) 7.66-7.81 (m, 3H) 7.57 (d, J=8.3 Hz, 2H) 7.37-7.52 (m, 4H) 7.17 (t, J=8.1 Hz, 1H) 3.38(d, J=7.3 Hz, 3H).

### Example 108

### (2Z,5Z)-5-{[2-(aminomethyl)-1H-benzimidazol-5-yl]methylidene}-2-[(2,6-dichlorophenyl)imino]-1,3-thiazolidin-4-one

(a) methyl 2-(azidomethyl)-1H-benzimidazole-5-carboxylate. A solution of the deprotected alcohol 99a2) (0.150 g, 0.73 mmol) in THF (10 mL) was treated with {[bis(phenyloxy)phosphanyl]oxy}azide (0.22 mL, 1.02 mmol) at 0° C. After 5 min 1,8-diazabicyclo[5.4.0]undec-7-ene (0.132 g, 0.88 mmol) was added and the mixture was stirred for 2 h at 0° C., then at room temperature for 20 h. The mixture was diluted in 10 mL of ethyl acetate, quenched using 10 mL of water and extracted 3×10 mL ethyl acetate. The organic layer was dried over sodium sulfate, filtrated and concentration. After purification (silica gel, 60% ethyl acetate, hexane) the desired product was obtained in quantitative yield. [MS(ES+) m/e 232 [M+H]+].
(b) methyl 2-(aminomethyl)-1H-benzimidazole-5-carboxylate. To a solution of 108a) (0.170 g, 0.737 mmol) in 12 mL of tetrahydrofuran was added triphenylphosphine (0.270 g, 1.03 mmol). After 2 min water (2 mL) was added and the mixture was stirred at room temperature for 3 h. Then it was treated with aq. 28% ammonium hydroxide (2 ml) and stirred for an additional 1 h. The mixture was separated into layers and the aq. portion was extracted with 3×10 mL of ethyl acetate. The combined organic portions were dried over magnesium sulfate, filtrated, concentrated and purified (silica gel, 60% ethyl acetate, hexane). The desired product was obtained in 38% yield. [MS(ES+) m/e 206 [M+H]+].
(c) 1,1-dimethylethyl {[5-(hydroxymethyl)-1H-benzimidazol-2-yl]methyl}carbamate. To a solution of 108b) (0.058 g, 0.28 mmol) in dimethylformamide (2 mL) was added BOC-anhydride (0.300 mL, 0.28 mmol) and triethylamine (0.04 mL, 0.28 mmol). The solution was irradiated at 150° C. for 300 sec in a microwave reactor (90% LCMS yield, [MS(ES+) m/e 306 [M+H]+]). The mixture was evaporated and the crude was immediately dissolved in 2 mL of tetrahydrofuran and treated with lithium aluminum hydride (0.30 mL, 0.28 mmol, 1 M solution in tetrahydrofuran). The mixture was stirred overnight and then quenched with 3 mL of methanol. The salts were washed with a sat. aq. solution of Rochelle salt (5 mL) and stirred for an additional 1 h. The solution was extracted using 3×10 mL ethyl acetate. The combined organic layers were dried over magnesium sulfate, filtrated and concentrated to give the desired product as a yellow oil in quantitative yield. 1H NMR (400 MHz, CHLOROFORM-d) δ ppm 7.29-7.36 (m, 1H) 7.08 (d, J=8.3 Hz, 1H) 6.33 (t, J=5.8 Hz, 1H) 4.65 (s, 2H) 4.41 (d, J=5.8 Hz, 2H) 2.06 (s, 1H) 1.38 (s, 9H).
(d) 1,1-dimethylethyl [(5-formyl-1H-benzimidazol-2-yl)methyl]carbamate. A solution of 108c) (0.09 g, 0.32 mmol) in ethyl acetate (5 mL) was treated with manganese oxide (0.28 g, 3.20 mmol) at room temperature for 2 h. The mixture was filtered using a celite pad. After concentration, the desired product was obtained as a colorless oil in 58% yield. [MS(ES+) m/e 276 [M+H]+].
(e) (2Z,5Z)-5-{[2-(aminomethyl)-1H-benzimidazol-5-yl]methylidene}-2-[(2,6-dichlorophenyl)imino]-1,3-thiazolidin-4-one. A microwave vial was charged with 108d) (0.047 g, 0.170 mmol), (2Z)-2-[(2,6-dichlorophenyl)imino]-1,3-thiazolidin-4-one (0.044 g, 0.170 mmol), piperidine (0.17 mL, 0.170 mmol), dissolved in ethanol (3 mL). The mixture was irradiated at 150° C. for 3600 sec, cooled and treated with aq. 1 N hydrochloric acid. The precipitate was collected by filtration, washed with water and dried under vacuum. The orange powder was immediately dissolved in ethyl acetate (5 mL) and treated with aq. 3 N hydrochloric acid for 18 h. The mixture was separated into layers by dissolving in 5 mL of water and extracted with 3×5 mL of ethyl acetate. The combined organic portions were neutralized using sat. aq. sodium hydrogen carbonate, the dried over magnesium sulfate, filtrated and concentrated to give a yellow solid as the final product (41% yield). [MS(ES+) m/e 418 [M+H]+]. 1H NMR (400 MHz, METHANOL-d₄) δ ppm 7.84 (s, 1H) 7.71 (s, 1H) 7.62 (s, 1H) 7.47 (d, J=8.3 Hz, 2H) 7.39 (s, 1H) 7.18 (t, J=8.2 Hz, 1 H) 4.65 (br. s., 2H).

### Example 109

### (2Z,5Z)-5-(1H-benzimidazol-5-ylmethylidene)-2-[(2,6-dichlorophenyl)imino]-1,3-thiazolidin-4-one

(a) methyl 1H-benzimidazole-5-carboxylate. Thionyl Chloride (0.135 mL, 1.85 mmol) was added dropwise to a solution of 1H-benzimidazole-5-carboxylic acid (0.300 g, 1.85 mmol) in methanol (50.0 mL). The solution was refluxed for 12 h and then cooled to room temperature. The mixture was slurried into sat. aq. sodium hydrogen carbonate, separated into layers and extracted using ethyl acetate (3×15 mL). The combined organic layers are dried over magnesium sulfate, filtrated and concentrated to give a purple solid as the desired product in 90% yield. The crude was used without further purification. [MS(ES+) m/e 177 [M+H]+].
(b) 1H-benzimidazol-5-ylmethanol. A solution of 109a) (0.056 g, 0.317 mmol) was treated as 104a) in Example 104b). The desired product was obtained as a pink oil in >99% yield. The crude was used immediately. [MS(ES+) m/e 149 [M+H]+].
(c) 1H-benzimidazole-5-carbaldehyde. A solution of 109b) (0.069 g, 0.466 mmol) in acetone was treated as 107d) in Example 107e). The desired product was obtained as a white powder in 48% yield. [MS(ES+) m/e 147 [M+H]+].
(d) (2Z,5Z)-5-(1H-benzimidazol-5-ylmethylidene)-2-[(2,6-dichlorophenyl)imino]-1,3-thiazolidin-4-one. Following the procedure in Example 100d) substituting 100c) for 109c) (0.033 g, 0.22 mmol). After filtration, washing and drying under vacuum the desired product was obtained as a bright yellow powder in 19% yield. [MS(ES+) m/e 389 [M+H]+]. 1H NMR (400 MHz, DMSO-d₆) δ ppm 12.94 (br. s., 1H) 8.87 (s, 1H) 7.91 (s, 1H) 7.85 (s, 1H) 7.80 (d, J=8.6 Hz, 1H) 7.58 (d, J=8.1 Hz, 2H) 7.52 (dd, J=8.6, 1.0 Hz, 1H) 7.24 (t, J=8.1Hz, 1H).

### Example 110

### (2Z,5Z)-2-[(2,6-dichlorophenyl)imino]-5-{[2-(hydroxymethyl)-1-methyl-1H-benzimidazol-6-yl]methylidene}-1,3-thiazolidin-4-one

(a) 3-methylamino)-4-nitrobenzoic acid. A microwave vial was charged with 3-(methyloxy)-4-nitrobenzoic acid (1.00 g, 5.07 mmol) and potassium carbonate (1.40 g, 10.1 mmol). Then water (2 mL) was added followed by methylamine (2.50 ml, 5.07 mmol, 2 M solution in methanol). The vial was irradiated at 160° C. during 300 seconds. The solution was allowed to reach room temperature and dissolved in ethyl acetate. The resulting precipitate was collected by filtration to give 68% of the desired product as a red-orange solid. The crude was used without further purification. [MS(ES+) m/e 197 [M+H]+].
(b) 4-amino-N-methyl-3-(methylamino)-N-(methyloxy)benzamide. A mixture of 110a) (0.552 g, 2.81 mmol) and thionyl chloride (1.65 mL) in excess was heated to 80° C. for 18 h and then concentrated under reduced pressure. Dry toluene was added and the mixture was evaporated (3 times). The acyl chloride was taken up in dichloromethane (20 mL) and the solution was cooled to 0° C. Then pyridine (0.68 mL, 8.43 mmol) and N,O-dimethylhydroxylamine hydrochloride (0.411 g, 4.22 mmol) were added and the solution was allowed to reach room temperature overnight. The resulting solution was diluted in dichloromethane, separated into layers and washed twice with brine. The combined organic washings were dried over sodium sulfate, filtrated and concentrated. The crude was immediately dissolved in ethanol (50 mL), transferred to a hydrogenation vessel and treated with 20% palladium-over-carbon (0.10 g, 0.10 mmol). The solution was purged under a stream of nitrogen and then exposed to 50 psi of hydrogen for 1.5 h. The mixture was filtrated and concentration under reduced pressure afforded the desired compound as a yellow oil in 58% yield. [MS(ES+) m/e 210 [M+H]+].
(c) 2-(hydroxymethyl)-N,1-dimethyl-N-(methyloxy)-1H-benzimidazole-6-carboxamide. A solution of sodium metabisulfite (0.10 g, 0.53 mmol) in water (0.5 mL) was introduced directly into a mixture of 110c) (0.22 g, 1.06 mmol) and {[(1,1-dimethylethyl)(dimethyl)silyl]oxy}acetaldehyde (0.20 mL, 1.06 mmol) in ethanol (5 mL). The solution was stirred overnight under reflux and then allowed to cool down to room temperature. The mixture was concentrated and the residue was extracted using ethyl acetate, dried over magnesium sulfate, filtrated and concentrated to give 4% of the desired alcohol as a clear oil. [MS(ES+) m/e 250 [M+H]+].
(d) 2-(hydroxymethyl)-1-methyl-1H-benzimidazole-6-carbaldehyde. To a solution of 110d) (0.012 g, 0.046 mmol) in dry tetrahydrofuran (1 mL) at −60° C. was added lithium aluminum hydride (0.05 mL, 0.046 mmol). Stirring was continued at −60° C. for 1 h and then methanol was added until bubbling ceased, followed by sat. aq. solution of Rochelle salt (2 mL). The solution was stirred overnight and then it was separated into layers and extracted using 3×5 mL ethyl acetate. The organic phase was dried over magnesium sulfate, filtrated and concentrated to give a yellow powder in 75% yield. 1H NMR (400 MHz, CHLOROFORM-d) δ ppm 10.14 (s, 1 H) 8.07 (s, 1H) 7.95 (s, 2H) 5.16 (s, 2H) 4.06(s, 3H).
(e) (2Z,5Z)-2-[(2,6-dichlorophenyl)imino]-5-{[2-(hydroxymethyl)-1-methyl-1H-benzimidazol-6-yl]methylidene}-1,3-thiazolidin-4-one. The procedure employed for Example 100d) was use but substituting 100c) for 110e) (0.006 g, 0.032 mmol). The expected product was obtained in 26% yield as a yellow solid. [MS(ES+) m/e 433 [M+H]+]. 1H NMP (400 MHζ, METHANOΛ-δ4) δ ppm 7.91 (s, 1H) 7.64-7.72 (m, 2H) 7.47 (d, J=8.1 Hz, 2H) 7.40 (d, J=8.6 Hz, 1H) 7.18 (t, J=8.2 Hz, 1H) 4.88 (s, 2H) 3.93(s, 3H).

### Example 111

### (5Z)-2-[(2,6-dichlorophenyl)amino]-5-({1-[2-(3-pyridinyl)ethyl]-1H-benzimidazol-6-yl}methylidene)-1,3-thiazol-4(5H)-one

(a) 4-amino-3-{[2-(3-pyridinyl)ethyl]amino}benzonitrile. A mixture of 3-(methyloxy)-4-nitrobenzonitrile (1.0 g; 5.62 mmol.) and [2-(3-pyridinyl)ethyl]amine (754 mg; 6.18 mmol.) in DMSO (1.0 mL) was stirred and heated in a microwave reactor at 125° C. for 65 min. The mixture turned bright brown-orange and a precipitate formed upon cooling and was filtered off and dried. The crude residue was dissolved in methanol (10 mL) and ethyl acetate (10 mL) and treated with 10% palladium on carbon (20 mg) and hydrogenated at 40 psi for 1 h. The mixture was filtered through a pad of celite and the filtrate evaporated to give the title compound (0.394 g; 29%) as a light brown solid which was used in the next step without further purification. MS(ES+) m/e 239 [M+H]⁺
(b) 1-[2-(3-pyridinyl)ethyl]-1H-benzimidazole-6-carbaldehyde. A solution of the compound from Example 111a) (394 mg; 1.66 mmol.) in formic acid (10.0 mL) was stirred and heated under reflux for 2 h. The solution was then cooled to room temperature for the addition of a 50% aqueous suspension of Raney-nickel (2.0 mL) and water (2.0 mL). The mixture was then stirred and heated at 70° C. for 45 min. The mixture was cooled to 45° C. and then filtered through a pad of celite and evaporated. The residue was diluted with water (5.0 mL) then taken to pH=8 with sat. aqu. sodium hydrogen carbonate and extracted with dichloromethane (2×25.0 mL). The organic layers were dried and evaporated to afford the title compound as a mixture with its corresponding alcohol as a minor product (222 mg) as an oil that was used in the next step without further purification. MS(ES+) m/e 252 [M+H]⁺.
(c) (5Z)-2-[(2,6-dichlorophenyl)amino]-5-({1-[2-(3-pyridinyl)ethyl]-1H-benzimidazol-6-yl}methylidene)-1,3-thiazol-4(5H)-one. A solution of the compound from Example 111b) (222 mg; 0.884 mmol.), 2-[(2,6-dichlorophenyl)amino]-1,3-thiazol-4(5H)-one (100 mg; 0.385 mmol.) and piperidine (38 μL; 0.385 mmol.) in ethanol (2.0 mL) was stirred and heated in a microwave reactor at 150° C. for 20 min. The mixture was by purified directly by chromatography (ODS silica, gradient 10-100% acetonitrile/water (0.1% TFA)) to afford the title compound (6.0 mg, 3%) as a pale-yellow powder. C₂₄H₁₇Cl₂N₅OS MS(ES+) m/e 493 [M+H]⁺. 1H NMR (400 MHz, DMSO-d₆) δ ppm 12.99 (bs, 1H) 8.64-8.73 (m, 3H) 8.09-8.16 (m, 2H) 7.89 (s, 1 H) 7.81 (d, J=8.59 Hz, 1 H) 7.70-7.76 (m, 1H) 7.57 (d, J=8.34 Hz, 2H) 7.37 (d, J=8.84 Hz, 1H) 7.23 (t, J=8.08 Hz, 1H) 4.65 (t, J=7.07 Hz, 2H) 3.31 (t, J=7.07 Hz, 2H)

### Example 112

### (5Z)-5-{[1-(cyclopropylmethyl)-1H-benzimidazol-6-yl]methylidene}-2-[(2,6-dichlorophenyl)amino]-1,3-thiazol-4(5-H)-one

(a) 4-amino-3-(cyclopropylmethylamino)benzonitrile. A mixture of 3-(methyloxy)-4-nitrobenzonitrile (1.0 g; 5.62 mmol.) and cyclopropylmethylamine (438 mg; 6.18 mmol.) in DMSO (1.0 mL) was stirred and heated in a microwave reactor at 125° C. for 65 min. The mixture turned bright orange and a precipitate formed upon cooling and was filtered off and dried. The crude residue was dissolved in methanol (10 mL) and ethyl acetate (10 mL) and treated with 10% palladium on carbon (20 mg) and hydrogenated at 40 psi for 1 h. The mixture was filtered through a pad of celite and the filtrate evaporated to give the title compound (0.317 g; 30%) as a light brown solid which was used in the next step without further purification. MS(ES+) m/e 188 [M+H]⁺
(b) 1-(cyclopropylmethyl)-1H-benzimidazole-6-carbaldehyde. A solution of the compound from Example 112a) (317 mg; 1.67 mmol.) in formic acid (10.0 mL) was stirred and heated under reflux for 2 h. The solution was then cooled to room temperature for the addition of a 50% aqueous suspension of Raney-nickel (2.0 mL) and water (2.0 mL). The mixture was then stirred and heated at 70° C. for 45 min. The mixture was cooled to 45° C. and then filtered through a pad of celite and evaporated. The residue was diluted with water (5.0 mL) then taken to pH=8 with sat. aqu. Sodium hydrogen carbonate and extracted with dichloromethane (2×25.0 mL). The organic layers were dried and evaporated to afford the title compound as a mixture with its corresponding alcohol as a minor product (182 mg) as an oil that was used in the next step without further purification. MS(ES+) m/e 201 [M+H]⁺
(c) (5Z)-5-{[1-(cyclopropylmethyl)-1H-benzimidazol-6-yl]methylidene}-2-[(2,6-dichlorophenyl)amino]-1,3-thiazol-4(5H)-one. A solution of the compound from Example 112b) (182 mg; 0.910 mmol.), 2-[(2,6-dichlorophenyl)amino]-1,3-thiazol-4(5H)-one (100 mg; 0.385 mmol.) and piperidine (38 μL; 0.385 mmol.) in ethanol (2.0 ml) was stirred and heated in a microwave reactor at 150° C. for 20 min. The mixture was by purified directly by chromatography (ODS silica, gradient 10-100% acetonitrile/water (0.1% TFA)) to afford the title compound (4.0 mg, 3%) as a orange powder. C₂₁H₁₆Cl₂N₄OS MS(ES+) m/e 443 [M+H]⁺.

### Example 113

### (5Z) -5-[(1-cyclopentyl-1H-benzimidazol-6-yl)methylidene]-2-[(2,6-dichlorophenyl)amino]-1,3-thiazol-4(5H)-one

(a) 4-amino-3-(cyclopentylamino)benzonitrile. A mixture of 3-(methyloxy)-4-nitrobenzonitrile (1.0 g; 5.62 mmol.) and cyclopentylamine (526 mg; 6.18 mmol.) in DMSO (1.0 mL) was stirred and heated in a microwave reactor at 125° C. for 65 min. The mixture turned orange and a precipitate formed upon cooling and was filtered off and dried. The crude residue was dissolved in methanol (10 mL) and ethyl acetate (10 mL) and treated with 10% palladium on carbon (20 mg) and hydrogenated at 40 psi for 1 h. The mixture was filtered through a pad of celite and the filtrate evaporated to give the title compound (0.331 g; 29%) as a light brown solid which was used in the next step without further purification. MS(ES+) m/e 202 [M+H]⁺
(b) 1-cyclopentyl-1-benzimidazole-6-carbaldehyde. A solution of the compound from Example 113a) (331 mg; 1.65 mmol.) in formic acid (10.0 mL) was stirred and heated under reflux for 2 h. The solution was then cooled to room temperature for the addition of a 50% aqueous suspension of Raney-nickel (2.0 mL) and water (2.0 mL). The mixture was then stirred and heated at 70° C. for 45 min. The mixture was cooled to 45° C. and then filtered through a pad of celite and evaporated. The residue was diluted with water (5.0 mL) them taken to pH=8 with sat. aqu. Sodium hydrogen carbonate and extracted with dichloromethane (2×25.0 mL). The organic layers were dried and evaporated to afford the title compound as a mixture with its corresponding alcohol as a minor product (206 mg) as an oil that was used in the next step without further purification. MS(ES+) m/e 215 [M+H]⁺
(c) (5Z )-5-[(1-cyclopentyl-1H-benzimidazol-6-yl)methylidene]-2-[(2,6-dichlorophenyl)amino]-1,3-thiazol-4(5H)-one. A solution of the compound from Example 111b) (206 mg; 0.962 mmol.), 2-[(2,6-dichlorophenyl)amino]-1,3-thiazol-4(5H)-one (100 mg; 0.385 mmol.) and piperidine (38 μL; 0.385 mmol.) in ethanol (2.0 mL) was stirred and heated in a microwave reactor at 150° C. for 20 min. The mixture was by purified directly by chromatography (ODS silica, gradient 10-100% acetonitrile/water (0.1% TFA)) to afford the title compound (5.0 mg, 3%) as a yellow powder. C₂₂H₁₈Cl₂N₄OS MS(ES+) m/e 457 [M+H]⁺.

### Example 114

### (5Z)-5-(1,3-Benzoxazol-6-ylmethylidene)-2-[(2,6-dichlorophenyl)amino]-1,3-thiazol-4(5H)-one

(f) (5Z)-2-[(2,6-Dichlorophenyl)amino]-5-[(3-hydroxy-4-nitrophenyl)methylidene]-1,3-thiazol-4(5H)-one. A mixture of 2-[(2,6-dichlorophenyl)amino]-1,3-thiazol-4(5H)-one (2.61 g; 0.01 mol.), 3-hydroxy-4-nitrobenzaldehyde (1.67 g; 0.01 mol.) and piperidine (1.0 mL; 0.01 mol.) in ethanol (5.0 mL) was stirred and heated in a microwave reactor at 150° C. for 20 min. The mixture was cooled and poured into 1M aqu. hydrochloric acid (50.0 mL) then extracted with ethyl acetate (200 mL). The organic layer was dried and evaporated to afford the title compound (3.2 g; 78%) as an orange powder. C₁₆H₉Cl₂N₃O₄S requires: % C, 46.9;% H, 2.2; % N, 10.2; found: % C, 46.9; % H, 2.1; % N, 10.0. 1H NMR (400 MHz, DMSO-d₆) δ ppm 7.14 (dd, J=8.59, 1.52 Hz, 1H) 7.20 (d, J=1.52 Hz, 1H) 7.24 (t, J=8.21 Hz, 1H) 7.58 (d, J=8.08 Hz, 2H) 7.69 (s, 1H) 7.94 (d, J=8.59 Hz, 1H) 11.31 (s, 1H) 13.11 (s, 1H).
(g) (5Z)-5-[(4-Amino-3-hydroxyphenyl)methylidene]-2-[(2,6-dichlorophenyl)amino]-1,3-thiazol-4(5H)-one. A solution of the compound from Example 113a) (3.1 g; 7.5 mmol.) in methanol (200 mL) was hydrogenated over 10% w/w palladium-on-carbon (0.60 g) at room temperature and atmospheric pressure for 20 h. The mixture was filtered through celite and evaporated to afford the title compound (2.85 g; quantitiative). 1H NMR (400 MHz, DMSO-d₆) δ ppm 5.46 (s, 2H) 6.61 (d, J=8.34 Hz, 1H) 6.74 (s, 1H) 6.83 (d, J=8.34 Hz, 1H) 7.19 (s, 1H) 7.35-7.45 (m, 1H) 7.53 (d, J=7.33 Hz, 2H) 9.49 (s, 1H) 12.55 (s, 1H).
(h) (5Z)-5-(1,3-Benzoxazol-6-ylmethylidene)-2-[(2,6-dichlorophenyl)amino]-1,3-thiazol-4(5H)-one. A solution of the compound from Example 113b) (0.42 g; 1.1 mmol.) in triethyl orthoformate (2.0 mL) was stirred and heated in a microwave reactor at 125° C. for 15 min. The mixture was cooled and directly purified by chromatography (silica gel, hexanes/ethyl acetate (7:3)) to afford the title compound (161 mg; 38%) as a cream powder. C₁₇H₉Cl₂N₃O₂S requires: % C, 52.3; % H, 2.3; % N, 10.8; found: % C, 52.0; % H, 2.3; % N, 10.1. 1H NMR (400 MHz, DMSO-d₆) δ ppm 7.24 (t, J=8.08 Hz, 1H) 7.51 (dd, J=8.34, 1.26 Hz, 1H) 7.58 (d, J=8.08 Hz, 2H) 7.86-7.94 (m, 2H) 8.00 (d, J=1.26 Hz, 1H) 8.88 (s, 1H) 13.00 (s, 1H).

### Example 115

### Capsule Composition

An oral dosage form for administering the present invention is produced by filing a standard two piece hard gelatin capsule with the ingredients in the proportions shown in Table I, below.

TABLE I

| INGREDIENTS | AMOUNTS |
|---|---|
| (5Z)-2-[(2-Chlorophenyl)amino]- | 25 mg |
| 5-[(1-methyl-1H-benzimidazol-6- | |
| yl)methylidene]-1,3-thiazol-4(5H)- | |
| one (compound of Ex. 1) | |
| Lactose | 55 mg |
| Talc | 16 mg |
| Magnesium Stearate | 4 mg |

### Example 116

### Injectable Parenteral Composition

An injectable form for administering the present invention is produced by stirring 1.5% by weight of (5Z)-2-[(2-Chlorophenyl)amino]-5-[(1,2-dimethyl-1H-benzimidazol-6-yl)methylidene]-1,3-thiazol-4(5H)-one (compound of Ex. 9) in 10% by volume propylene glycol in water.

### Example 117

### Tablet Composition

The sucrose, calcium sulfate dihydrate and an Akt inhibitor as shown in Table II below, are mixed and granulated in the proportions shown with a 10% gelatin solution. The wet granules are screened, dried, mixed with the starch, talc and stearic acid;, screened and compressed into a tablet.

TABLE II

| INGREDIENTS | AMOUNTS |
|---|---|
| (5Z)-2-[(2,6-dichlorophenyl)amino]- | 20 mg |
| 5-({1-[2-(3-pyridinyl)ethyl]-1H- | |
| benzimidazol-6-yl}methylidene)- | |
| 1,3-thiazol-4(5H)-one | |
| (compound of Ex. 111) | |
| calcium sulfate dihydrate | 30 mg |
| sucrose | 4 mg |
| starch | 2 mg |
| talc | 1 mg |
| stearic acid | 0.5 mg |

Biological Methods and Data

As demonstrated by the representative compounds of the present invention in Table 1, the compounds of the present invention have valuable pharmacological properties due to their potent ability to inhibit the hYAK3 kinase enzyme.

Substrate phosphorylation assays were carried out as follows:

YAK3 Scintillation Proximity Assays Using Ser164 of Myelin Basic Protein as the Phosphoacceptor

The source of Ser164 substrate peptide The biotinylated Ser164, S164A peptide(Biotinyl-LGGRDSRAGS*PMARR-OH), sequence derived from the C-terminus of bovine myelin basic protein (MBP) with Ser162 substituted as Ala 162, was purchased from California Peptide Research Inc. (Napa, Calif.), and its purity was determined by HPLC. Phosphorylation occurs at position 164 (marked S* above). The calculated molecular mass of the peptide was 2166 dalton. Solid sample was dissolved at 10 mM in DMSO, aliquoted, and stored at −20° C. until use.

The source of enzyme:

hYAK3: Glutathione-S-Transferase (GST)-hYak3-His6 containing amino acid residues 124-526 of human YAK3 (aa 124-526 of SEQ ID NO 2. in U.S. Pat. No. 6,323,318) was purified from baculovirus expression system in Sf9 cells using Glutathione Sepharose 4B column chromatography followed by Ni-NTA-Agarose column chromatography. Purity greater than 65% typically was achieved. Samples, in 50 mM Tris, 150 mM NaCl, 10% glycerol, 0.1% Triton, 250 mM imidazole, 10 mM β-mercapto ethanol, pH 8.0. were stored at −80° C. until use.

Kinase assay of purified hYAK3: Assays were performed in 96 well (Costar, Catalog No. 3789) or 384 well plates (Costar, Catalog No. 3705). Reaction (in 20, 25, or 40 μl volume) mix contained in final concentrations 25 mM Hepes buffer, pH 7.4; 10 mM MgCl₂; 10 mM β-mercapto ethanol; 0.0025% Tween-20; 0.001 mM ATP, 0.1 μCi of [γ-³³P]ATP; purified hYAK3 (7-14 ng/assay; 4 nM final); and 4 μM Ser164 peptide. Compounds, titrated in DMSO, were evaluated at concentrations ranging from 50 μM to 0.5 nM. Final assay concentrations of DMSO did not exceed 5%, resulting in less than 15% loss of YAK3 activity relative to controls without DMSO. Reactions were incubated for 2 hours at room temperature and were stopped by a 75 ul addition of 0.19 μg Streptavidin Scintillation Proximity beads (Amersham Pharmacia Biotech, Catalog No. RPNQ 0007) in PBS, pH 7.4, 10 mM EDTA, 0.1% Triton X-100, 1 mM ATP. Under the assay conditions defined above, the Kₘ(apparent) for ATP was determined to be 7.2+/−2.4 μM.

TABLE 1

| Compounds from example nos. | pIC₅₀ values |
|---|---|
| 59 | +++ |
| 58 | ++ |
| 53 | + |

| | |
|---|---|
| Legend | |
| pIC₅₀ values | Symbol |
| 8.99-8 | +++ |
| 7.99-7 | ++ |
| 6.99-6 | + |

pIC₅₀ = −log₁₀(IC₅₀)

The above biological data clearly shows that the compounds of formula I are useful for treating or preventing disease states in which hYAK3 proteins are implicated, especially diseases of the erythroid and hematopoietic systems, including but not limited to, anemias due to renal insufficiency or to chronic disease, such as autoimmunity, HIV, or cancer, and drug-induced anemias, myelodysplastic syndrome, aplastic anemia, myelosuppression, and cytopenia.

The compounds of formula I are especially useful in treating diseases of the hematopoietic system, particularly anemias. Such anemias include an anemia selected from the group comprising: aplastic anemia and myelodysplastic syndrome. Such anemias also include those wherein the anemia is a consequence of a primary disease selected from the group consisting of: cancer, leukemia and lymphoma. Such anemias also include those wherein the anemia is a consequence of a primary disease selected from the group consisting of: renal disease, failure or damage. Such anemias include those wherein the anemia is a consequence of chemotherapy or radiation therapy, in particular wherein the chemotherapy is chemotherapy for cancer or AZT treatment for HIV infection. Such anemias include those wherein the anemia is a consequence of a bone marrow transplant or a stem cell transplant. Such anemias also include anemia of newborn infants. Such anemias also include those which are a consequence of viral, fungal, microbial or parasitic infection.

The compounds of formula I are also useful for enhancing normal red blood cell numbers. Such enhancement is desirable for a variety of purposes, especially medical purposes such as preparation of a patient for transfusion and preparation of a patient for surgery.

While the preferred embodiments of the invention are illustrated by the above, it is to be understood that the invention is not limited to the precise instructions herein disclosed and that the right to all modifications coming within the scope of the following claims is reserved.

1-10. (canceled)

11. A compound of the Formula I, and/or a pharmaceutically acceptable salt, hydrate, solvate, or pro-drug thereof, 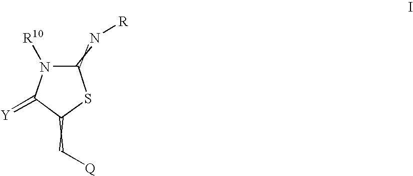
wherein:
R is selected from: hydrogen, aryl, substituted aryl, cycloalkyl, substituted cycloalkyl, C₁₋₆alkyl and substituted C₁₋₆alkyl;
R¹⁰ is selected from: hydrogen, C₁₋₆alkyl, —(CH₂)ₘOH and —(CH₂)ₘCOOH,
where m is 0 to 6;
Y is selected from: ═O, ═S and ═NR¹¹,
where R¹¹ is selected from: hydrogen, C₁₋₆alkyl, —(CH₂)ₚOH and —(CH₂)ₚCOOH,
where p is 0 to 6; and
Q is a radical or substituted radical of the formula, 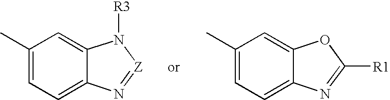
in which Z is N or C—R²;
wherein R² is hydrogen, —NH₂, —C₁₋₆alkyl, substituted -C₁₋₆alkyl, —CF₃, aryl or a radical or substituted radical of the formula 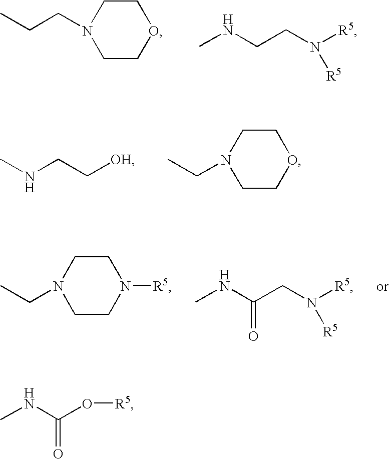
wherein R⁵ is selected from: hydrogen, -C₁₋₆alkyl and substituted -C₁₋₆alkyl; and R³ is hydrogen, —C₁₋₆alkyl, substituted -C₁₋₆alkyl or C₃₋₁₂cycloalkyl; and R¹ is hydrogen, -C₁₋₆alkyl, substituted -C₁₋₆alkyl, amino, mono substituted amino, disubstituted amino and trifluoromethyl.

12. A compound of claim 11, wherein the compound is a compound of Formula III 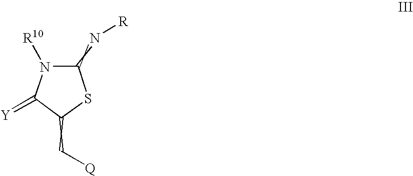
wherein:
R is selected from: hydrogen, C₁-C₁₂aryl, substituted C₁-C₁₂aryl, cycloalkyl, substituted cycloalkyl, C₁₋₆alkyl, —(CH₂)ₙ—NR^{k}R^{h}, —C(═NH)NH₂, —(CH₂)₂N(CH₃)₂, —C(═O)CH₃, —(CH₂)₂OCH₃, —(CH₂)₂OH, —(CH₂)₂C(CH₃)₃ and —(CH₂)CH(CH₃)₂, —C(═O)Ph, —C(═O)CH₂NHBOC, —(CH₂)₂CH(CH₃)₂,
where n is 0 to 6, and
R^{k} and R^{h} are independently selected form hydrogen, C₁₋₆alkyl and substituted C₁₋₆alkyl;
R¹⁰ is selected from: hydrogen, C₁₋₆alkyl, —(CH₂)ₘOH and —(CH₂)ₘCOOH,
where m is 0 to 6;
Y is selected from ═O, ═S and ═NR¹¹,
where R¹¹ is selected from: hydrogen, C₁₋₆alkyl, —(CH₂)ₚOH and —(CH₂)ₚCOOH,
where p is 0 to 6; and
Q is a radical of the formula, 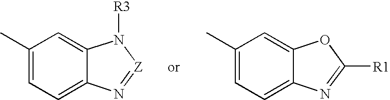
in which Z is N or C—R²,
wherein R² is hydrogen, —NH₂, —C₁₋₆alkyl, substituted -C₁₋₆alkyl, —CF₃, aryl or a radical of the formula 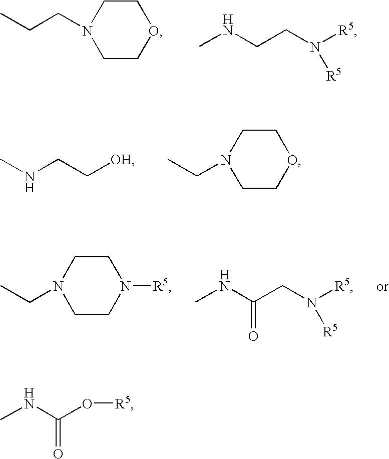
wherein R⁵ is selected from: hydrogen, —C₁₋₆alkyl and substituted -C₁₋₆alkyl; and
R³ is hydrogen, -C₁₋₆alkyl, substituted -C₁₋₆alkyl or C₃₋₁₂cycloalkyl; and
R¹ is hydrogen, -C₁₋₆alkyl, substituted -C₁₋₆alkyl, amino, mono substituted amino, disubstituted amino and trifluoromethyl,
and/or a pharmaceutically acceptable salt, hydrate, solvate, or pro-drug thereof.

13. A compound of claim 11, wherein the compound is a compound of Formula IV 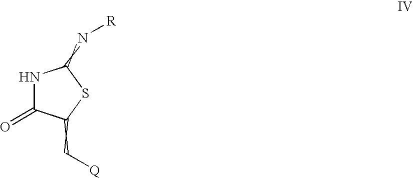
in which
R is 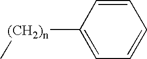
in which the phenyl radical is optionally and independently substituted with up to three substituents selected form: halogen, —C₁₋₆alkyl, —OC₁₋₆alkyl, —CF₃, —CN, —CO₂H, —SO₂NH₂, —CONH₂; or
R is a radical of the formula 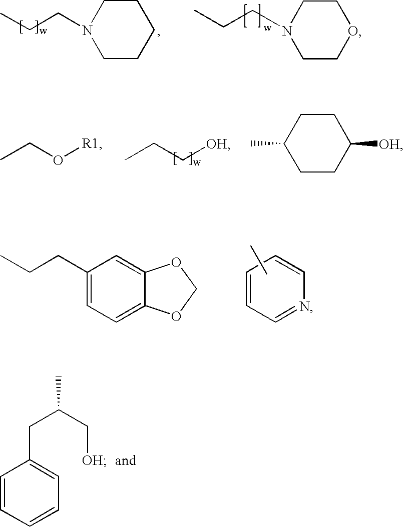
Q is a radical of the formula 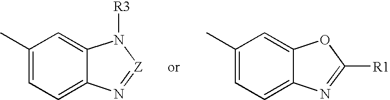
in which Z is N or C—R2;
wherein R2 is hydrogen, —NH₂, -C₁₋₆alkyl, —CF₃, or a radical of the formula 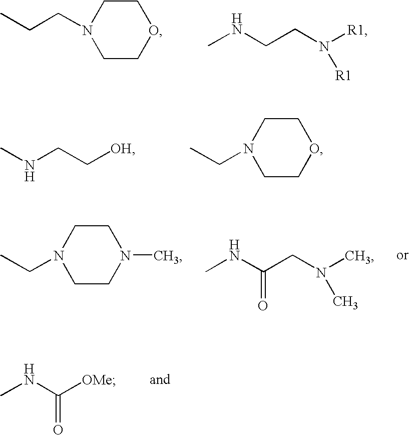
R3 is -C₁₋₆ alkyl, or a radical of the formula 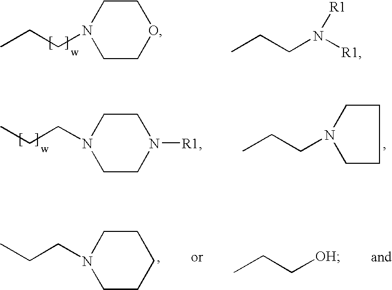
n equals zero to two;
w equals one to two; and
R1 is -C₁₋₆alkyl,
and/or a pharmaceutically acceptable salt, hydrate, solvate, or pro-drug thereof.

14. A compound of claim 13 in which R is phenyl optionally and independently substituted with up to three substituents selected from: hydrogen, -C₁₋₆alkyl, —OC₁₋₆alkyl, —CF₃, —CN, —CO₂H, —SO₂NH₂ and —CONH₂.

15. A compound of claim 14 in which R is 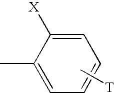
in which X is halogen or CF3; and Y is hydrogen, halogen, -C₁₋₆alkyl, —OC₁₋₆alkyl, —CF₃, —CN, —CO₂H, —SO₂NH₂, —CONH₂.

16. A compound of claim 15 in which Q is 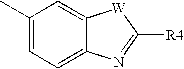
in which R4 is methyl or hydrogen, and W is O or N—R1, in which R1 is -C₁₋₆alkyl.

17. A method of inhibiting hYAK3 in a mammal; comprising, administering to the mammal a therapeutically effective amount of a compound of claim 11, or a pharmaceutically acceptable salt, hydrate, solvate or pro-drug thereof

18. A method of treating or preventing diseases of the erythroid and hematopoietic systems, caused by the hYAK3 imbalance or inappropriate activity; comprising, administering to a mammal a therapeutically effective amount of a compound of claim 11, or a pharmaceutically acceptable salt, hydrate, solvate or pro-drug thereof and one or more of pharmaceutically acceptable carriers, diluents and excipients.

19. A method of claim 18 in which diseases of the erythroid and hematopoietic systems are selected from the group consisting of: anemia, aplastic anemia, myelodysplastic syndrome, myelosuppression, and cytopenia.

20. A method of treating or preventing diseases selected from the group consisting of: anemia, aplastic anemia, myelodysplastic syndrome, myelosuppression, and cytopenia; comprising, administering to a mammal a therapeutically effective amount of a compound of claim 11, or a pharmaceutically acceptable salt, hydrate, solvate or pro-drug thereof and one or more of pharmaceutically acceptable carriers, diluents and excipients.

21. A pharmaceutical composition including a therapeutically effective amount of a compound of claim 11, or a pharmaceutically acceptable salt, hydrate, solvate or pro-drug thereof and one or more of pharmaceutically acceptable carriers, diluents and excipients.

22. A compound of claim 11 selected from the group consisting of:
(5Z)-2-[(2-Chlorophenyl)amino]-5-[(1-methyl-1H-benzimidazol-6-yl)methylidene]-1,3-thiazol-4(5H)-one;
(5Z)-2-[(2,6-dichlorophenyl)amino]-5-[(1-methyl-1H-benzimidazol-6-yl)methylidene]-1,3-thiazol-4(5H)-one;
(5Z)-2-[(2,6-difluorophenyl)amino]-5-[(1-methyl-1H-benzimidazol-6-yl)methylidene]-1,3-thiazol-4(5H)-one;
(5Z)-2-[(2,4-dimethylphenyl)amino]-5-[(1-methyl-1H-benzimidazol-6-yl)methylidene]-1,3-thiazol-4(5H)-one;
(5Z)-5-[(1-methyl-1H-benzimidazol-6-yl)methylidene]-2-{[2-(methyloxy)phenyl]amino}-1,3-thiazol-4(5H)-one;
(5Z)-5-[(1-methyl-1H-benzimidazol-6-yl)methylidene]-2-{[2-(trifluoromethyl)phenyl]amino}-1,3-thiazol-4(5H)-one;
(5Z)-2-[(2,4-difluorophenyl)amino]-5-[(1-methyl-1H-benzimidazol-6-yl)methylidene]-1,3-thiazol-4(5H)-one;
(5Z)-2-[(2-chloro-4-fluorophenyl)amino]-5-[(1-methyl-1H-benzimidazol-6-yl)methylidene]-1,3-thiazol-4(5H)-one;
(5Z)-2-[(2-Chlorophenyl)-amino]-5-[(1,2-dimethyl-1H-benzimidazol-6-yl)-methylidene]-1,3-thiazol-4(5H)-one;
(5Z)-2-[(2,6-dichlorophenyl)amino]-5-[(1,2-dimethyl-1H-benzimidazol-6-yl)methylidene]-1,3-thiazol-4(5H)-one;
(5Z)-2-[(2,6-difluorophenyl)amino]-5-[(1,2-dimethyl-1H-benzimidazol-6-yl)methylidene]-1,3-thiazol-4(5H)-one;
(5Z)-5-[1,2-dimethyl-1H-benzimidazol-6-yl)methylidene]-2-[(2,4-dimethylphenyl)amino]-1,3-thiazol-4(5H)-one;
(5Z)-2-[(2,4-difluorophenyl)amino]-5-[(1,2-dimethyl-1H-benzimidazol-6-yl)methylidene]-1,3-thiazol-4(5H)-one;
(5Z)-2-[(2-chloro-4-fluorophenyl)amino)-5-[(1,2-dimethyl-1H-benzimidazol-6-yl)methylidene]-1,3-thiazol-4(5H)-one;
(5Z)-2-[(2-Chlorophenyl)-amino]-5-({1-(2-(4-morpholinyl)ethyl]-1H-benzimidazol-6-yl}-methylidene)-1,3-thiazol-4(5H)-one;
(5Z)-2-[(2,6-dichlorophenyl)amino)-5-({1-[2-(4-morpholinyl)ethyl]-1H-benzimidazol-6-yl}methylidene)-1,3-thiazol-4(5H)-one;
(5Z)-2-[(2-chloro-4-fluorophenyl)amino]-5-({1-[2-(4-morpholinyl)ethyl]-1H-benzimidazol-6-yl}methylidene)-1,3-thiazol-4(5H)-one;
(5Z)-2-[(2-chlorophenyl)amino]-5-({1-[2-(dimethylamino)ethyl]-1H-benzimidazol-6-yl}methylidene)-1,3-thiazol-4(5H)-one;
(5Z)-2-[(2,6-dichlorophenyl)amino]-5-({1-[2-(dimethylamino)ethyl]-1H-benzimidazol-6-yl}methylidene)-1,3-thiazol-4(5H)-one;
(5Z)-2-[(2,4-difluorophenyl)amino]-5-({1-[2-(dimethylamino)ethyl]-1H-benzimidazol-6-y}methylidene)-1,3-thiazol-4(5H)-one;
(5Z)-5-({1-[2-(dimethylamino)ethyl]-1H-benzimidazol-6-yl}methylidene)-2-(phenylamino)-1,3-thiazol-4(5H)-one;
(5Z)-2-[(2-chlorophenyl)amino]-5-({1-[2-(diethylamino)ethyl]-1H-benzimidazol-6-yl}methylidene)-1,3-thiazol-4(5H)-one;
(5Z)-2-[(2,6-dichlorophenyl)amino]-5-({1-[2-(diethylamino)ethyl]-1H-benzimidazol-6-yl}methylidene)-1,3-thiazol-4(5H)-one;
(5Z)-2-[2-chlorophenyl)amino]-5-({1-[3(4-morpholinyl)propyl]-1H-benzimidazol-6-yl}methylidene)-1,3-thiazol-4(5H)-one;
(5Z)-2-[(2,6-dichlorophenyl)amino]-5-({1-[3-(4-morpholinyl)propyl]-1H-benzimidazol-6-yl}methylidene)-1,3-thiazol-4(5H)-one;
(5Z)-2-[(2-chlorophenyl)amino]-5-({1-[3-(4-methyl-1-piperazinyl)propyl]-1H-benzimidazol-6-yl}methylidene)-1,3-thiazol-4(5H)-one;
(5Z)-2-[(2,6-dichlorophenyl)amino]-5-({1-(3-(4-methyl-1-piperazinyl)propyl]-1H-benzimidazol-6-yl}methylidene)-1,3-thiazol-4(5H)-one;
(5Z)-2-[(2-chlorophenyl)amino]-5-({1-[2-(1-pyrrolidinyl)ethyl]-1H-benzimidazol-6-yl}methylidene)-1,3-triazol-4(5H)-one;
(5Z)-2-[(2,6-dichlorophenyl)amino]-5-({1-[2-(1-pyrrolidinyl)ethyl]-1H-benzimidazol-6-yl}methylidene)-1,3-thiazol-4(5H)-one;
(5Z)-2-[(2-chlorophenyl)amino]-5-({1-[2-(1-piperidinyl)ethyl]-1H-benzimidazol-6-yl}methylidene)-1,3-thiazol-4(5H)-one;
(5Z)-2-[2,6-dichlorophenyl)amino]-5-({1-[2-(1-piperidinyl)ethyl]-1H-benzimidazol-6-yl}methylidene)-1,3-thiazol-4(5H)-one;
(5Z)-2-[(2,6-difluorophenyl)amino]-5-({1-[2-(1-piperidinyl)ethyl]-1H-benzimidazol-6-yl}methylidene)-1,3-thiazol-4(5H)-one;
(5Z)-2-[(2-Chlorophenyl)amino]-5-({1-[2-(dimethylamino)ethyl]-2-methyl-1H-benzimidazol-6-yl}methylidene-1,3-thiazol-4(5H)-one;
(5Z)-2-[(2,6-dichlorophenyl)amino]-5-({1-[2-(dimethylamino)ethyl]-2-methyl-1H-benzimidazol-6-yl}methylidene)-1,3-thiazol-4(5H)-one;
(5Z)-2-[(2,4-difluorophenyl)amino]-5-({1-[2-(dimethylamino)ethyl]-2-methyl-1H-benzimidazol-6-yl}methylidene)-1,3-thiazol-4(5H)-one;
(5Z)-5-({1-[2-(dimethylamino)ethyl]-2-methyl-1H-benzimidazol-6-yl}methylidene)-2-(phenylamino)-1,3-thiazol-4(5H)-one;
(5Z)-2-[(2,6-dichlorophenyl)amino]-5-({[1-(2-hydroxyethyl)-2-methyl-1H-benzimidazol-6-yl]methylidene}-1,3-thiazol-4(5H)-one;
(5Z)-2-[(2-chlorophenyl)amino]-5-({2-methyl-1-[2-(1-piperidinyl)ethyl]-1H-benzimidazol-6-yl}methylidene)-1,3-thiazol-4(5H)-one;
(5Z)-2-[(2,6-dichlorophenyl)amino]-5-({2-methyl-1-[2-(1-piperidinyl)ethyl]-1H-benzimidazol-6-yl}methylidene)-1,3-thiazol-4(5H)-one;
(5Z)-2-[(2,6-difluorophenyl)amino]-5-({2-methyl-1-[2-(1-piperidinyl)ethyl]-1H-benzimidazol-6-yl}methylidene)-1,3-thiazol-4(5H)-one;
(5Z)-2-[(2-chlorophenyl)amino]-5-[(1-methyl-2-{[2-(4-morpholinyl)ethyl]amino}-1H-benzimidazol-6-yl)methylidene-1,3-thiazol-4(5H)-one;
(5Z)-2-[(2,6-dichlorophenyl)amino]-5-[(1-methyl-2-{[2-(4-morpholinyl)ethyl]amino}-1H-benzimidazol-6-yl)methylidene]-1,3-thiazol-4(5H)-one;
(5Z)-2-[(2,4-difluorophenyl)amino]-5-[(1-methyl-2-{[2-(4-morpholinyl)ethyl]amino}-1H-benzimidazol-6-yl)methylidene]-1,3-thiazol-4(5H)-one;
(5Z)-2-[(2-chlorophenyl)amino]-5-[(2-{[2-(dimethylamino)ethyl]amino}-1-methyl-1H-benzimidazol-6-yl)methylidene]-1,3-thiazol-4(5H)-one;
(5Z)-2-[(2-chlorophenyl)amino]-5-({2-[(2-hydroxyethyl)amino]-1-methyl-1H-benzimidazol-6-yl}methylidene)-1,3-thiazol-4(5H)-one;
(5Z)-2-[(2-chlorophenyl)amino]-5-{[1-methyl-2-(4-morpholinylmethyl)-1H-benzimidazol-6-yl]methylidene}-1,3-thiazol-4(5H)-one;
(5Z)-2-[(2,6-dichlorophenyl)amino]-5-{[1-methyl-2-(4-morpholinylmethyl)-1H-benzimidazol-6-yl]methylidene}-1,3-thiazol-4(5H)-one;
(5Z)-2-[(2-chlorophenyl)amino]-5-({1-methyl-2-[(4-methyl-1-piperazinyl)methyl]-1H-benzimidazol-6-yl}methylidene)-1,3-thiazol-4(5H)-one;
(5Z)-2-[(2,6-dichlorophenyl)amino]-5-({1-methyl-2-[(4-methyl-1-piperazinyl)methyl]-1H-benzimidazol-6-yl}methylidene)-1,3-thiazol-4(5H)-one;
(5Z)-2-[(2-Chlorophenyl)-amino]-5-{[1-methyl-2-(trifluoromethyl)-1H-benzimidazol-6-yl]-methylidene}-1,3-thiazol-4(5H)-one;
(5Z)-2-[(2,6-dichlorophenyl)amino]-5-{[1-methyl-2-(trifluoromethyl)-1H-benzimidazol-6-yl]methylidene}-1,3-thiazol-4(5H)-one;
(5Z)-2-[(2,6-dichlorophenyl)amino]-5-{[1-[2-(dimethylamino)ethyl]-2-(trifluoromethyl)-1H-benzimidazol-6-yl]methylidene}-1,3-thiazol-4(5H)-one;
(5Z)-2-[(2-chlorophenyl)amino]-5-{[2-(1,1-dimethylethyl)-1-methyl-1H-benzimidazol-6-yl]methylidene}-1,3-thiazol-4(5H)-one;
(5Z)-2-[(2,6-dichlorophenyl)amino]-5-{[2-(1,1-dimethylethyl-1-methyl-1H-benzimidazol-6-yl]methylidene}-1,3-thiazol-4(5H)-one;
(5Z)-2-[(2-Chlorophenyl)amino]-5-[(1-methyl-1H-1,2,3-benzotriazol-6-yl)methylidene]-1,3-thiazol-4(5H)-one;
(5Z)-2-[(2,6-dichlorophenyl)amino]-5-[(1-methyl-1H-1,2,3-benzotriazol-6-yl)methylidene]-1,3-thiazol-4(5H)-one;
(5Z)-2-[(2-chlorophenyl)amino]-5-({1-[2-(dimethylamino)ethyl]-1H-1,2,3-benzotriazol-6-yl}methylidene)-1,3-thiazol-4(5H)-one;
(5Z)-2-[(2,6-dichlorophenyl)amino]-5-({1-[2-(dimethylamino)ethyl]-1H-1,2,3-benzotriazol-6-yl}methylidene)-1,3-thiazol-4(5H)-one;
2-(2,6-Dichloro-phenylimino)-5-(2-methyl-benzooxazol-6-yl-methylene)-thiazolidin-4-one;
2-(2,6-Difluoro-phenylimino)-5-(2-methyl-benzooxazol-6-yl-methylene)-thiazolidin-4-one;
2-(2-Fluoro-phenylimino)-5-(2-methyl-benzooxazol-6-yl-methylene)-thiazolidin-4-one;
2-(2-Chloro-phenylamino)-5-(2-methyl-benzooxazol-6-ylmethylene)-thiazolidin-4-one;
2-(2-Trifluromethyl-phenylimino)-5-(2-methyl-benzooxazol-6-ylmethylene)-thiazolidin-4-one;
2-(2,4-Difluoro-phenylimino)-5-(2-methyl-benzooxazol-6-ylmethylene)-thiazolidin-4-one;
2-(2,5-Dichloro-phenylimino)-5-(2-methyl-benzooxazol-6-ylmethylene)-thiazolidin-4-one;
2-(2,4-Dimethyl-phenylimino)-5-(2-methyl-benzooxazol-6-ylmethylene)-thiazolidin-4-one;
2-(4-Cyano-phenylimino)-5-(2-methyl-benzooxazol-6-ylmethylene)-thiazolidin-4-one;
4-[5-(2-Methyl-benzooxazol-6-ylmethylene)-4-oxo-thiazolidin-2-ylideneamino]-benzoic acid;
2-(2,4-Dichloro-phenylimino)-5-(2-methyl-benzooxazol-6-ylmethylene)-thiazolidin-4-one;
2-(2,5-Difluoro-phenylimino)-5-(2-methyl-benzooxazol -6-ylmethylene)-thiazolidin-4-one;
5-(2-Methyl-benzooxazol-6-ylmethylene)-2-phenylimino-thiazolidin-4-one;
5-(2-Methyl-benzooxazol-6-ylmethylene)-2-(2-piperidin-1-yl-ethylimino)-thiazolidin-4-one;
2-(2-Methoxy-ethylimino)-5-(2-methyl-benzooxazol-6-ylmethylene)-thiazolidin-4-one;
5-(2-Methyl-benzooxazol-6-ylmethylene)-2-(3-morpholin-4-yl-propylimino)-thiazolidin-4-one;
3-[5-(2-Methyl-benzooxazol-6-ylmethylene)-4-oxo-thiazolidin-2-ylideneamino]-benzenesulfonamide;
2-(4-Hydroxy-butylimino)-5-(2-methyl-benzooxazol-6-ylmethylene)-thiazolidin-4-one;
2-(trans-4-Hydroxy-cyclohexylimino)-5-(2-methyl-benzooxazol-6-ylmethylene)-thiazolidin-4-one;
5-(2-Methyl-benzooxazol-6-ylmethylene)-2-phenethylimino-thiazolidin-4-one;
4-{2-[5-(2-Methyl-benzooxazol-6-ylmethylene)-4-oxo-thiazolidin-2-ylideneamino]-ethyl}-benzenesulfonamide;
2-(2-Benzo[1,3]dioxol-5-yl-ethylimino)-5-(2-methyl-benzooxazol-6-ylmethylene)-thiazolidin-4-one;
2-(4-Chloro-phenylimino)-5-(2-methyl-benzooxazol-6-ylmethylene)-thiazolidin-4-one;
5-(2-Methyl-benzooxazol-6-ylmethylene)-2-(pyridin-3-ylimino)-thiazolidin-4-one;
3-[5-(2-Methyl-benzooxazol-6-ylmethylene)-4-oxo-thiazolidin-2-ylideneamino]-benzamide;
2-(2-Hydroxy-ethylimino)-5-(2-methyl-benzooxazol-6-ylmethylene)-thiazolidin-4-one;
2-(1-Hydroxymethyl-2-phenyl-ethylimino)-5-(2-methyl-benzooxazol-6-ylmethylene)-thiazolidin-4-one;
N-{6-(2-(2-Bromo-phenylimino)-4-oxo-thiazolidin-5-ylidenemethyl]-1H-benzoimidazol-2-yl}-2-dimethylamino-acetamide;
Methyl (5-{(Z)-[2-[(2-bromophenyl)amino]-4-oxo-1,3-thiazol-5(4H)-ylidene]methyl)-1H-benzimidazol-2-yl)carbamate;
(5Z)-2-[(2-chlorophenyl)amino]-5-{[1-(3,3-dimethylbutyl)-1H-benzimidazol-6-yl]methylidene}-1,3-thiazol-4(5H)-one;
(5Z)-2-[(2,6-dichlorophenyl)amino]-5-{[1-(3,3-dimethylbutyl)-1H-benzimidazol-6-yl]methylidene}-1,3-thiazol-4(5H)-one, trifluoroacetate salt;
(5Z)-5-{[1-(2-cyclopropylethyl)-1H-benzimidazol-6-yl]methylidene)-2-[(2,6-difluorophenyl)amino]-1,3-thiazol-4(5H)-one;
(5Z)-5-{[1-(2-cyclohexylethyl)-1H-benzimidazol-6-yl]methylidene)-2-[(2,6-dichlorophenyl)amino]-1,3-thiazol-4(5H)-one, trifluoroacetate salt;
(2Z,5Z)-2-[(2,6-dichlorophenyl)imino]-5-[(2-phenyl-1H-benzimidazol-6-yl)methylidene]-1,3-thiazolidin-4-one, piperidine salt;
(5Z)-2-[(2-chlorophenyl)amino]-5-{[1-(2-cyclopropylethyl)-1H-benzimidazol-6-yl]methylidene)-1,3-thiazol-4(5H)-one;
(5Z)-5-{[1-(2-cyclopropylethyl)-1-benzimidazol-6-yl]methylidene)-2-[(2,6-dichlorophenyl)amino]-1,3-thiazol-4(5H)-one;
(2Z,5Z)-2-[(2,6-dichlorophenyl)imino]-5-{[2-(1-methylethyl)-1H-benzimidazol-6-yl]methylidene}-1,3-thiazolidin-4-one;
(2Z,5Z)-2-[(2,6-dichlorophenyl)imino]-5-{[2-(2-methylpropyl)-1H-benzimidazol-6-yl]methylidene}-1,3-thiazolidin-4-one, piperidine salt;
(2Z,5Z)-2-[(2,6-dichlorophenyl)imino]-5-{[2-(3-pyridinyl)-1H-benzimidazol-6-yl]methylidene}-1,3-thiazolidin-4 one;
(2Z,5Z)-2-[(2,6-dichlorophenyl)imino]-5-{[2-(hydroxymethyl)-1H-benzimidazol-5-yl]methylidene}-1,3-thiazolidin-4-one;
(2Z,5Z)-2-[(2,6-dichlorophenyl)imino]-5-{[2-(2-hydroxyethyl)-1H-benzimidazol-5-yl]methylidene}-1,3-thiazolidin-4-one;
(2Z,5Z)-2-[(2,6-dichlorophenyl)imino]-5-{[2-(2-pyridinyl)-1H-benzimidazol-6-yl]methylidene}-1,3-thiazolidin-4-one;
5Z)-5-{[1-(2-cyclopentylethyl)-1H-benzimidazol-6-yl]methylidene}-2-[(2,6-dichlorophenyl)amino]-1,3-thiazol-4(5H)-one;
(5Z)-2-[(2-chlorophenyl)amino]-5-{[1-(2-cyclopentylethyl)-1H-benzimidazol-6-yl]methylidene}-1,3-thiazol-4(5H)-one;
(2Z,5Z)-2-[(2,6-dichlorophenyl)amino]-5-[(2-methyl-1H-benzimidazol-6-yl)methylidene]-1,3-thiazolidin-4-one;
(2Z,5Z)-2-[(2,6-dichlorophenyl)imino]-5-{[2-(4-pyridinyl)-1H-benzimidazol-5-yl]methylidene}-1,3-thiazolidin-4-one;
(2Z,5Z)-5-{[1-(2-cyclopropylethyl)-2-(3-pyridinyl)-1H-benzimidazol-6-yl]methylidene)-2-[(2,6-dichlorophenyl)imino]-1,3-thiazolidin-4-one;
(2Z,5Z)-2-[(2,6-dichlorophenyl)imino]-5-{[1-methyl-2-(3-pyridinyl)-1H-benzimidazol-5-yl]methylidene)-1,3-thiazolidin-4-one;
(2Z,5Z)-5-{[2-(aminomethyl)-1H-benzimidazol-5-yl]methylidene}-2-[(2,6-dichlorophenyl)imino]-1,3-thiazolidin-4-one;
(2Z,5Z)-5-(1H-benzimidazol-5-ylmethylidene)-2-[(2,6-dichlorophenyl)imino-1,3-thiazolidin-4-one;
(2Z,5Z)-2-[(2,6-dichlorophenyl)imino]-5-{[2-(hydroxymethyl)-1-methyl-1H-benzimidazol-6-yl]methylidene}-1,3-thiazolidin-4-one;
(5Z)-2-[(2,6dichlorophenyl)amino]-5-({1-(2-(3-pyridinyl)ethyl]-1H-benzimidazol-6-yl}methylidene)-1,3-thiazol-4(5R)-one;
(5Z)-5-{[1-(cyclopropylmethyl)-1H-benzimidazol-6-yl]methylidene)-2-[(2,6-dichlorophenyl)amino]-1,3-thiazol-4(5H)-one,
(5Z)-5-[(1-cyclopentyl-1H-benzimidazol-6-yl)methylidene]-2-[(2,6-dichlorophenyl)amino]-1,3-thiazol-4(5H)-one; and
(5Z)-5-(1,3-Benzoxazol-6-ylmethylidene)-2-[(2,6-dichlorophenyl)amino]-1,3-thiazol-4(5H)-one;
and/or a pharmaceutically acceptable salt, hydrate, solvate or pro-drug thereof.
